(19) United States
(12) Patent Application Publication
(10) Pub. No.: US 2022/0245355 A1
(43) Pub. Date: Aug. 4, 2022

(54) SYSTEM AND METHOD FOR USING A BLOCKCHAIN TO MANAGE KNOWLEDGE IN A HEALTHCARE ECOSYSTEM

(71) Applicant: HEALTHPOINTE SOLUTIONS, INC., Austin, TX (US)

(72) Inventors: Nathan GNANASAMBANDAM, Irvine, CA (US); Mark Henry ANDERSON, Newport Coast, CA (US)

(73) Assignee: HEALTHPOINTE SOLUTIONS, INC., Austin, TX (US)

(21) Appl. No.: 17/611,524

(22) PCT Filed: May 19, 2020
(86) PCT No.: PCT/US2020/033634

§ 371 Date: Nov. 15, 2021

Related U.S. Application Data
(63) Continuation of application No. 16/593,491, filed on Oct. 4, 2019, now Pat. No. 11,263,405.
(60) Provisional application No. 62/743,985, filed on Oct. 10, 2018.
(60) Provisional application No. 62/801,777, filed on Feb. 6, 2019.
(60) Provisional application No. 62/852,051, filed on May 23, 2019.

(51) Int. Cl. G06F 40/30; G16H 10/20; G06F 40/295; G16H 50/20; G06N 5/04; G16H 70/00

(52) U.S. Cl. CPC G06F 40/30; G16H 10/20; G16H 70/00; G16H 50/20; G06N 5/04; G06F 40/295

(57) ABSTRACT

A method for maintaining content pertaining to healthcare in a hyperledger, the method including receiving, from a computing device associated with a medical personnel entity, a transaction request to perform an operation on the hyperledger, wherein the operation includes storing content pertaining to healthcare in the hyperledger. The method also including executing one or more rules of the hyperledger to determine whether to allow the operation to be performed, wherein at least one of the one or more rules includes determining whether the medical personnel entity is associated with an authorizing credential pertaining to healthcare. Responsive to determining that the one or more rules of the hyperledger are satisfied, the method also including performing the operation on the hyperledger to store the content in the hyperledger.

### CROSS-REFERENCE TO RELATED APPLICATION

This application claims priority to and the benefit of U.S. Provisional Pat. App. No. 62/852,051, filed May 23, 2019, the contents of which are incorporated herein by reference in its entirety.

### BACKGROUND

Population health management entails aggregating patient data across multiple health information technology resources, analyzing the data with reference to a single patient, and generating actionable items through which care providers can improve both clinical and financial outcomes. In some instances, coordinating health services to perform the actionable items among multiple entities in a healthcare ecosystem can be a daunting, inefficient, and/or cumbersome task. Further, various health providers may use different treatment plans for treating illnesses or health issues of their patients. The treatment plan used by a first physician may not be as effective as the treatment plan used by another physician. However, the first physician may not be aware of the more effective treatment plan. Further, it may be difficult to verify the source of certain content, such as evidence-based guidelines, clinical processes, clinical trials, treatment plans, etc., in a verifiable manner.

### SUMMARY

A system and method for using a blockchain to manage knowledge in a healthcare ecosystem are disclosed herein. The knowledge may pertain to evidence-based guidelines, knowledge representation, clinical studies, clinical processes, clinical techniques, treatment plans, articles, and so forth. For example, the content may include a treatment plan for treating a medical condition of patients. A physician may develop a unique treatment plan that includes various steps, such as taking medication, drinking a certain amount of fluids, a diet plan, a resting plan, a self-care plan, a monitoring plan, an exercise plan, and so forth for a particular illness, disease, or medical condition. In some embodiments, if the physician has a valid authorizing credential (e.g., medical degree, medical license, national provider identifier), the physician may store the unique treatment plan in the blockchain in a secure manner that further enables the physician to monetize the treatment plan by allowing others to purchase a right to access the treatment plan. In some embodiments, the treatment plans may be modified by other physicians having valid authorizing credentials (e.g., national provider identifiers, medical licenses, etc.). The treatment plans may be verified by other physicians. Further, the disclosed techniques may enable users to validate the source of the content (e.g., evidence-based guidelines, knowledge representation, clinical studies, clinical processes, clinical techniques, treatment plans, articles, etc.) using the blockchain, such that the users may reliably trust the content and use the content.

Although the techniques are disclosed in the context of a healthcare ecosystem, it should be noted that the techniques may be applied to any suitable industry where knowledge management and verifying the source of content is desired and beneficial. For example, certain contracts, patent applications, outlines, books, notes, etc. may be stored and managed using the blockchain in the disclosed embodiments. The knowledge management system using the blockchain may be used to provide a population health management service. The steps of any of the following methods may be implemented as computer instructions stored on tangible, non-transitory media that are executable by one or more processors. Further, the methods may be implemented by a computing device and/or a system including one or more processors.

In one embodiment, a method for maintaining content pertaining to healthcare in a hyperledger includes receiving, from a computing device associated with a medical personnel entity, a transaction request to perform an operation on the hyperledger. The operation includes storing content pertaining to healthcare in the hyperledger. The method also includes executing one or more rules of the hyperledger to determine whether to allow the operation to be performed. At least one of the one or more rules includes determining whether the medical personnel entity is associated with an authorizing credential pertaining to healthcare. Responsive to determining that the one or more rules of the hyperledger are satisfied, the method also includes performing the operation on the hyperledger to store the content in the hyperledger.

In one embodiment, a method for maintaining content pertaining to healthcare in a hyperledger includes receiving, from a computing device associated with a medical personnel entity, a transaction request to perform an operation on the hyperledger. The transaction request includes search criteria, and the operation includes providing, based on the search criteria, content pertaining to healthcare that is stored in the hyperledger. The method also includes executing one or more rules of the hyperledger to determine whether to allow the operation to be performed. At least one of the one or more rules includes determining whether the medical personnel entity has a right to access the content. Responsive to determining that the one or more rules of the hyperledger are satisfied, the method also includes performing the operation on the hyperledger to provide, based on the search criteria, the content pertaining to healthcare to the computing device.

In one embodiment, a method for maintaining documents pertaining to healthcare in a hyperledger includes receiving, from a computing device associated with a medical personnel entity, a transaction request to perform an operation on the hyperledger. The operation includes storing updated content pertaining to healthcare in the hyperledger, and the updated content adds additional content to original content stored in the hyperledger. The method also includes executing one or more rules of the hyperledger to determine whether to allow the operation to be performed. At least one of the one or more rules includes determining whether the additional content in the updated content is new relative to other content pertaining to healthcare stored in the hyperledger. Responsive to determining that the one or more rules of the hyperledger are satisfied, the method also includes performing the operation on the hyperledger to store, in the hyperledger, the updated content including the additional content and the original content.

In one embodiment, a method may include recommending items in conversational streams by receiving conversation stream segments, defining a user action outcome objective based on the conversation stream segments and a user profile that may be stored on a hyperledger, selecting an action likely to advance the user action outcome objective, and presenting a conversation stream segment to motivate an action likely to advance the user action outcome objective.

In one embodiment, a computer-implemented method for providing therapeutic medical action recommendations in response to a medical information natural language conversation stream is disclosed. The method includes receiving segments of a medical information natural language conversation stream at an artificial intelligence-based health information conversation agent from a medical information conversation user interface. Based on the medical information content of a user medical information profile (e.g., stored in a hyperledger) associated with the medical information natural language conversation stream, the method further defines a desired clinical management outcome objective relevant to health management criteria and related health management data attributes of the user medical information profile. The method further involves identifying a set of potential therapeutic interventions correlated to advancement of the clinical management outcome objective. The method further involves selecting from among the set of potential therapeutic interventions correlated to advancement of the clinical management outcome objective a medical intervention likely to advance the clinical management outcome objective. The method further involves presenting in the medical information natural language conversation stream a therapeutic advice conversation stream segment designed to stimulate execution of the medical intervention likely to advance the clinical management outcome objective. The method further involves presenting to the user in the medical information natural language conversation stream a therapeutic advice conversation stream segment explaining a correlation between the medical intervention likely to advance the clinical management outcome objective and achievement of the clinical management outcome objective.

In one embodiment, a computer program product in a non-transitory computer-readable medium for providing therapeutic medical action recommendations in response to a medical information natural language conversation stream is disclosed. The product contains instructions that cause a computer to receive segments of a medical information natural language conversation stream at an artificial intelligence-based health information conversation agent from a medical information conversation user interface. The product contains further instructions that cause the computer to define a clinical management outcome objective relevant to health management criteria and related health management data attributes of the profile in response to the medical information content of a user medical information profile (e.g., stored in a hyperledger) associated with the medical information natural language conversation stream. The product contains further instructions that cause the computer to select a medical intervention likely to advance the clinical management outcome objective. The product contains further instructions that cause the computer to present to the user in the medical information natural language conversation stream a therapeutic advice conversation stream segment designed to stimulate execution of the action likely to advance the clinical management outcome objective.

In one embodiment, a system for providing therapeutic medical action recommendations in response to a medical information natural language conversation stream is disclosed, the system includes a knowledge cloud configured for receiving segments of a medical information natural language conversation stream at an artificial intelligence-based health information from a medical information conversation user interface of a cognitive agent. The system further includes a critical thinking engine. The critical thinking engine is configured to define a clinical management outcome objective relevant to health management criteria and related health management data attributes of the profile in response to medical information content of a user medical information profile (e.g., stored in a hyperledger) associated with the medical information natural language conversation stream in the knowledge cloud. The critical thinking engine is further configured to select a medical intervention likely to advance the clinical management outcome objective. The cognitive agent is configure for presenting to the user in the medical information natural language conversation stream a therapeutic advice conversation stream segment designed to stimulate execution of the action likely to advance the clinical management outcome objective.

In one embodiment, a computer-implemented method for providing action recommendations in response to a user-generated natural language conversation stream is disclosed. The method includes receiving segments of a user-generated natural language conversation stream at an artificial intelligence-based conversation agent from a user interface. The method further includes defining a user action outcome objective relevant to attributes of the profile in response to content of a user profile (e.g., stored in a hyperledger) associated with the user-generated natural language conversation stream. The method further includes selecting an action likely to advance the user action outcome objective. The method further includes presenting to the user in the user-generated natural language conversation stream a conversation stream segment designed to motivate performance of the action likely to advance the user action outcome objective.

### BRIEF DESCRIPTION OF THE DRAWINGS

For a detailed description of example embodiments, reference will now be made to the accompanying drawings in which:

FIG. 1 illustrates, in block diagram form, a system architecture 100 that can be configured to provide a population health management service, in accordance with various embodiments.

FIG. 2 shows additional details of a knowledge cloud, in accordance with various embodiments.

FIG. 3 shows an example subject matter ontology, in accordance with various embodiments.

FIG. 4 shows aspects of a conversation, in accordance with various embodiments.

FIG. 5 shows a cognitive map or “knowledge graph”, in accordance with various embodiments.

FIG. 6 shows a method, in accordance with various embodiments.

FIGS. 7A, 7B, and 7C show methods, in accordance with various embodiments.

FIGS. 8A, 8B, 8C, and 8D show aspects of a user interface, in accordance with various embodiments.

FIGS. 9A and 9B shows aspects of a conversational stream, in accordance with various embodiments.

FIG. 10 shows aspects of a conversational stream, in accordance with various embodiments.

FIG. 11 shows aspects of an action calendar, in accordance with various embodiments.

FIG. 12 shows aspects of a feed, in accordance with various embodiments.

FIG. 13 shows aspects of a hyper-local community, in accordance with various embodiments.

FIG. 14 illustrates a detailed view of a computing device that can represent the computing devices of FIG. 1 used to implement the various platforms and techniques described herein, according to some embodiments.

FIG. 15 shows a method, in accordance with various embodiments.

FIG. 16 shows a method, in accordance with various embodiments.

FIG. 17 shows a method, in accordance with various embodiments.

FIG. 18 shows a therapeutic paradigm logical framework, in accordance with various embodiments

FIG. 19 shows a method, in accordance with various embodiments.

FIG. 20 shows a paradigm logical framework, in accordance with various embodiments.

FIG. 21 shows a method, in accordance with various embodiments.

FIG. 22 shows a method, in accordance with various embodiments.

FIG. 23 shows a distributed network of nodes each maintaining a copy of a hyperledger to manage content, in accordance with various embodiments.

FIG. 24 shows an example hyperledger, in accordance with various embodiments.

FIG. 25A shows a cognitive map or “knowledge graph” at a first knowledge stage, in accordance with various embodiments.

FIG. 25B shows the cognitive map or “knowledge graph” of FIG. 25A evolved to a second knowledge stage, in accordance with various embodiments.

FIG. 26 shows a general process for performing transaction requests on a hyperledger by various nodes in a healthcare ecosystem, in accordance with various embodiments.

FIG. 27 shows an example content stored on the hyperledger, in accordance with various embodiments.

FIG. 28 shows a method, in accordance with various embodiments.

FIG. 29 shows an example search for content, in accordance with various embodiments.

FIG. 30 shows a method, in accordance with various embodiments.

FIG. 31 shows example updated content stored in the hyperledger, in accordance with various embodiments.

FIG. 32 shows a method, in accordance with various embodiments.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

### DETAILED DESCRIPTION

The following discussion is directed to various embodiments of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

There are numerous entities involved in various transactions in a healthcare ecosystem. For example, the entities may include patients (consumers), medical personnel (e.g., physicians, nurses, pharmacists, dentists, optometrists, orthodontists, etc.), insurance providers, clinics, hospitals, pharmacies, professional associations, government agencies, and so forth. Example transactions in the healthcare ecosystem may include a patient requesting content pertaining to healthcare, a physician providing content pertaining to healthcare, a physician verifying content pertaining to healthcare, a physician updating content pertaining to healthcare, a physician deleting content pertaining to healthcare, and so forth. The content may include evidence-based guidelines (e.g., published by one or more physicians, a professional association, and/or government agency), knowledge representations, clinical studies, clinical processes, clinical techniques, treatment plans, and so forth. The content may be presented in one or more documents (e.g., word processing document, spreadsheet document, slideshow document), videos, images, and so forth. In some instances, the content may be a combination of information presented in different types of documents (e.g., a video embedded in a word processing document including text).

Medical personnel entities, such as physicians, may generate content (e.g., treatment plans) for particular medical conditions (e.g., illnesses, diseases, etc.). The treatment plans may include steps for a patient to take to recover from the medical condition and/or to reduce symptoms of the medical condition. For example, the steps may relate to a type of medication to take and a schedule for taking the medication, an exercise plan, a diet plan, a rest plan, and so forth for a patient. The treatment plans may be individually tailored for characteristics (e.g., age, weight, height, gender, active level, etc.) and/or nuances of each patient.

In some instances, the physicians may not share the treatment plans with one another in an effort to earn business of patients by offering a proprietary treatment plan. Physician A may have his own treatment plan for diabetes that is different than another treatment plan that is used by physician B. The efficacies of the treatment plans may vary. For example, if followed, physician A's treatment plan may provide better results (e.g., cures an illness, faster recovery time, reduces symptoms, etc.) for a patient than physician B's treatment plan. Physician B may desire to use physician A's treatment plan but may not have access to physician A's treatment plan. Currently, there is no reliably secure and efficient technique to share the content between physicians, or a reliably secure and efficient technique for other physicians to review, verify, and/or modify the treatment plans. It may be advantageous to the physicians to profit from their knowledge that is encompassed in their unique treatment plans. Conventional systems do not provide a way for the treatment plans to be monetized in a secure and verifiable way such that physicians may purchase and/or acquire access rights to desired treatment plans.

Accordingly, the disclosed embodiments generally relate to techniques for managing content (e.g., evidence-based guideline, knowledge representations, clinical studies, clinical processes, clinical techniques, treatment plans, etc.) using a blockchain. A blockchain may refer to an immutable ledger for recording transactions. The cognitive intelligence platform integrates and consolidates data/information from various sources and entities and provides a population health management service. In some embodiments, at least some of the data/information from the various sources and entities may be stored in the blockchain. The blockchain may be maintained by a distributed network of nodes. In some embodiments, a consensus protocol may be used by the nodes to determine whether to allow transactions to be performed and groups the transactions into blocks that are added to the blockchain.

There are different kinds of blockchains, such as permissionless and permissioned. In a permissionless blockchain, any entity may participate without an identity. In a permissioned blockchain, each entity that participates in the blockchain is identified and known. An example of a permissioned blockchain is a hyperledger. The permissions cause the participating nodes to view only the appropriate transactions in the hyperledger. Programmable logic may be implemented as rules that are executed by the hyperledger. In some embodiments, the rules may be analytics-based and may specify scenarios when updates to the hyperledger are to be made by the various entities of the healthcare ecosystem. Using the analytics-based rules may make each node an active participant by updating the hyperledger at specified times.

The hyperledger may provide a verifiable trace of proof that the content stored on the hyperledger is associated with entities having authorized credentials (e.g., medical license) to facilitate more efficient verification of the information, among other things. The hyperledger may provide a secure chain of record that is used to enhance the efficiency and/or security of the knowledge management process in the healthcare ecosystem. An objective process of administering and managing clinical knowledge can be achieved using the hyperledger in disclosed embodiments. A user's experience using a computer may be improved using the disclosed embodiments by verifying the source of content in a secure manner, such that the user is confident that the content is trustworthy because it was written by a medical personnel entity having valid authorizing credentials, has been recently updated by a medical personnel entity having valid authorizing credentials, and/or was vetted by other medical personnel entities having valid authorizing credentials. Further, network, processor, and/or memory resources may be reduced using the disclosed techniques by the hyperledger returning ranked content that is (i) written by a medical personnel entity having a stellar reputation, (ii) viewed by a threshold number of medical personnel entities, and/or (iii) verified as being valid by a threshold number of medical personnel entities because the user may select content initially presented based on one or more of these factors without performing additional searches.

Each entity in the healthcare ecosystem may register as a node in a distributed, decentralized network. Registering a node for an entity may involve a transaction that is added to the hyperledger. Each node may maintain a respective copy of the hyperledger as a shared single source of truth. During registration, each entity may provide certain information pertaining to the entity to be maintained by the hyperledger at the nodes. For example, a physician may register as a node and may provide information (e.g., National Provider Identifier (NPI), license number, date licensed, date license last updated, etc.) pertaining to their authorizing credential, specialty of medical practice, location of practice, and any other information relevant to practicing in the healthcare ecosystem. A pharmacist may register as a node and may provide information (e.g., license number, date licensed, date license last updated, etc.) pertaining to their authorizing credential, location of practice, and any other information relevant to practicing in the healthcare ecosystem. A patient may register as a node and may provide personal information (e.g., driver's license number, social security number, name, insurance provider number, type of insurance, address, medical records, allergies, etc.) that enables verifying their identity and establishing a user profile, among other things. A non-patient user may also register as a node by providing personal information. A news organization may also register as a node by providing an authorizing credential associated with its entity type.

In some embodiments, just the entities that are registered as nodes may add content to the hyperledger. For example, in the context of a social media forum, using the disclosed techniques may prevent a user without a node to publish misleading and potentially untrue information on the social media forum.

A computer-implemented application may be accessible on a computing device of each entity. The application may be written in computer instructions that are stored on one or more memory devices of the computing device and executable by one or more processing devices of the computing device. In some embodiments, the application may be a stand-alone application that is installed on the computing device, while in other embodiments, the application may be executable via another application (e.g., a website in a web browser).

A medical personnel entity may use the application to store documents on the hyperledger. For example, a medical personnel entity may use the application to submit a transaction request to perform an operation on the hyperledger. The operation may include storing content (e.g., knowledge representation, treatment plan, etc.) on the hyperledger. One or more rules of the hyperledger may be executed prior to allowing the operation to be performed. The rules may be logic implemented in computer instructions and installed in the hyperledger. One of the rules may determine whether the medical personnel entity that submitted the transaction request is associated with a valid authorizing credential (e.g., medical degree, medical license number). Another rule may determine whether the content includes any portions that are new relative to other content stored on the hyperledger. For example, the rule may prevent duplicated knowledge from being added to the hyperledger. That is, at least a portion of the content being added may be required to be new and unique and not disclosed by other content on the hyperledger.

Each entity may use the application to search for desired content, such as treatment plans, on the hyperledger. The content may be associated with various access rights. For example, when stored, the content can be set to public such that anyone using the application can obtain the content. The content may be set to private such that a user has to have a certain access right to obtain the content. In some embodiments, a user may purchase an access right to particular content and the author of that particular content may profit. In some embodiments, users that are part of a same organization (e.g., hospital) may have access rights to content associated with the users of that organization.

The hyperledger enables tracing the content to a source so a user can verify that the content was generated by a medical personnel entity having a valid authorizing credential, for example. Further, the hyperledger may record how many licensed medical personnel entities have viewed a particular content, have verified the particular content, have edited the particular content, a timestamp of the latest update to the particular content, whether the content is still valid, and so forth. A user may view a time series of when the content was created and when the content was updated over time. Further, a date at which the content is required to be updated may also be presented by the application. The hyperledger may enable content to evolve with additional content over time and provides security to ensure that the content is modified by licensed professionals.

The cognitive intelligence platform has the ability to extract concepts, relationships, and draw conclusions from a given text posed in natural language (e.g., a passage, a sentence, a phrase, and a question) by performing conversational analysis which includes analyzing conversational context. For example, the cognitive intelligence platform has the ability to identify the relevance of a posed question to another question.

The benefits provided by the cognitive intelligence platform, in the context of healthcare, include freeing up physicians from focusing on day to day population health management. Thus a physician can focus on her core competency—which includes disease/risk diagnosis and prognosis and patient care. The cognitive intelligence platform provides the functionality of a health coach and includes a physician's directions in accordance with the medical community's recommended care protocols and also builds a systemic knowledge base for health management. The cognitive intelligence platform may leverage the information stored in the hyperledger to recommend certain actions be taken by a patient. For example, using the hyperledger, the recommended actions may include setting up a consultation with a physician having a valid authorizing credential at a location near the patient (e.g., based on geolocations of devices of the entities).

The cognitive intelligence platform may implement an intuitive conversational cognitive agent that engages in a question and answering system that is human-like in tone and response. The described cognitive intelligence platform endeavors to compassionately solve goals, questions and challenges. Further, the cognitive intelligence platform may use a hyperledger to manage knowledge between entities in a healthcare ecosystem more efficiently and/or securely. The described methods and systems are described as occurring in the healthcare space, though other areas are also contemplated.

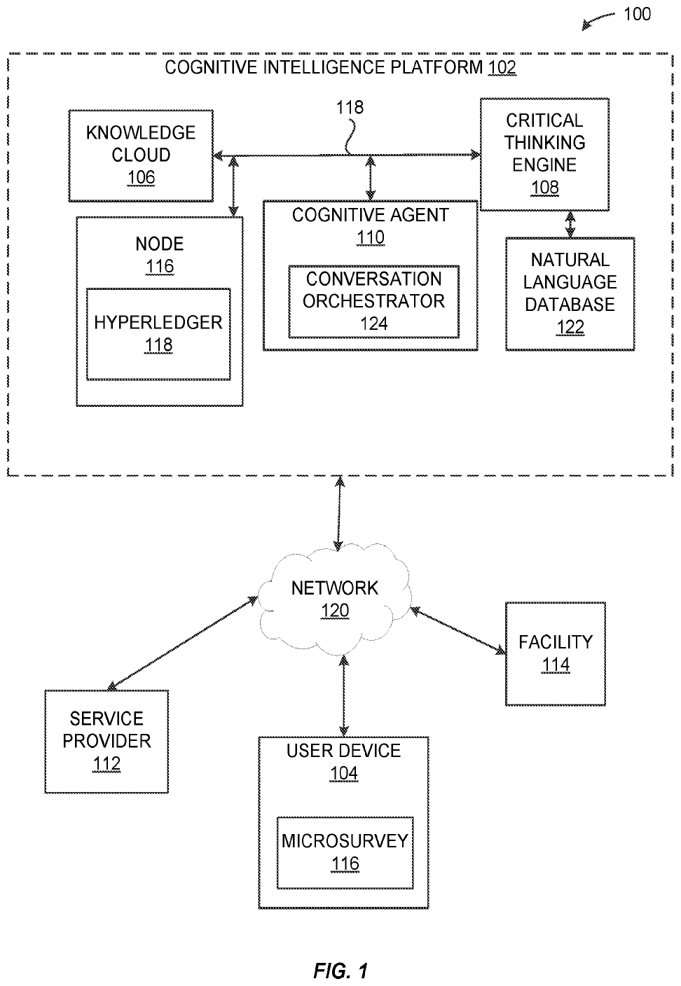

FIG. 1 shows a system architecture 100 that can be configured to provide a population health management service, in accordance with various embodiments. Specifically, FIG. 1 illustrates a high-level overview of an overall architecture that includes a cognitive intelligence platform 102 communicably coupled to a user device 104. The cognitive intelligence platform 102 includes several computing devices, where each computing device, respectively, includes at least one processor, at least one memory, and at least one storage (e.g., a hard drive, a solid-state storage device, a mass storage device, and a remote storage device). The individual computing devices can represent any form of a computing device such as a desktop computing device, a rack-mounted computing device, and a server device. The foregoing example computing devices are not meant to be limiting. On the contrary, individual computing devices implementing the cognitive intelligence platform 102 can represent any form of computing device without departing from the scope of this disclosure.

The several computing devices work in conjunction to implement components of the cognitive intelligence platform 102 including: a knowledge cloud 106; a critical thinking engine 108; a natural language database 122; a cognitive agent 110; and a node 116. The cognitive intelligence platform 102 is not limited to implementing only these components, or in the manner described in FIG. 1. That is, other system architectures can be implemented, with different or additional components, without departing from the scope of this disclosure. The example system architecture 100 illustrates one way to implement the methods and techniques described herein.

The node 116 represents a single computing device in a distributed blockchain network of nodes 116 (also referred to as a distributed hyperledger fabric herein) of the cognitive intelligence platform 102. A permissioned type of blockchain, referred to as a hyperledger 118, may be implemented and a respective copy of the hyperledger 118 may be stored on a respective node 116. The nodes 116 may represent any suitable entity in a healthcare ecosystem. For example, some of the entities may include a service provider 112 (e.g., medical personnel entity, such as a physician, dentist, pharmacist, optometrist, orthodontic, nurse, etc.), a facility 114 (e.g., medical facility entity), a patient entity, and so forth. Each entity may be associated with a respective computing device that they use to register as a node on the blockchain network and request transactions to be performed using the hyperledger 118.

In a permissioned blockchain, such as the hyperledger 118, the entities register by providing certain information to the hyperledger. Based on one or more rules associated with the hyperledger 118, the entity may register as a node 116 on the blockchain network and be provided with authenticating credentials that are used to identify the entities when they request transactions to be performed on the hyperledger 118. The rules may be executable software modules that are installed in the hyperledger 118 itself. In some instances, when a user sends a transaction request to the hyperledger 118, the hyperledger 118 may invoke the rules, which perform functions depending on the type of transaction being requested. In addition, the nodes 116 may employ a consensus protocol whereby the nodes 116 communicate with each other to determine whether to allow the transaction to be performed to modify the hyperledger 118.

The entities use computing devices to send requests to perform transactions using the hyperledger 118 to the cognitive intelligence platform 102. The transactions may include performing various operations. When applicable rules and/or the consensus protocol is satisfied, the operation in the requested transactions may be completed (e.g., storing content on the hyperledger 118, verifying content on the hyperledger 118, editing a treatment plan 118 on the hyperledger 118, transmitting content to a user device 104, etc.) and a record of the transaction may be added to the hyperledger 118. In some instances, the transactions may not be altered or removed, thereby providing an immutable quality to the hyperledger 118. Further, cryptography may be used to secure the hyperledger 118 and the messages between the nodes 116 of the blockchain network and/or the computing devices requesting the transactions. In some embodiments, just the authorized entities are allowed to perform the transactions on the hyperledger 118, and in some instances, just the appropriate entities are allowed to view details of particular transactions in the hyperledger 118.

In some embodiments, a request to register as a node 116 may be a type of transaction that is recorded in the hyperledger 118. The entities may send the requests to register as a node 116 using the hyperledger 118, and the requests include certain information pertaining to the entities. For example, a medical personnel entity may provide an authorizing credential, such as a medical license number. If the rules and/or the consensus protocol is satisfied, the entity may be associated with a node 116. Further, the hyperledger 118 may be updated by adding a block storing the transaction including the information pertaining to the entity that is associated with the node 116. The updated hyperledger 118 may be stored at the node for the entity. In some embodiments, the copies of the other hyperledgers 118 at the other nodes 116 in the blockchain network may be updated with the new transaction. Further, when the entity is registered as a node 116, the computing device associated with that entity may be provided with authenticating credentials for that entity. The computing device may use the authenticating credentials to make subsequent requests to the hyperledger 118. The authenticating credentials may be a username, password, hash code, or the like that uniquely identify an identity of the entity. The entities (e.g., physician, patient, etc.) may use a software application running on a computing device to submit the transaction requests to the hyperledger 118.

The hyperledger 118 may be used as a verifiable trace of proof to determine that the source of certain content (e.g., treatment plans) were generated and provided by licensed entities (e.g., medical doctors) having valid authorizing credentials. The hyperledger 118 may execute its rules when a request to upload content is received, when a request to modify a stored content is received, when a request to verify content is received, when a request to view content is received, and so forth. In some embodiments, the rules may require that the entity be associated with the authenticating credentials, the entity be associated with the authorizing credential, and/or the content that is requested to be added is new prior to allowing the transaction to be performed.

The knowledge cloud 106 represents a set of instructions executing within the cognitive intelligence platform 102 that implement a database configured to receive inputs from several sources and entities. For example, some of the sources and entities include a service provider 112, a facility 114, and a microsurvey 116—each described further below.

The critical thinking engine 108 represents a set of instructions executing within the cognitive intelligence platform 102 that execute tasks using artificial intelligence, such as recognizing and interpreting natural language (e.g., performing conversational analysis), and making decisions in a linear manner (e.g., in a manner similar to how the human left brain processes information). Specifically, an ability of the cognitive intelligence platform 102 to understand natural language is powered by the critical thinking engine 108. In various embodiments, the critical thinking engine 108 includes a natural language database 122. The natural language database 112 includes data curated over at least thirty years by linguists and computer data scientists, including data related to speech patterns, speech equivalents, and algorithms directed to parsing sentence structure.

Furthermore, the critical thinking engine 108 is configured to deduce causal relationships given a particular set of data, where the critical thinking engine 108 is capable of taking the individual data in the particular set, arranging the individual data in a logical order, deducing a causal relationship between each of the data, and drawing a conclusion. The ability to deduce a causal relationship and draw a conclusion (referred to herein as a “causal” analysis) is in direct contrast to other implementations of artificial intelligence that mimic the human left brain processes. For example, the other implementations can take the individual data and analyze the data to deduce properties of the data or statistics associated with the data (referred to herein as an “analytical” analysis). However, these other implementations are unable to perform a causal analysis—that is, deduce a causal relationship and draw a conclusion from the particular set of data. As described further below—the critical thinking engine 108 is capable of performing both types of analysis: causal and analytical.

The cognitive agent 110 represents a set of instructions executing within the cognitive intelligence platform 102 that implement a client-facing component of the cognitive intelligence platform 102. The cognitive agent 110 is an interface between the cognitive intelligence platform 102 and the user device 104. And in some embodiments, the cognitive agent 110 includes a conversation orchestrator 124 that determines pieces of communication that are presented to the user device 104 (and the user). When a user of the user device 104 interacts with the cognitive intelligence platform 102, the user interacts with the cognitive agent 110. The several references herein, to the cognitive agent 110 performing a method, can implicate actions performed by the critical thinking engine 108, which accesses data in the knowledge cloud 106, the natural language database 122, and/or the hyperledger 118.

In various embodiments, the several computing devices executing within the cognitive intelligence platform are communicably coupled by way of a network/bus interface. Furthermore, the various components (e.g., the knowledge cloud 106, the critical thinking engine 108, the cognitive agent 110, and the node 116), are communicably coupled by one or more inter-host communication protocols 118. In one example, the knowledge cloud 106 is implemented using a first computing device, the critical thinking engine 108 is implemented using a second computing device, the cognitive agent 110 is implemented using a third computing device, and the node 116 is a fourth computing device, where each of the computing devices are coupled by way of the inter-host communication protocol 118. Although in this example, the individual components are described as executing on separate computing devices this example is not meant to be limiting, the components can be implemented on the same computing device, or partially on the same computing device, without departing from the scope of this disclosure.

The user device 104 represents any form of a computing device, or network of computing devices, e.g., a personal computing device, a smart phone, a tablet, a wearable computing device, a notebook computer, a media player device, and a desktop computing device. The user device 104 includes a processor, at least one memory, and at least one storage. A user uses the user device 104 to input a given text posed in natural language (e.g., typed on a physical keyboard, spoken into a microphone, typed on a touch screen, or combinations thereof) and interacts with the cognitive intelligence platform 102, by way of the cognitive agent 110.

A user (e.g., patient entity) may also use a software application installed on the user device 104 to request transactions to be performed using authenticating credentials provided to the user device 104 during registration of the user as a node 116 on the blockchain network. Such an implementation makes the blockchain node 116 an active participant in the hyperledger 118. In some embodiments, the transactions may request to access certain content stored on the hyperledger 118. For example, a user may desire to view a treatment plan for diabetes. In some instances, the hyperledger 118 can execute one or more rules to determine which treatment plan for diabetes was written by the most prestigious physician (e.g., based on peer and/or patient reviews), by physicians with medical degrees from certain medical schools, has been verified by the most or a threshold amount of physicians, has been viewed by the most or a threshold amount of physicians, is valid within a certain time period, and the like.

In some embodiments, the user may obtain content from a doctor's office, a public kiosk, a web site, or the like and may desire to verify the source of the content and determine whether it is trustworthy. The user may use the software application to search for that particular content and the hyperledger 118 may provide information to the user device 104 pertaining to the content, such as who the author of the content is, whether the author is associated with a valid authorizing credential (e.g., medical license), whether the content has been verified by other medical personnel entities, how many times other medical personnel entities have viewed and/or used the content, and so forth. Based on the information presented by the software application, the user may determine whether to trust and/or use the content.

The architecture 100 includes a network 120 that communicatively couples various devices, including the cognitive intelligence platform 102 and the user device 104. The network 120 can include local area network (LAN) and wide area networks (WAN). The network 102 can include wired technologies (e.g., Ethernet®) and wireless technologies (e.g., Wi-Fi®, code division multiple access (CDMA), global system for mobile (GSM), universal mobile telephone service (UMTS), Bluetooth®, and ZigBee®. For example, the user device 104 can use a wired connection or a wireless technology (e.g., Wi-Fi®) to transmit and receive data over the network 120.

Still referring to FIG. 1, the knowledge cloud 106 is configured to receive data from various sources and entities and integrate the data in a database. An example source that provides data to the knowledge could 106 is the service provider 112, an entity that provides a type of service to a user. For example, the service provider 112 can be a health service provider (e.g., a doctor's office, a physical therapist's office, a nurse's office, or a clinical social worker's office), and a financial service provider (e.g., an accountant's office). For purposes of this discussion, the cognitive intelligence platform 102 provides services in the health industry (e.g., a healthcare ecosystem), thus the examples discussed herein are associated with the health industry. However, any service industry can benefit from the disclosure herein, and thus the examples associated with the health industry are not meant to be limiting.

Throughout the course of a relationship between the service provider 112 and a user (e.g., the service provider 112 provides healthcare to a patient), the service provider 112 collects and generates data associated with the patient or the user, including health records that include doctor's notes and prescriptions, billing records, and insurance records. The service provider 112, using a computing device (e.g., a desktop computer or a tablet), provides the data associated with the user to the cognitive intelligence platform 102, and more specifically the knowledge cloud 106. This data associated with the user may be stored in the hyperledger 118, in some embodiments.

In some embodiments, the service provider 112 may be logged into a software application on a computing device that communicates with the cognitive intelligence platform 102 via the cognitive agent 110. The service provider 112 may use the software application to make transaction requests to the hyperledger 118 at a node 116 associated with the service provider 112. For example, a medical personnel entity (e.g., service provider 112) may use the software application to request to store content at the hyperledger 118. The content may pertain to healthcare and may include a new portion of knowledge/information that the medical personnel entity discovered or decided to include in a treatment plan. In one example, the content may include a self-care component to a diabetes treatment plan that the medical personnel entity determined provides better outcomes for patients.

When the transmission request is received, the hyperledger 118 may use one or more rules to determine whether the service provider 112 has proper authenticating credentials for a registered node 116 on the blockchain network. The hyperledger 118 may use one or more rules to determine whether the service provider 112 is associated with a valid authorizing credential on the hyperledger 118. The hyperledger 118 may use one or more rules to determine whether at least a portion of the content provided by the service provider 112 is new relative to other content stored on the hyperledger 118. For example, the hyperledger 118 may analyze other pieces of content related to diabetes treatment plans that are stored on the hyperledger 118 to determine if any of them disclose the particular methodology of self-care added to the submitted content by the service provider 112. In some embodiments, the content submitted and the other content stored on the hyperledger 118 may be parsed and compared (e.g., string comparisons) to determine if there is matching text. If a threshold amount of the content matches between the submitted content and the other content stored on the hyperledger 118, the one or more rules may determine that the submitted content is not new. If each of the applicable one or more rules are satisfied and/or a consensus of nodes 116 approve the transaction, then the content may be stored on the hyperledger 118. If any of the one or more rules described above are not satisfied and/or the consensus of nodes 116 do not approve the transaction, then the submitted content may not be stored on the hyperledger 118.

In another example, a medical personnel entity (e.g., service provider 112) may use the software application to request to view and/or verify content stored on the hyperledger 118. When the transmission request is received, the hyperledger 118 may use one or more rules to determine whether the service provider 112 has proper authenticating credentials for a registered node 116 on the blockchain network. The hyperledger 118 may use one or more rules to determine whether the service provider 112 has a proper access right to access the content and/or whether the service provider 112 is associated with a valid authorizing credential on the hyperledger 118. In some embodiments, the content may be set to private and the service provider 112 may purchase a right to access the content. In other instances, where the service provider 112 is a part of a same organization as the author of the content, the service provider 112 may be granted the access right.

A computing device associated with the service provider 112 may be provided with the requested content. The hyperledger 118 may be updated to reflect that the hyperledger 118 has been viewed by the service provider 118. The service provider 112 may review the content and transmit, via a computing device, a transaction request to the hyperledger 118 where the transaction request includes an operation to verify the content. Verifying the content may include the hyperledger 118 using one or more rules to determine that the service provider 112 is associated with a valid authorizing credential. If the one or more rules are satisfied and/or a consensus of nodes 116 approve the transaction, the hyperledger 112 may update a record to indicate that this particular content has been verified by another service provider 112 besides the author/creator of the content.

In some embodiments, once the content is received by the computing device of the requesting service provider 112, the service provider 112 may modify the content, by adding additional content (e.g., a video of additional steps) not disclosed in the original content, and transmit a transaction request to the hyperledger 118 to store the updated content including the additional content (video) and the original content. The hyperledger 118 may use one or more rules to determine whether the service provider 112 is associated with a valid authorizing credential and/or whether the updated content includes new content that is not disclosed by other content in the hyperledger 118. If the one or more rules and/or the consensus protocol are satisfied, the updated content may be stored on the hyperledger 118.

Another example source that provides data to the knowledge cloud 106 is the facility 114 (e.g., medical facility entity). The facility 114 represents a location owned, operated, or associated with any entity including the service provider 112. As used herein, an entity represents an individual or a collective with a distinct and independent existence. An entity can be legally recognized (e.g., a sole proprietorship, a partnership, a corporation) or less formally recognized in a community. For example, the entity can include a company that owns or operates a gym (facility). Additional examples of the facility 114 include, but is not limited to, a hospital, a trauma center, a clinic, a dentist's office, a pharmacy, a store (including brick and mortar stores and online retailers), an out-patient care center, a specialized care center, a birthing center, a gym, a cafeteria, and a psychiatric care center.

As the facility 114 represents a large number of types of locations, for purposes of this discussion and to orient the reader by way of example, the facility 114 represents the doctor's office or a gym. The facility 114 generates additional data associated with the user such as appointment times, an attendance record (e.g., how often the user goes to the gym), a medical record, a billing record, a purchase record, an order history, and an insurance record. The facility 114, using a computing device (e.g., a desktop computer or a tablet), provides the data associated with the user to the cognitive intelligence platform 102, and more specifically the knowledge cloud 106. This data associated with the user may be stored in the hyperledger 118, in some embodiments.

For example, the facility 114 may use a computing device associated with the facility to make requests for transactions to be performed using authenticating credentials provided to the computing device during registration of the facility 114 as a node 116 on the blockchain network. In some embodiments, the transactions may include requesting storage, on the hyperledger 118, of evidence-based guidelines that are generated as a result of clinical trials or studies performed by medical personnel entities of the facility 114, and/or of results of the clinical trials, studies, and so forth on the hyperledger 118. The facility 114 may also transmit, using the computing device, a transaction request to view content stored on the hyperledger 118. For example, when a patient is consulting a physician at the facility 114, the facility may request information pertaining to a treatment plan to provide to the patient. The information may be transmitted to a computing device of the patient (e.g., user device 104) and the user may use the software application to verify the source of the content, when the content was generated, whether the source/creator of the content is associated with a valid authorizing credential, whether the content has been verified within a certain time frame, and so forth.

An additional example source that provides data to the knowledge cloud 106 is the microsurvey 116. The microsurvey 116 represents a tool created by the cognitive intelligence platform 102 that enables the knowledge cloud 106 to collect additional data associated with the user. The microsurvey 116 is originally provided by the cognitive intelligence platform 102 (by way of the cognitive agent 110) and the user provides data responsive to the microsurvey 116 using the user device 104. Additional details of the microsurvey 116 are described below.

Yet another example source that provides data to the knowledge cloud 106, is the cognitive intelligence platform 102, itself. In order to address the care needs and well-being of the user, the cognitive intelligence platform 102 collects, analyzes, and processes information from the user, healthcare providers, and other eco-system participants, and consolidates and integrates the information into knowledge. The knowledge can be shared with the user and stored in the knowledge cloud 106.

In various embodiments, the computing devices used by the service provider 112 and the facility 114 are communicatively coupled to the cognitive intelligence platform 102, by way of the network 120. While data is used individually by various entities including: a hospital, practice group, facility, or provider, the data is less frequently integrated and seamlessly shared between the various entities in the current art. The cognitive intelligence platform 102 provides a solution that integrates data from the various entities. That is, the cognitive intelligence platform 102 ingests, processes, and disseminates data and knowledge in an accessible fashion, where the reason for a particular answer or dissemination of data is accessible by a user.

In particular, the cognitive intelligence platform 102 (e.g., by way of the cognitive agent 110 interacting with the user) holistically manages and executes a health plan for durational care and wellness of the user (e.g., a patient or consumer). The health plan includes various aspects of durational management that is coordinated through a care continuum.

The cognitive agent 110 can implement various personas that are customizable. For example, the personas can include knowledgeable (sage), advocate (coach), and witty friend (jester). And in various embodiments, the cognitive agent 110 persists with a user across various interactions (e.g., conversations streams), instead of being transactional or transient. Thus, the cognitive agent 110 engages in dynamic conversations with the user, where the cognitive intelligence platform 102 continuously deciphers topics that a user wants to talk about. The cognitive intelligence platform 102 has relevant conversations with the user by ascertaining topics of interest from a given text posed in a natural language input by the user. Additionally the cognitive agent 110 connects the user to healthcare service providers, hyperlocal health communities, and a variety of services and tools/devices, based on an assessed interest of the user. In some embodiments, the cognitive agent 110 may connect the user to healthcare service providers that are in a vicinity of the geolocation of the user device 104 based on certain information stored in the hyperledger 118 (e.g., which healthcare service providers have valid authorizing credentials, location of the healthcare service providers, specialty of the healthcare service provider, health issue of the patient, etc.).

As the cognitive agent 110 persists with the user, the cognitive agent 110 can also act as a coach and advocate while delivering pieces of information to the user based on tonal knowledge, human-like empathies, and motivational dialog within a respective conversational stream, where the conversational stream is a technical discussion focused on a specific topic. Overall, in response to a question—e.g., posed by the user in natural language—the cognitive intelligence platform 102 consumes data from and related to the user and computes an answer. The answer is generated using a rationale that makes use of common sense knowledge, domain knowledge, evidence-based medicine guidelines, clinical ontologies, and curated medical advice. Thus, the content displayed by the cognitive intelligence platform 102 (by way of the cognitive agent 110) is customized based on the language used to communicate with the user, as well as factors such as a tone, goal, and depth of topic to be discussed.

Overall, the cognitive intelligence platform 102 may be accessible to a user (e.g., patient entity), medical facility entities (e.g., a hospital system, clinics, pharmacies), medical personnel entities (e.g., physicians, pharmacists, dentists, optometrists, etc.), insurance provider entities, professional association entities, and government agency entities. Additionally, the cognitive intelligence platform 102 is accessible to paying entities interested in user behavior—e.g., the outcome of physician-consumer interactions in the context of disease or the progress of risk management. Additionally, entities that provides specialized services such as tests, therapies, and clinical processes that need risk based interactions can also receive filtered leads from the cognitive intelligence platform 102 for potential clients.

Conversational Analysis

In various embodiments, the cognitive intelligence platform 102 is configured to perform conversational analysis in a general setting. The topics covered in the general setting is driven by the combination of agents (e.g., cognitive agent 110) selected by a user. In some embodiments, the cognitive intelligence platform 102 uses conversational analysis to identify the intent of the user (e.g., find data, ask a question, search for facts, find references, and find products) and a respective micro-theory in which the intent is logical.

For example, the cognitive intelligence platform 102 applies conversational analysis to decode what the user is asking or stated, where the question or statement is in free form language (e.g., natural language). Prior to determining and sharing knowledge (e.g., with the user or the knowledge cloud 106), using conversational analysis, the cognitive intelligence platform 102 identifies an intent of the user and overall conversational focus.

The cognitive intelligence platform 102 responds to a statement or question according to the conversational focus and steers away from another detected conversational focus so as to focus on a goal defined by the cognitive agent 110. Given an example statement of a user, “I want to fly out tomorrow,” the cognitive intelligence platform 102 uses conversational analysis to determine an intent of the statement. Is the user aspiring to be bird-like or does he want to travel? In the former case, the micro-theory is that of human emotions whereas in the latter case, the micro-theory is the world of travel. Answers are provided to the statement depending on the micro-theory in which the intent logically falls.

The cognitive intelligence platform 102 utilize a combination of linguistics, artificial intelligence, and decision trees to decode what a user is asking or stating. The discussion includes methods and system design considerations and results from an existing embodiment. Additional details related to conversational analysis are discussed next.

Analyzing Conversational Context as Part of Conversational Analysis

For purposes of this discussion, the concept of analyzing conversational context as part of conversational analysis is now described. To analyze conversational context, the following steps are taken: 1) obtain text (e.g., receive a question) and perform translations; 2) understand concepts, entities, intents, and micro-theory; 3) relate and search; 4) ascertain the existence of related concepts; 5) logically frame concepts or needs; 6) understand the questions that can be answered from available data; and 7) answer the question. Each of the foregoing steps is discussed next, in turn.

Step 1: Obtain Text/Question and Perform Translations

In various embodiments, the cognitive intelligence platform 102 (FIG. 1) receives a text or question and performs translations as appropriate. The cognitive intelligence platform 102 supports various methods of input including text received from a touch interface (e.g., options presented in a microsurvey), text input through a microphone (e.g., words spoken into the user device), and text typed on a keyboard or on a graphical user interface. Additionally, the cognitive intelligence platform 102 supports multiple languages and auto translation (e.g., from English to Traditional/Simplified Chinese or vice versa).

The example text below is used to described methods in accordance with various embodiments herein:
“One day in January 1913. G. H. Hardy, a famous Cambridge University mathematician received a letter from an Indian named Srinivasa Ramanujan asking him for his opinion of 120 mathematical theorems that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known. Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, through born in poor and ill-paid accountant's family 100 years ago, has inspired many Indians to adopt mathematics as career.
Much of Ramanujan's work is in number theory, a branch of mathematics that deals with the subtle laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

The cognitive intelligence platform 102 analyzes the example text above to detect structural elements within the example text (e.g., paragraphs, sentences, and phrases). In some embodiments, the example text is compared to other sources of text such as dictionaries, and other general fact databases (e.g., Wikipedia) to detect synonyms and common phrases present within the example text.

Step 2: Understand Concept, Entity, Intent, and Micro-Theory

In step 2, the cognitive intelligence platform 102 parses the text to ascertain concepts, entities, intents, and micro-theories. An example output after the cognitive intelligence platform 102 initially parses the text is shown below, where concepts, and entities are shown in bold.
“One day in January 1913. G. H. Hardy, a famous Cambridge University mathematician received a letter from an Indian named Srinivasa Ramanujan asking him for his opinion of 120 mathematical theorems that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known. Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, through born in poor and ill-paid accountant's family 100 years ago, has inspired many Indians to adopt mathematics as career.
Much of Ramanujan's work is in number theory, a branch of mathematics that deals with the subtle laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

For example, the cognitive intelligence platform 102 ascertains that Cambridge is a university—which is a full understanding of the concept. The cognitive intelligence platform (e.g., the cognitive agent 110) understands what humans do in Cambridge, and an example is described below in which the cognitive intelligence platform 102 performs steps to understand a concept.

For example, in the context of the above example, the cognitive agent 110 understands the following concepts and relationships:

Cambridge employed John Edensor Littlewood (1)

Cambridge has the position Ramanujan's position at Cambridge University (2)

Cambridge employed G. H. Hardy. (3)

The cognitive agent 110 also assimilates other understandings to enhance the concepts, such as:

Cambridge has Trinity College as a suborganization. (4)

Cambridge is located in Cambridge. (5)

Alan Turing is previously enrolled at Cambridge. (6)

Stephen Hawking attended Cambridge. (7)

The statements (1)-(7) are not picked at random. Instead the cognitive agent 110 dynamically constructs the statements (1)-(7) from logic or logical inferences based on the example text above. Formally, the example statements (1)-(7) are captured as follows:

(#$subOrganizations #$UniversityOfCambridge #$TrinityCollege-Cambridge-England) (8)

(#$placeInCity #$UniversityOfCambridge #$Cityof CambridgeEngland) (9)

(#$schooling #$AlanTuring #$UniversityOfCambridge #$PreviouslyEnrolled) (10)

(#$hasAlumni # $University Of Cambridge # $ StephenHawking) (11)

Step 3: Relate and Search

Next, in step 3, the cognitive agent 110 relates various entities and topics and follows the progression of topics in the example text. Relating includes the cognitive agent 110 understanding the different instances of Hardy are all the same person, and the instances of Hardy are different from the instances of Littlewood. The cognitive agent 110 also understands that the instances Hardy and Littlewood share some similarities—e.g., both are mathematicians and they did some work together at Cambridge on Number Theory. The ability to track this across the example text is referred to as following the topic progression with a context.

Step 4: Ascertain the Existence of Related Concepts

Next, in Step 4, the cognitive agent 110 asserts non-existent concepts or relations to form new knowledge. Step 4 is an optional step for analyzing conversational context. Step 4 enhances the degree to which relationships are understood or different parts of the example text are understood together. If two concepts appear to be separate—e.g., a relationship cannot be graphically drawn or logically expressed between enough sets of concepts—there is a barrier to understanding. The barriers are overcome by expressing additional relationships. The additional relationships can be discovered using strategies like adding common sense or general knowledge sources (e.g., using the common sense data 208) or adding in other sources including a lexical variant database, a dictionary, and a thesaurus.

One example of concept progression from the example text is as follows: the cognitive agent 110 ascertains the phrase “theorems that Ramanujan said he had discovered” is related to the phrase “his results”, which is related to “Ramanujan's work is in number theory, a branch of mathematics that deals with the subtle laws and relationships that govern numbers.”

Step 5: Logically Frame Concepts or Needs

In Step 5, the cognitive agent 110 determines missing parameters—which can include for example, missing entities, missing elements, and missing nodes—in the logical framework (e.g., with a respective micro-theory). The cognitive agent 110 determines sources of data that can inform the missing parameters. Step 5 can also include the cognitive agent 110 adding common sense reasoning and finding logical paths to solutions.

With regards to the example text, some common sense concepts include:

Mathematicians develop Theorems. (12)

Theorems are hard to comprehend. (13)

Interpretations are not apparent for years. (14)

Applications are developed over time. (15)

Mathematicians collaborate and assess work. (16)

With regards to the example text, some passage concepts include:

Ramanujan did Theorems in Early 20^{th} Century. (17)

Hardy assessed Ramanujan's Theorems. (18)

Hardy collaborated with Littlewood. (19)

Hardy and Littlewood assessed Ramanujan's work (20)

Within the micro-theory of the passage analysis, the cognitive agent 110 understands and catalogs available paths to answer questions. In Step 5, the cognitive agent 110 makes the case that the concepts (12)-(20) are expressed together.

Step 6: Understand the Questions that can be Answered from Available Data

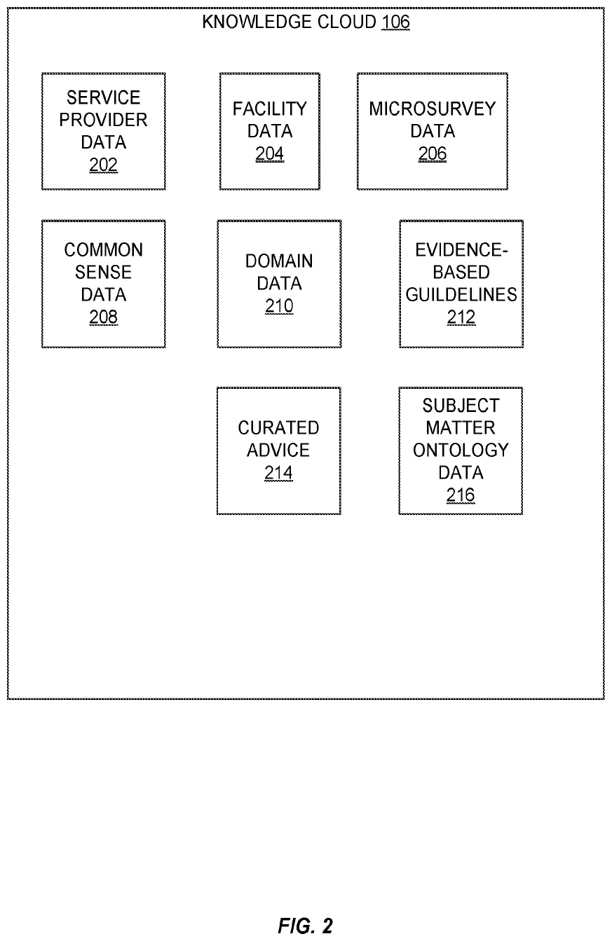

In Step 6, the cognitive agent 110 parses sub-intents and entities. Given the example text, the following questions are answerable from the cognitive agent's developed understanding of the example text, where the understanding was developed using information and context ascertained from the example text as well as the common sense data 208 (FIG. 2):

What situation causally contributed to Ramanujan's position at Cambridge? (21)

Does the author of the passage regret that Ramanujan died prematurely? (22)

Does the author of the passage believe that Ramanujan is a mathematical genius? (23)

Based on the information that is understood by the cognitive agent 110, the questions (21)-(23) can be answered.

By using an exploration method such as random walks, the cognitive agent 110 makes a determination as the paths that are plausible and reachable with the context (e.g., micro-theory) of the example text. Upon explorations, the cognitive agent 110 catalogs a set of meaningful questions. The set of meaningful questions are not asked, but instead explored based on the cognitive agent's understanding of the example text.

Given the example text, an example of exploration that yields a positive result is: “a situation X that caused Ramanujan's position.” In contrast, an example of exploration that causes irrelevant results is: “a situation Y that caused Cambridge.” The cognitive agent 110 is able to deduce that the latter exploration is meaningless, in the context of a micro-theory, because situations do not cause universities. Thus the cognitive agent 110 is able to deduce, there are no answers to Y, but there are answers to X.

Step 7: Answer the Question

In Step 7, the cognitive agent 110 provides a precise answer to a question. For an example question such as: “What situation causally contributed to Ramanujan's position at Cambridge?” the cognitive agent 110 generates a precise answer using the example reasoning:

HardyandLittlewoodsEvaluatingOfRamanujansWork (24)

HardyBeliefThatRamanujanIsAnExpertInMathematics (25)

HardysBeliefThatRamanujanIsAnExpertInMathematicsAndAGenius (26)

In order to generate the above reasoning statements (24)-(26), the cognitive agent 110 utilizes a solver or prover in the context of the example text's micro-theory—and associated facts, logical entities, relations, and assertions. As an additional example, the cognitive agent 110 uses a reasoning library that is optimized for drawing the example conclusions above within the fact, knowledge, and inference space (e.g., work space) that the cognitive agent 110 maintains.

By implementing the steps 1-7, the cognitive agent 110 analyzes conversational context. The described method for analyzing conversation context can also be used for recommending items in conversations streams. A conversational stream is defined herein as a technical discussion focused on specific topics. As related to described examples herein, the specific topics relate to health (e.g., diabetes). Throughout the lifetime of a conversational stream, a cognitive agent 110 collect information over may channels such as chat, voice, specialized applications, web browsers, contact centers, and the like.

By implementing the methods to analyze conversational context, the cognitive agent 110 can recommend a variety of topics and items throughout the lifetime of the conversational stream. Examples of items that can be recommended by the cognitive agent 110 include: surveys, topics of interest, local events, devices or gadgets, dynamically adapted health assessments, nutritional tips, reminders from a health events calendar, and the like.

Accordingly, the cognitive intelligence platform 102 provides a platform that codifies and takes into consideration a set of allowed actions and a set of desired outcomes. The cognitive intelligence platform 102 relates actions, the sequences of subsequent actions (and reactions), desired sub-outcomes, and outcomes, in a way that is transparent and logical (e.g., explainable). The cognitive intelligence platform 102 can plot a next best action sequence and a planning basis (e.g., health care plan template, or a financial goal achievement template), also in a manner that is explainable. The cognitive intelligence platform 102 can utilize a critical thinking engine 108 and a natural language database 122 (e.g., a linguistics and natural language understanding system) to relate conversation material to actions.

For purposes of this discussion, several examples are discussed in which conversational analysis is applied within the field of durational and whole-health management for a user. The discussed embodiments holistically address the care needs and well-being of the user during the course of his life. The methods and systems described herein can also be used in fields outside of whole-health management, including: phone companies that benefits from a cognitive agent; hospital systems or physicians groups that want to coach and educate patients; entities interested in user behavior and the outcome of physician-consumer interactions in terms of a progress of disease or risk management; entities that provide specialized services (e.g., test, therapies, clinical processes) to filter leads; and sellers, merchants, stores and big box retailers that want to understand which product to sell.

FIG. 2 shows additional details of a knowledge cloud, in accordance with various embodiments. In particular, FIG. 2 illustrates various types of data received from various sources, including service provider data 202, facility data 204, microsurvey data 206, commonsense data 208, domain data 210, evidence-based guidelines 212, subject matter ontology data 214, and curated advice 216. The types of data represented by the service provider data 202 and the facility data 204 include any type of data generated by the service provider 112 and the facility 114, and the above examples are not meant to be limiting. Thus, the example types of data are not meant to be limiting and other types of data can also be stored within the knowledge cloud 106 without departing from the scope of this disclosure.

The service provider data 202 is data provided by the service provider 112 (described in FIG. 1) and the facility data 204 is data provided by the facility 114 (described in FIG. 1). For example, the service provider data 202 includes medical records of a respective patient of a service provider 112 that is a doctor. In another example, the facility data 204 includes an attendance record of the respective patient, where the facility 114 is a gym. The microsurvey data 206 is data provided by the user device 104 responsive to questions presented in the microsurvey 116 (FIG. 1).

Common sense data 208 is data that has been identified as “common sense”, and can include rules that govern a respective concept and used as glue to understand other concepts.

Domain data 210 is data that is specific to a certain domain or subject area. The source of the domain data 210 can include digital libraries. In the healthcare industry, for example, the domain data 210 can include data specific to the various specialties within healthcare such as, obstetrics, anesthesiology, and dermatology, to name a few examples. In the example described herein, the evidence-based guidelines 212 include systematically developed statements to assist practitioner and patient decisions about appropriate health care for specific clinical circumstances.

Curated advice 214 includes advice from experts in a subject matter. The curated advice 214 can include peer-reviewed subject matter, and expert opinions. Subject matter ontology data 216 includes a set of concepts and categories in a subject matter or domain, where the set of concepts and categories capture properties and relationships between the concepts and categories.

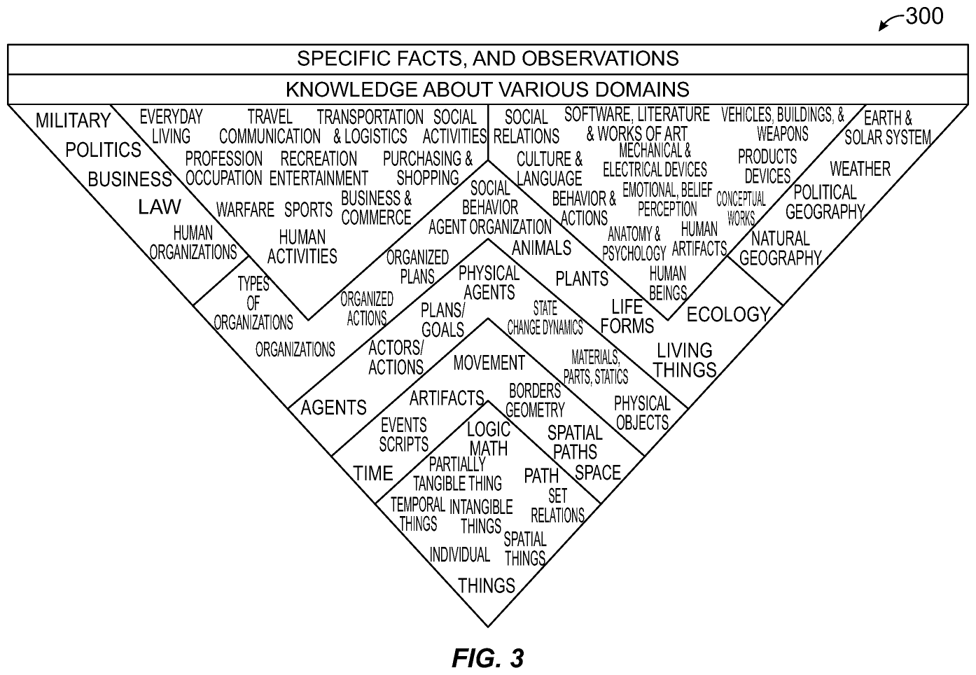

In particular, FIG. 3 illustrates an example subject matter ontology 300 that is included as part of the subject matter ontology data 216.

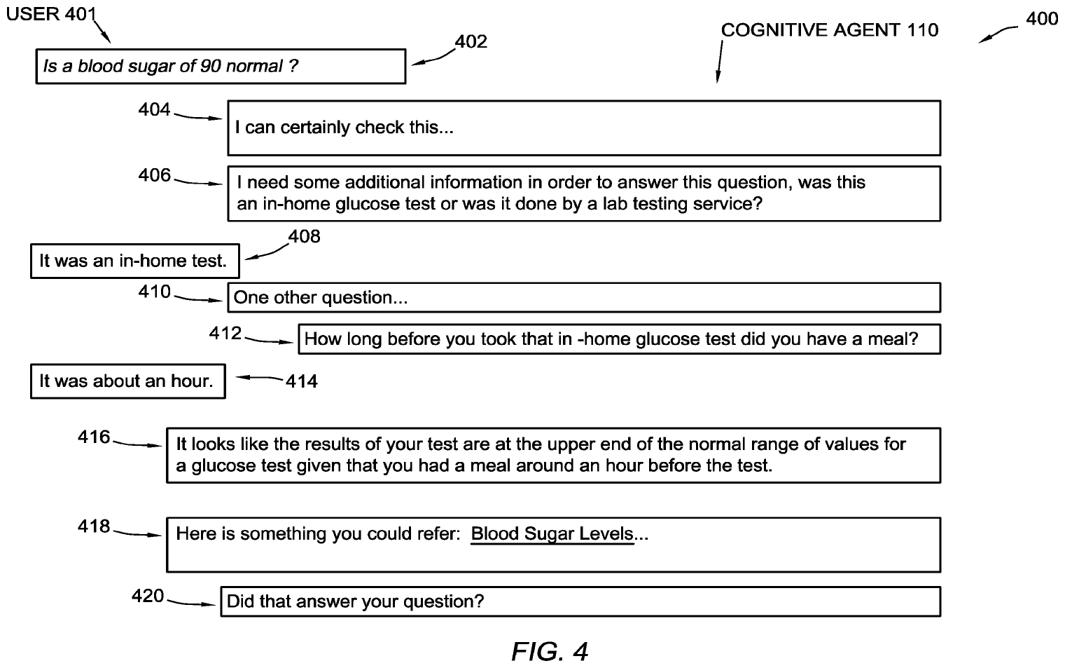

FIG. 4 illustrates aspects of a conversation 400 between a user and the cognitive intelligence platform 102, and more specifically the cognitive agent 110. For purposes of this discussion, the user 401 is a patient of the service provider 112. The user interacts with the cognitive agent 110 using a computing device, a smart phone, or any other device configured to communicate with the cognitive agent 110 (e.g., the user device 104 in FIG. 1). The user can enter text into the device using any known means of input including a keyboard, a touchscreen, and a microphone. The conversation 400 represents an example graphical user interface (GUI) presented to the user 401 on a screen of his computing device.

Initially, the user asks a general question, which is treated by the cognitive agent 110 as an “originating question.” The originating question is classified into any number of potential questions (“pursuable questions”) that are pursued during the course of a subsequent conversation. In some embodiments, the pursuable questions are identified based on a subject matter domain or goal. In some embodiments, classification techniques are used to analyze language (e.g., such as those outlined in HPS ID20180901-01 method for conversational analysis). Any known text classification technique can be used to analyze language and the originating question. For example, in line 402, the user enters an originating question about a subject matter (e.g., blood sugar) such as: “Is a blood sugar of 90 normal”? I

In response to receiving an originating question, the cognitive intelligence platform 102 (e.g., the cognitive agent 110 operating in conjunction with the critical thinking engine 108) performs a first round of analysis (e.g., which includes conversational analysis) of the originating question and, in response to the first round of analysis, creates a workspace and determines a first set of follow up questions.

In various embodiments, the cognitive agent 110 may go through several rounds of analysis executing within the workspace, where a round of analysis includes: identifying parameters, retrieving answers, and consolidating the answers. The created workspace can represent a space where the cognitive agent 110 gathers data and information during the processes of answering the originating question. In various embodiments, each originating question corresponds to a respective workspace. The conversation orchestrator 124 can assess data present within the workspace and query the cognitive agent 110 to determine if additional data or analysis should be performed.

In particular, the first round of analysis is performed at different levels, including analyzing natural language of the text, and analyzing what specifically is being asked about the subject matter (e.g., analyzing conversational context). The first round of analysis is not based solely on a subject matter category within which the originating question is classified. For example, the cognitive intelligence platform 102 does not simply retrieve a predefined list of questions in response to a question that falls within a particular subject matter, e.g., blood sugar. That is, the cognitive intelligence platform 102 does not provide the same list of questions for all questions related to the particular subject matter. Instead, for example, the cognitive intelligence platform 102 creates dynamically formulated questions, curated based on the first round of analysis of the originating question.

In particular, during the first round of analysis, the cognitive agent 110 parses aspects of the originating question into associated parameters. The parameters represent variables useful for answering the originating question. For example, the question “is a blood sugar of 90 normal” may be parsed and associated parameters may include, an age of the inquirer, the source of the value 90 (e.g., in home test or a clinical test), a weight of the inquirer, and a digestive state of the user when the test was taken (e.g., fasting or recently eaten). The parameters identify possible variables that can impact, inform, or direct an answer to the originating question.

For purposes of the example illustrated in FIG. 4, in the first round of analysis, the cognitive intelligence platform 102 inserts each parameter into the workspace associated with the originating question (line 402). Additionally, based on the identified parameters, the cognitive intelligence platform 102 identifies a customized set of follow up questions (“a first set of follow-up questions). The cognitive intelligence platform 102 inserts first set of follow-up questions in the workspace associated with the originating question.

The follow up questions are based on the identified parameters, which in turn are based on the specifics of the originating question (e.g., related to an identified micro-theory). Thus the first set of follow-up questions identified in response to, if a blood sugar is normal, will be different from a second set of follow up questions identified in response to a question about how to maintain a steady blood sugar.

After identifying the first set of follow up questions, in this example first round of analysis, the cognitive intelligence platform 102 determines which follow up question can be answered using available data and which follow-up question to present to the user. As described over the next few paragraphs, eventually, the first set of follow-up questions is reduced to a subset (“a second set of follow-up questions”) that includes the follow-up questions to present to the user.

In various embodiments, available data is sourced from various locations, including a user account, the knowledge cloud 106, and other sources. Other sources can include a service that supplies identifying information of the user, where the information can include demographics or other characteristics of the user (e.g., a medical condition, a lifestyle). For example, the service can include a doctor's office or a physical therapist's office.

Another example of available data includes the user account. For example, the cognitive intelligence platform 102 determines if the user asking the originating question, is identified. A user can be identified if the user is logged into an account associated with the cognitive intelligence platform 102. User information from the account is a source of available data. The available data is inserted into the workspace of the cognitive agent 110 as a first data.

Another example of available data includes the data stored within the knowledge cloud 106. For example, the available data includes the service provider data 202 (FIG. 2), the facility data 204, the microsurvey data 206, the common sense data 208, the domain data 210, the evidence-based guidelines 212, the curated advice 214, and the subject matter ontology data 216. Additionally data stored within the knowledge cloud 106 includes data generated by the cognitive intelligence platform 102, itself.

Follow up questions presented to the user (the second set of follow-up questions) are asked using natural language and are specifically formulated (“dynamically formulated question”) to elicit a response that will inform or fulfill an identified parameter. Each dynamically formulated question can target one parameter at a time. When answers are received from the user in response to a dynamically formulated question, the cognitive intelligence platform 102 inserts the answer into the workspace. In some embodiments, each of the answers received from the user and in response to a dynamically formulated question, is stored in a list of facts. Thus the list of facts include information specifically received from the user, and the list of facts is referred to herein as the second data.

With regards to the second set of follow-up questions (or any set of follow-up questions), the cognitive intelligence platform 102 calculates a relevance index, where the relevance index provides a ranking of the questions in the second set of follow-up questions. The ranking provides values indicative of how relevant a respective follow-up question is to the originating question. To calculate the relevance index, the cognitive intelligence platform 102 can use conversations analysis techniques described in HPS ID20180901-01_method. In some embodiments, the first set or second set of follow up questions is presented to the user in the form of the microsurvey 116.

In this first round of analysis, the cognitive intelligence platform 102 consolidates the first and second data in the workspace and determines if additional parameters need to be identified, or if sufficient information is present in the workspace to answer the originating question. In some embodiments, the cognitive agent 110 (FIG. 1) assesses the data in the workspace and queries the cognitive agent 110 to determine if the cognitive agent 110 needs more data in order to answer the originating question. The conversation orchestrator 124 executes as an interface

For a complex originating question, the cognitive intelligence platform 102 can go through several rounds of analysis. For example, in a first round of analysis the cognitive intelligence platform 102 parses the originating question. In a subsequent round of analysis, the cognitive intelligence platform 102 can create a sub question, which is subsequently parsed into parameters in the subsequent round of analysis. The cognitive intelligence platform 102 is smart enough to figure out when all information is present to answer an originating question without explicitly programming or pre-programming the sequence of parameters that need to be asked about.

In some embodiments, the cognitive agent 110 is configured to process two or more conflicting pieces of information or streams of logic. That is, the cognitive agent 110, for a given originating question can create a first chain of logic and a second chain of logic that leads to different answers. The cognitive agent 110 has the capability to assess each chain of logic and provide only one answer. That is, the cognitive agent 110 has the ability to process conflicting information received during a round of analysis.

Additionally, at any given time, the cognitive agent 110 has the ability to share its reasoning (chain of logic) to the user. If the user does not agree with an aspect of the reasoning, the user can provide that feedback which results in affecting change in a way the critical thinking engine 108 analyzed future questions and problems.

Subsequent to determining enough information is present in the workspace to answer the originating question, the cognitive agent 110 answers the question, and additionally can suggest a recommendation or a recommendation (e.g., line 418). The cognitive agent 110 suggests the reference or the recommendation based on the context and questions being discussed in the conversation (e.g., conversation 400). The reference or recommendation serves as additional handout material to the user and is provided for informational purposes. The reference or recommendation often educates the user about the overall topic related to the originating question.

In the example illustrated in FIG. 4, in response to receiving the originating questions (line 402), the cognitive intelligence platform 102 (e.g., the cognitive agent 110 in conjunction with the critical thinking engine 108) parses the originating question to determine at least one parameter: location. The cognitive intelligence platform 102 categorizes this parameter, and a corresponding dynamically formulated question in the second set of follow-up questions. Accordingly, in lines 404 and 406, the cognitive agent 110 responds by notifying the user “I can certainly check this . . . ” and asking the dynamically formulated question “I need some additional information in order to answer this question, was this an in-home glucose test or was it done by a lab or testing service?”

The user 401 enters his answer in line 408: “It was an in-home test,” which the cognitive agent 110 further analyzes to determine additional parameters: e.g., a digestive state, where the additional parameter and a corresponding dynamically formulated question as an additional second set of follow-up questions. Accordingly, the cognitive agent 110 poses the additional dynamically formulated question in lines 410 and 412: “One other question . . . ” and “How long before you took that in-home glucose test did you have a meal?” The user provides additional information in response “it was about an hour” (line 414).

The cognitive agent 110 consolidates all the received responses using the critical thinking engine 108 and the knowledge cloud 106 and determines an answer to the initial question posed in line 402 and proceeds to follow up with a final question to verify the user's initial question was answered. For example, in line 416, the cognitive agent 110 responds: “It looks like the results of your test are at the upper end of the normal range of values for a glucose test given that you had a meal around an hour before the test.” The cognitive agent 110 provides additional information (e.g., provided as a link): “Here is something you could refer,” (line 418), and follows up with a question “Did that answer your question?” (line 420).

As described above, due to the natural language database 108, in various embodiments, the cognitive agent 110 is able to analyze and respond to questions and statements made by a user 401 in natural language. That is, the user 401 is not restricted to using certain phrases in order for the cognitive agent 110 to understand what a user 401 is saying. Any phrasing, similar to how the user would speak naturally can be input by the user and the cognitive agent 110 has the ability to understand the user.

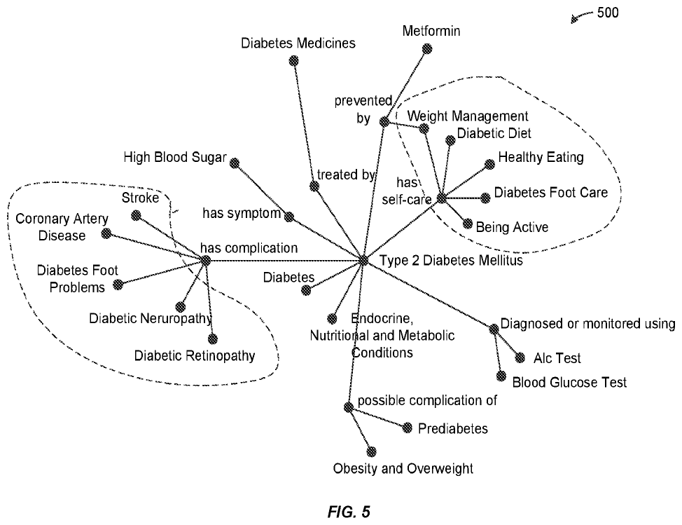

FIG. 5 illustrates a cognitive map or “knowledge graph” 500, in accordance with various embodiments. In particular, the knowledge graph represents a graph traversed by the cognitive intelligence platform 102, when assessing questions from a user with Type 2 diabetes. Individual nodes in the knowledge graph 500 represent a health artifact or relationship that is gleaned from direct interrogation or indirect interactions with the user (by way of the user device 104).

In one embodiment, the cognitive intelligence platform 102 identified parameters for an originating question based on a knowledge graph illustrated in FIG. 5. For example, the cognitive intelligence platform 102 parses the originating question to determine which parameters are present for the originating question. In some embodiments, the cognitive intelligence platform 102 infers the logical structure of the parameters by traversing the knowledge graph 500, and additionally, knowing the logical structure enables the cognitive agent 110 to formulate an explanation as to why the cognitive agent 110 is asking a particular dynamically formulated question.

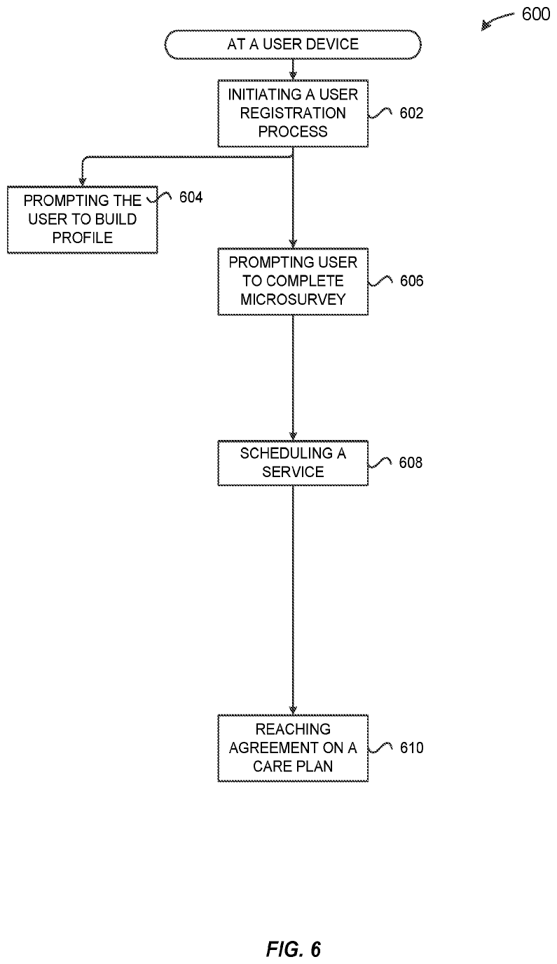

FIG. 6 shows a method, in accordance with various embodiments. The method is performed at a user device (e.g., the user device 102) and in particular, the method is performed by an application executing on the user device 102. The method begins with initiating a user registration process (block 602). The user registration can include tasks such as displaying a GUI asking the user to enter in personal information such as his name and contact information.

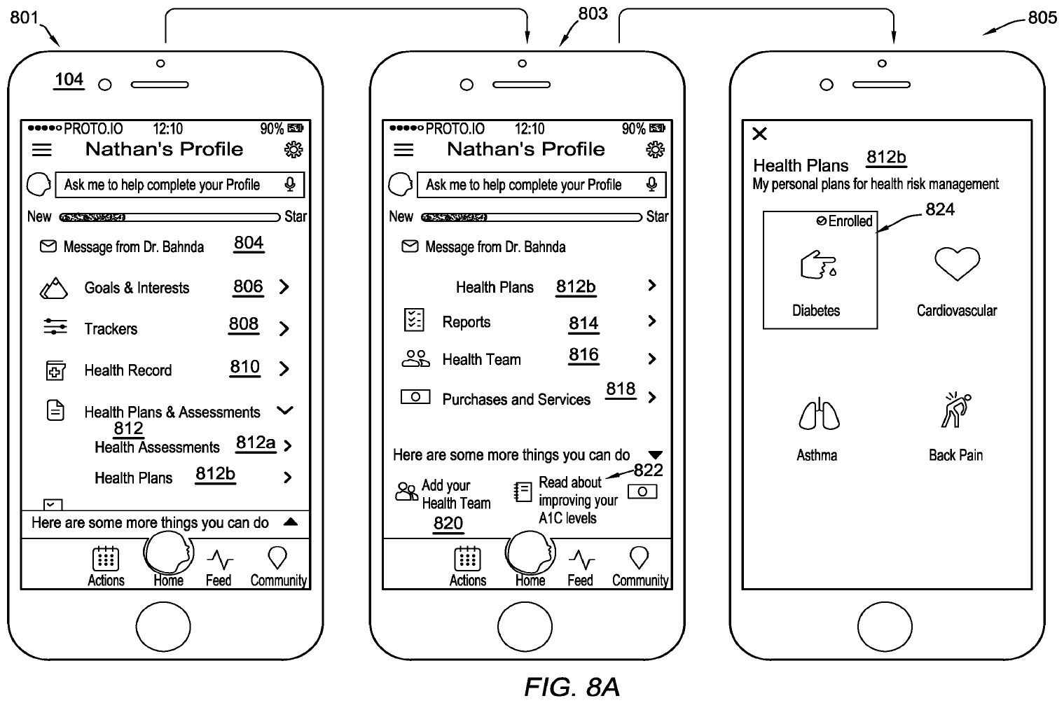

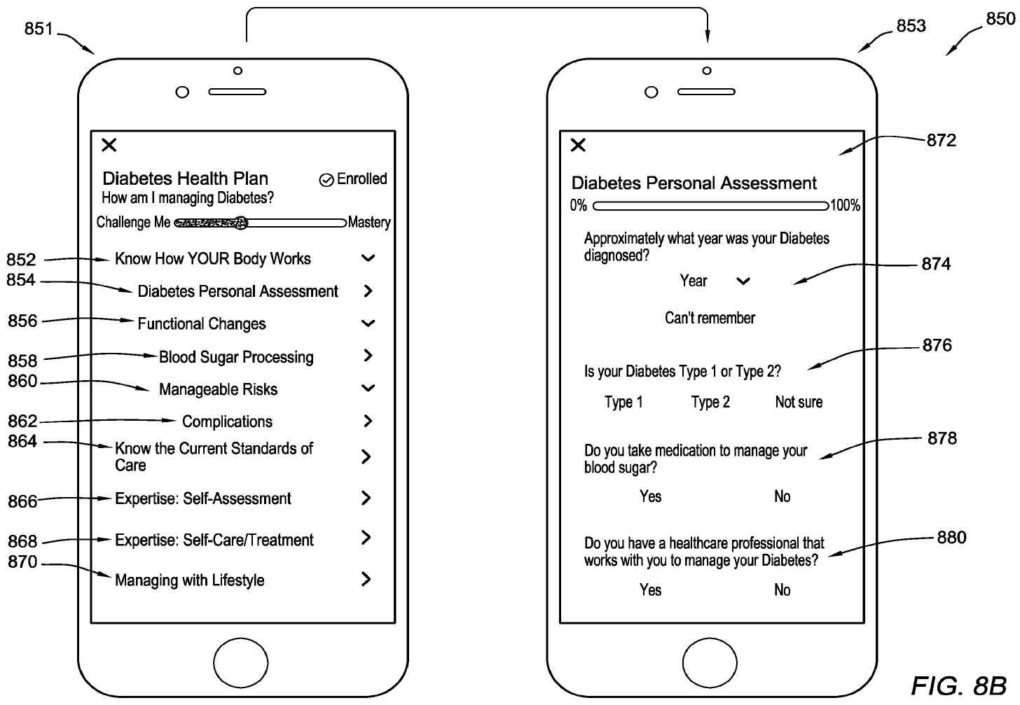

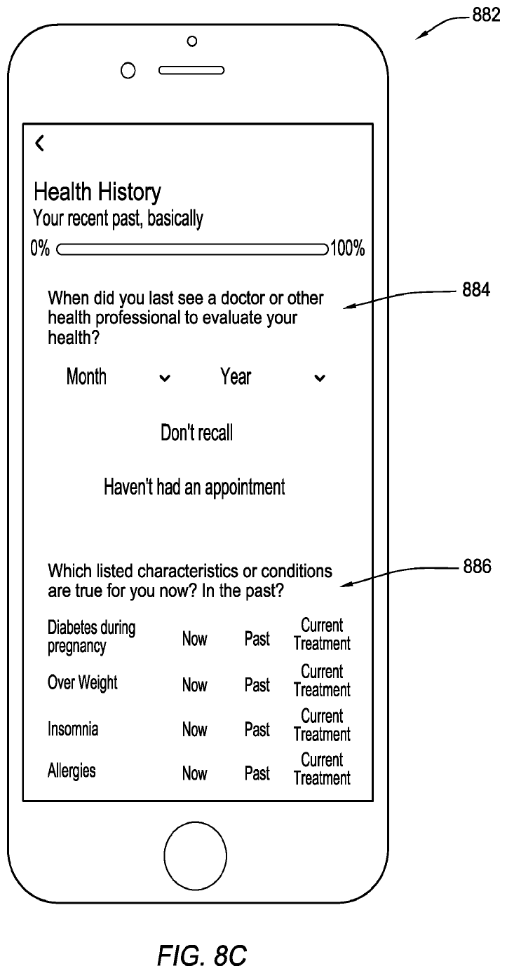

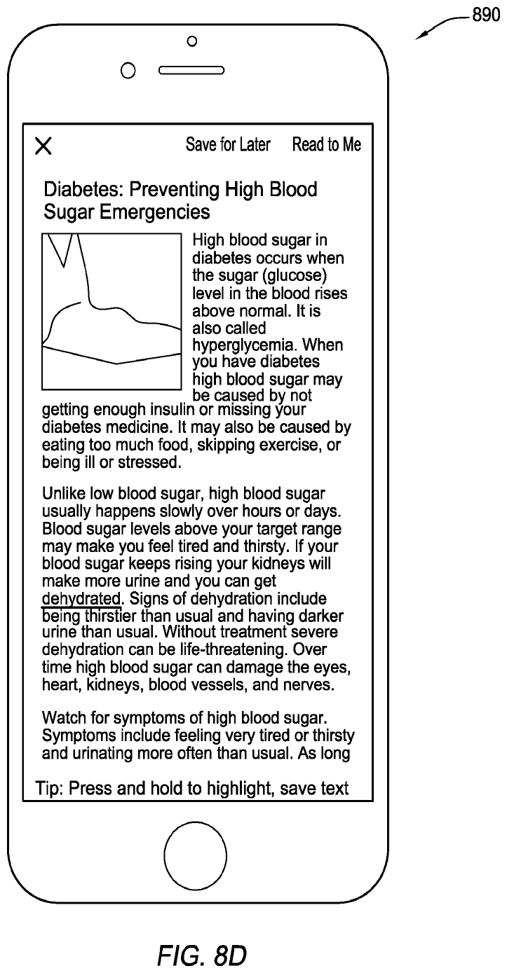

Next, the method includes prompting the user to build his profile (block 604). In various embodiments, building his profile includes displaying a GUI asking the user to enter in additional information, such as age, weight, height, and health concerns. In various embodiments, the steps of building a user profile is progressive, where building the user profile takes place over time. In some embodiments, the process of building the user profile is presented as a game. Where a user is presented with a ladder approach to create a “star profile”. Aspects of a graphical user interface presented during the profile building step are additionally discussed in FIGS. 8A-8B.

The method contemplates the build profile (block 604) method step is optional. For example, the user may complete building his profile at this method step 604, the user may complete his profile at a later time, or the cognitive intelligence platform 102 builds the user profile over time as more data about the user is received and processed. For example, the user is prompted to build his profile, however, the user fails to enter in information or skips the step. The method proceeds to prompting a user to complete a microsurvey (block 606). In some embodiments, the cognitive agent 110 uses answers received in response to the microsurvey to build the profile of the user. Overall, the data collected through the user registration process is stored and used later as available data to inform answers to missing parameters.

Next, the cognitive agent 110 proceeds to scheduling a service (block 608). The service can be scheduled such that it aligns with a health plan of the user or a protocol that results in a therapeutic goal. Next, the cognitive agent 110 proceeds to reaching agreement on a care plan (block 610).

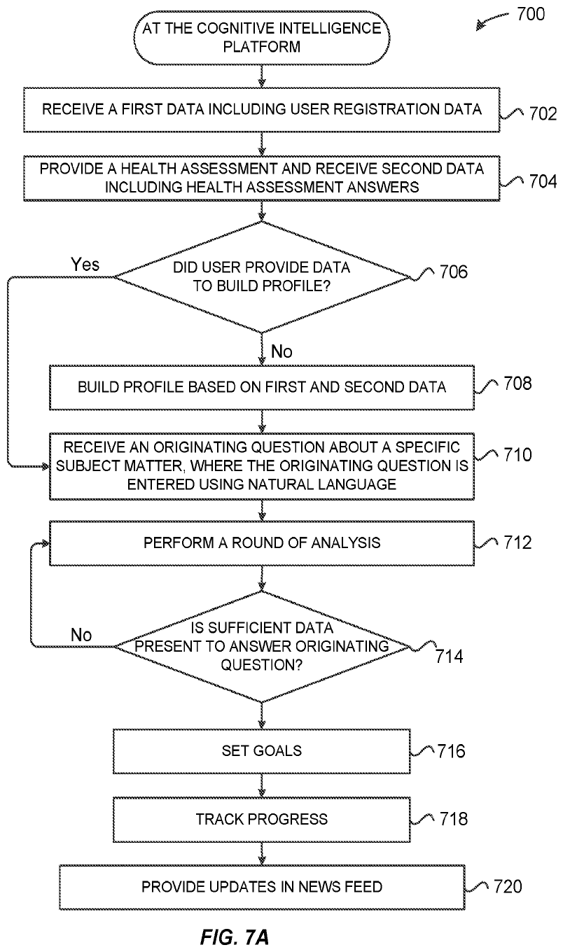

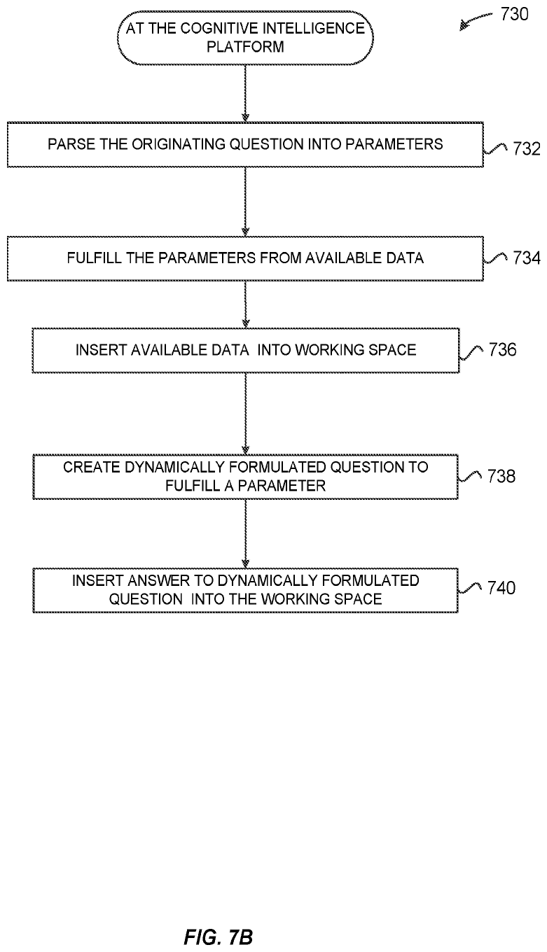

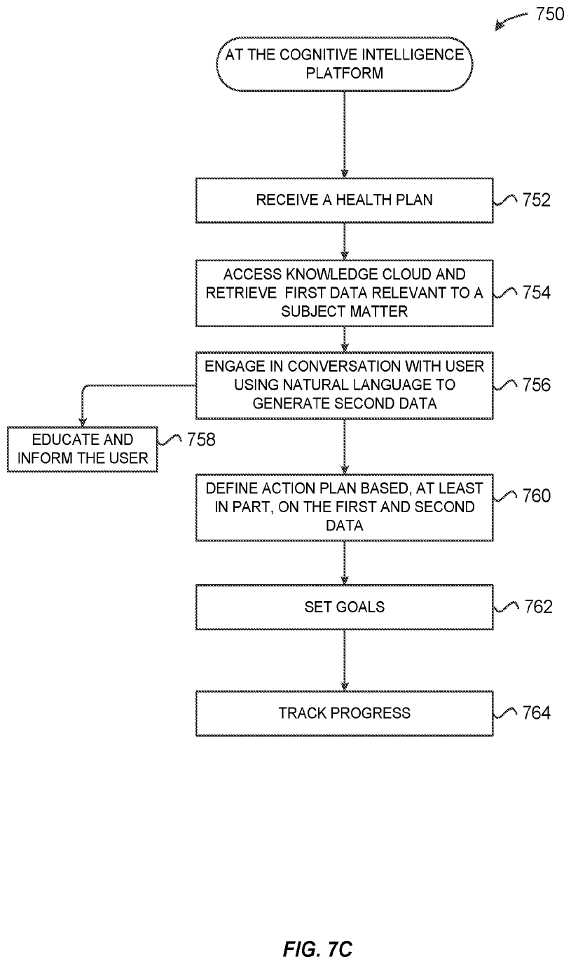

FIGS. 7A, 7B, and 7C, show methods, in accordance with various embodiments. The methods are performed at the cognitive intelligence platform. In particular, in FIG. 7A, the method begins with receiving a first data including user registration data (block 702); and providing a health assessment and receiving second data including health assessment answers (block 704). In various embodiments, the health assessment is a micro-survey with dynamically formulated questions presented to the user.

Next the method determine if the user provided data to build a profile (decision block 706). If the user did not provide data to build the profile, the method proceeds to building profile based on first and second data (block 708). If the user provided data to build the profile, the method proceeds to block 710.

At block 710, the method 700 proceeds to receiving an originating question about a specific subject matter, where the originating question is entered using natural language, and next the method proceeds to performing a round of analysis (block 712). Next, the method determines if sufficient data is present to answer originating questions (decision block 714). If no, the method proceeds to block 712 and the method performs another round of analysis. If yes, the method proceeds to setting goals (block 716), then tracking progress (block 718), and then providing updates in a news feed (block 720).

In FIG. 7B, a method 730 of performing a round of analysis is illustrated. The method begins with parsing the originating question into parameters (block 732); fulfilling the parameters from available data (block 734); inserting available data (first data) into a working space (block 736); creating a dynamically formulated question to fulfill a parameter (block 738); and inserting an answer to the dynamically formulated question into the working space (block 740).

In FIG. 7C, a method 750 is performed at the cognitive intelligence platform. The method begins with receiving a health plan (block 752); accessing the knowledge cloud and retrieving first data relevant to the subject matter (block 754); and engaging in conversation with the user using natural language to general second data (block 756). In various embodiments, the second data can include information such as a user's scheduling preferences, lifestyle choices, and education level. During the process of engaging in conversation, the method includes educating and informing the user (block 758). Next, the method includes defining an action plan based, at least in part, on the first and second data (block 760); setting goals (block 762); and tracking progress (block 764).

FIGS. 8A, 8B, 8C, and 8D illustrate aspects of interactions between a user and the cognitive intelligence platform 102, in accordance with various embodiments. As a user interacts with the GUI, the cognitive intelligence platform 102 continues to build a database of knowledge about the user based on questions asked by the user as well as answers provided by the user (e.g., available data as described in FIG. 4). In particular, FIG. 8A displays a particular screen shot 801 of the user device 104 at a particular instance in time. The screen shot 801 displays a graphical user interface (GUI) with menu items associated with a user's (e.g., Nathan) profile including Messages from the doctor (element 804), Goals (element 806), Trackers (element 808), Health Record (element 810), and Health Plans & Assessments (element 812). The menu item Health Plans & Assessments (element 812), additionally include child menu items: Health Assessments (element 812a), Health plans (812b).

The screen shot 803 displays the same GUI as in the screen shot 801, however, the user has scrolled down the menu, such that additional menu items below Health Plans & Assessments (element 812) are shown. The additional menu items include Reports (element 814), Health Team (element 816), and Purchases and Services (Element 818). Furthermore, additional menu items include Add your Health Team (element 820) and Read about improving your A1C levels (element 822).

For purposes of the example in FIG. 8A, the user selects the menu item Health Plans (element 812b). Accordingly, in response to the receiving the selection of the menu item Health Plans, types of health plans are shown, as illustrated in screen shot 805. The types of health plans shown with respect to Nathan's profile include: Diabetes (element 824), Cardiovascular, Asthma, and Back Pain. Each type of health plan leads to separate displays. For purposes of this example in FIG. 8A, the user selects the Diabetes (element 824) health plan.

In FIG. 8B, the screenshot 851 is seen in response to the user's selection of Diabetes (element 824). Example elements displayed in screenshot 851 include: Know How YOUR Body Works (element 852); Know the Current Standards of Care (element 864); Expertise: Self-Assessment (element 866); Expertise: Self-Care/Treatment (element 868); and Managing with Lifestyle (element 870). Managing with Lifestyle (element 870) focuses and tracks actions and lifestyle actions that a user can engage in. As a user's daily routine helps to manage diabetes, managing the user's lifestyle is important. The cognitive agent 110 can align a user's respective health plan based on a health assessment at enrollment. In various embodiments, the cognitive agent 110 aligns the respective health plan with an interest of the user, a goal and priority of the user, and lifestyle factors of the user—including exercise, diet and nutrition, and stress reduction.

Each of these elements 852, 864, 866, 868, and 870 can display additional sub-elements depending on a selection of the user. For example, as shown in the screen shot 851, Know How YOUR Body Works (element 852) includes additional sub-elements: Diabetes Personal Assessment (854); and Functional Changes (856). Additional sub-elements under Functional Changes (856) include: Blood Sugar Processing (858) and Manageable Risks (860). Finally, the sub-element Manageable Risks (860) includes an additional sub-element Complications (862). For purposes of this example, the user selects the Diabetes Personal Assessment (854) and the screen shot 853 shows a GUI (872) associated with the Diabetes Personal Assessment.

The Diabetes Personal Assessment includes questions such as “Approximately what year was your Diabetes diagnosed” and corresponding elements a user can select to answer including “Year” and “Can't remember” (element 874). Additional questions include “Is your Diabetes Type 1 or Type 2” and corresponding answers selectable by a user include “Type 1,” “Type 2,” and “Not sure” (element 876). Another question includes “Do you take medication to manage your blood sugar” and corresponding answers selectable by a user include “Yes” and “No” (element 878). An additional question asks “Do you have a healthcare professional that works with you to manage your Diabetes” and corresponding answers selectable by the user include “Yes” and “No” (element 880).

In various embodiments, the cognitive intelligence platform 102 collects information about the user based on responses provided by the user or questions asked by the user as the user interacts with the GUI. For example, as the user views the screen shot 851, if the user asks if diabetes is curable, this question provides information about the user such as a level of education of the user.

FIG. 8C illustrates aspects of an additional tool—e.g., a microsurvey—provided to the user that helps gather additional information about the user (e.g., available data). In various embodiments, a micro-survey represent a short targeted survey, where the questions presented in the survey are limited to a respective micro-theory. A microsurvey can be created by the cognitive intelligence platform 102 for several different purposes, including: completing a user profile, and informing a missing parameter during the process of answering an originating question.

In FIG. 8C, the microsurvey 882 gathers information related to health history, such as “when did you last see a doctor or other health professional to evaluate your health” where corresponding answers selectable by the user include specifying a month and year, “don't recall,” and “haven't had an appointment” (element 884). An additional question asks “Which listed characteristics or conditions are true for you now? In the past?” where corresponding answers selectable by the user include “Diabetes during pregnancy,” “Over Weight,” “Insomnia,” and “Allergies” (element 886). Each of the corresponding answer in element 886 also includes the option to indicate whether the characteristics or conditions are true for the user “Now”, “Past,” or “Current Treatment.”

In FIG. 8D, aspects of educating a user are shown in the screen shot 890. The screen shot displays an article titled “Diabetes: Preventing High Blood Sugar Emergencies,” and proceeds to describe when high blood sugar occurs and other information related to high blood sugar. The content displayed in the screen shot 890 is searchable and hearable as a podcast.

Accordingly, the cognitive agent 110 can answer a library of questions and provide content for many questions a user has as it related to diabetes. The information provided for purposes of educating a user is based on an overall health plan of the user, which is based on meta data analysis of interactions with the user, and an analysis of the education level of the user.

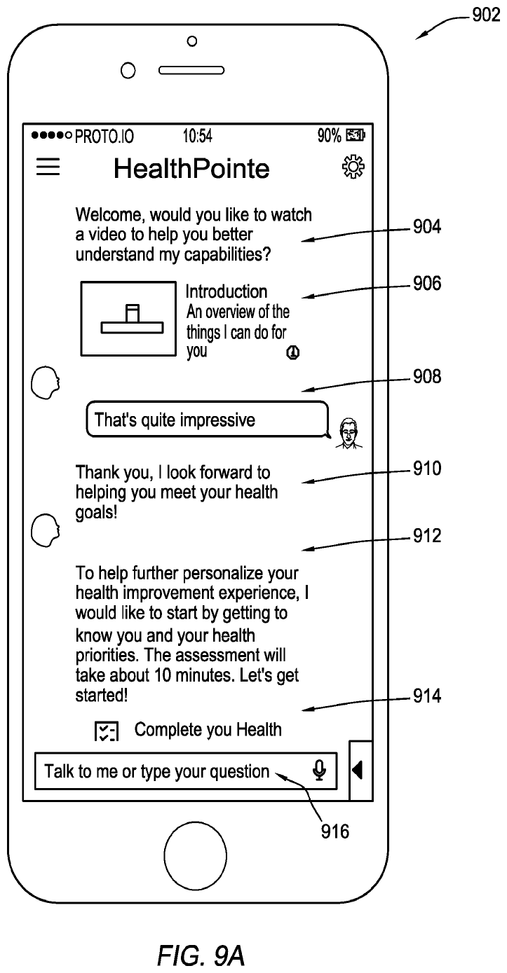

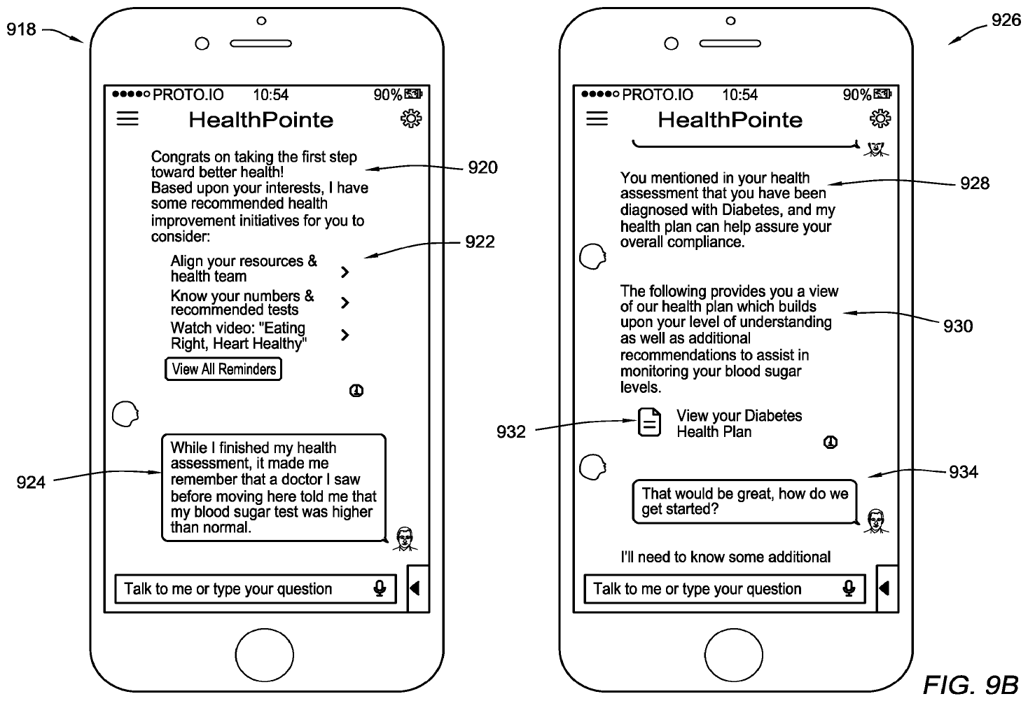

FIGS. 9A-9B illustrate aspects of a conversational stream, in accordance with various embodiments. In particular, FIG. 9A displays an example conversational stream between a user and the cognitive agent 110. The screen shot 902 is an example of a dialogue that unfolds between a user and the cognitive agent 110, after the user has registered with the cognitive intelligence platform 102. In the screen shot 902, the cognitive agent 110 begins by stating “Welcome, would you like to watch a video to help you better understand my capabilities” (element 904). The cognitive agent provides an option to watch the video (element 906). In response, the user inputs text “that's quite impressive” (element 908). In various embodiments, the user inputs text using the input box 916, which instructs the user to “Talk to me or type your question”.

Next, the cognitive agent 110 says “Thank you. I look forward to helping you meet your health goals!” (element 910). At this point, the cognitive agent 110 can probe the user for additional data by offering a health assessment survey (e.g., a microsurvey) (element 914). The cognitive agent 110 prompts the user to fill out the health assessment by stating: “To help further personalize your health improvement experience, I would like to start by getting to know you and your health priorities. The assessment will take about 10 minutes. Let's get started!” (element 912).

In FIG. 9B, an additional conversational stream between the user and the cognitive agent 110 is shown. In this example conversational stream, the user previously completed a health assessment survey. The conversational stream can follow the example conversational stream discussed in FIG. 9A.

In the screen shot 918, the cognitive agent acknowledges the user's completion of the health assessment survey (element 920) and provides additional resources to the user (element 922). In element 920, the cognitive agent states: “Congrats on taking the first step toward better health! Based upon your interest, I have some recommended health improvement initiatives for you to consider,” and presents the health improvement initiatives. In the example conversational stream, the user gets curious about a particular aspect of his health and states: “While I finished my health assessment, it made me remember that a doctor I saw before moving here told me that my blood sugar test was higher than normal.” (element 924). After receiving the statement in element 924, the cognitive agent 110 treats the statement as an originating question and undergoes an initial round of analysis (and additional rounds of analysis as needed) as described above.

The cognitive agent 110 presents an answer as shown in screen shot 926. For example, the cognitive agent 110 states: “You mentioned in your health assessment that you have been diagnosed with Diabetes, and my health plan can help assure your overall compliance” (element 928). The cognitive agent further adds: “The following provides you a view of our health plan which builds upon your level of understanding as well as additional recommendations to assist in monitoring your blood sugar levels” (element 930). The cognitive agent 110 provides the user with the option to view his Diabetes Health Plan (element 932).

The user responds “That would be great, how do we get started” (element 934). The cognitive agent 110 receives the user's response as another originated question and undergoes an initial round of analysis (and additional rounds of analysis as needed) as described above. In the example screen shot 926, the cognitive agent 110 determines additional information is needed and prompts the user for additional information.

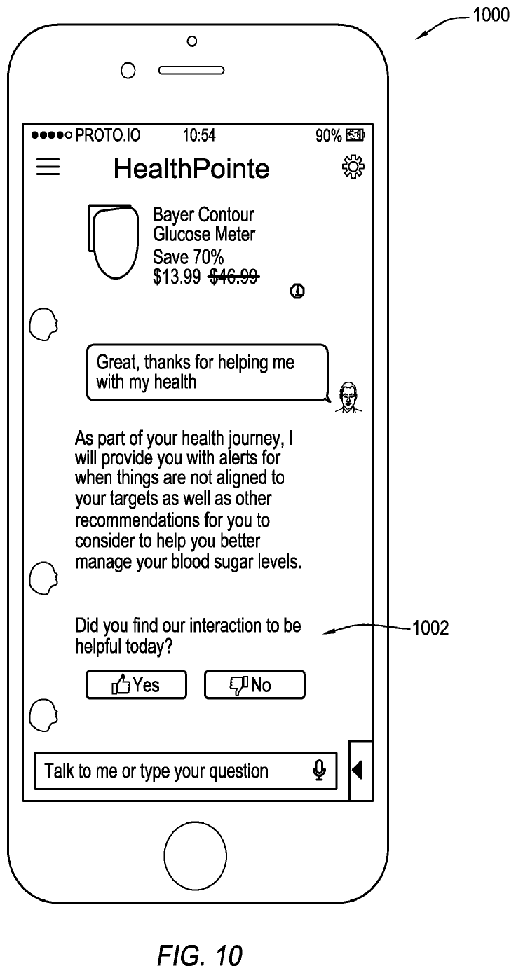

FIG. 10 illustrates an additional conversational stream, in accordance with various embodiments. In particular, in the screen shot 1000, the cognitive agent 110 elicit feedback (element 1002) to determine whether the information provided to the user was useful to the user.

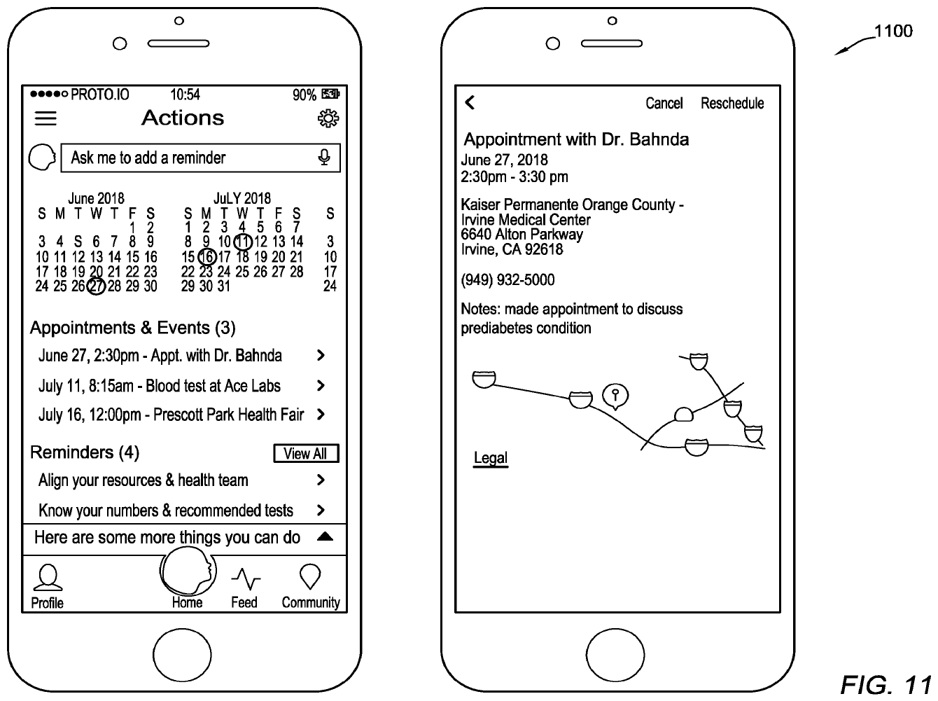

FIG. 11 illustrates aspects of an action calendar, in accordance with various embodiments. The action calendar is managed through the conversational stream between the cognitive agent 110 and the user. The action calendar aligns to care and wellness protocols, which are personalized to the risk condition or wellness needs of the user. The action calendar is also contextually aligned (e.g., what is being required or searched by the user) and hyper local (e.g., aligned to events and services provided in the local community specific to the user).

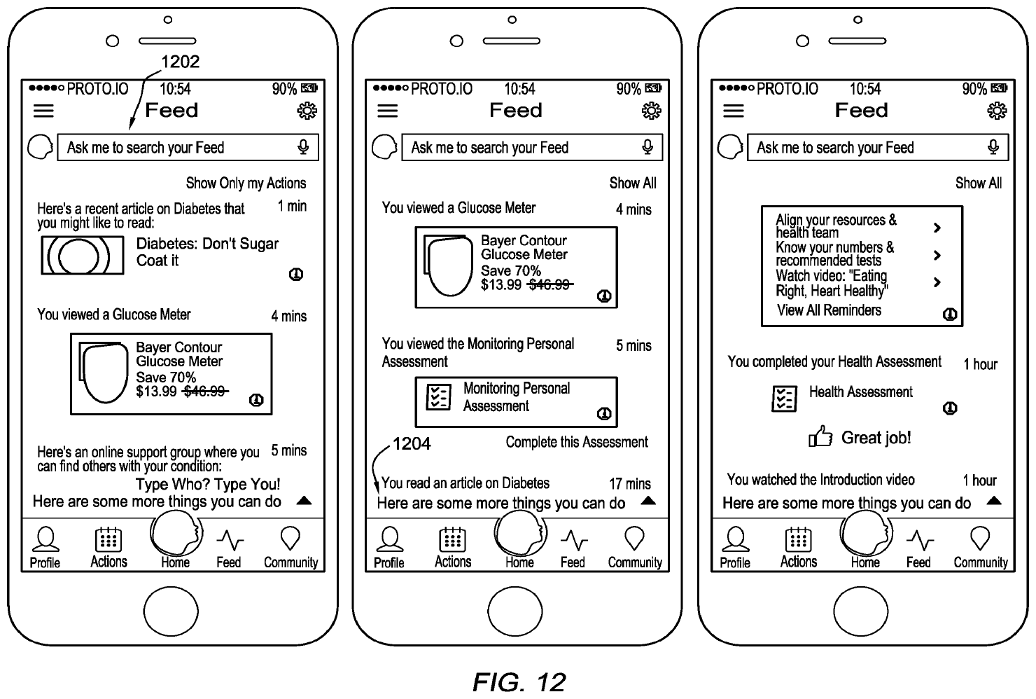

FIG. 12 illustrates aspects of a feed, in accordance with various embodiments. The feed allows a user to explore new opportunities and celebrate achieving goals (e.g., therapeutic or wellness goals). The feed provides a searchable interface (element 1202).

The feed provides an interface where the user accesses a personal log of activities the user is involved in. The personal log is searchable. For example, if the user reads an article recommended by the cognitive agent 110 and highlights passages, the highlighted passages are accessible through the search. Additionally, the cognitive agent 110 can initiate a conversational stream focused on subject matter related to the highlighted passages.

The feed provides an interface to celebrate mini achievements and successes in the user's personal goals (e.g., therapeutic or wellness goals). In the feed, the cognitive agent 110 is still available (ribbon 1204) to help search, guide, or steer the user toward a therapeutic or wellness goal.

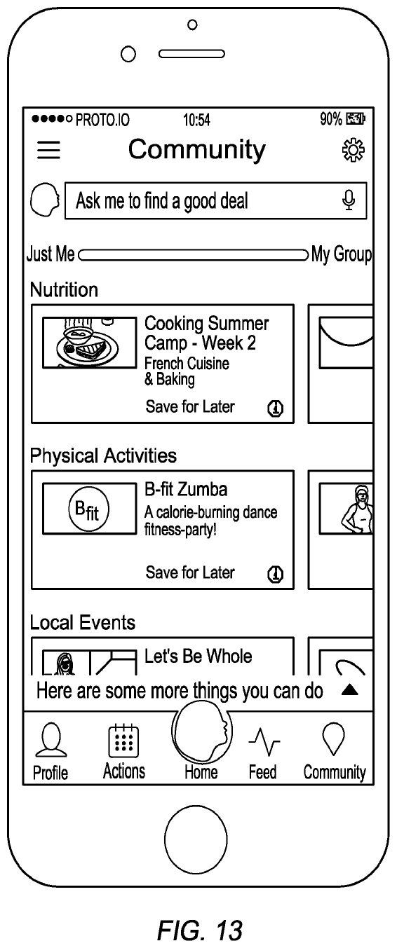

FIG. 13 illustrates aspects of a hyper-local community, in accordance with various embodiments. A hyper-local community is a digital community that is health and wellness focused and encourages the user to find opportunities for themselves and get involved in a community that is physically close to the user. The hyper-local community allows a user to access a variety of care and wellness resources within his community and example recommendations include: Nutrition; Physical Activities; Healthcare Providers; Educations; Local Events; Services; Deals and Stores; Charities; and Products offered within the community. The cognitive agent 110 optimizes suggestions which help the user progress towards a goal as opposed to providing open ended access to hyper-local assets. The recommendations are curated and monitored for relevance to the user, based on the user's goals and interactions between the user and the cognitive agent 110.

Accordingly, the cognitive intelligence platform provides several core features including:

1) the ability to identify an appropriate action plan using narrative style interactions that generates data that includes intent and causation and using narrative style interactions;

2) monitoring: integration of offline to online clinical results across the functional medicine clinical standards;

3) the knowledge cloud that includes a comprehensive knowledge base of thousands of health related topics, an educational guide to better health aligned to western and eastern culture;

4) coaching using artificial intelligence; and

5) profile and health store that offers a holistic profile of each consumers health risks and interactions, combined with a repository of services, products, lab tests, devices, deals, supplements, pharmacy & telemedicine.

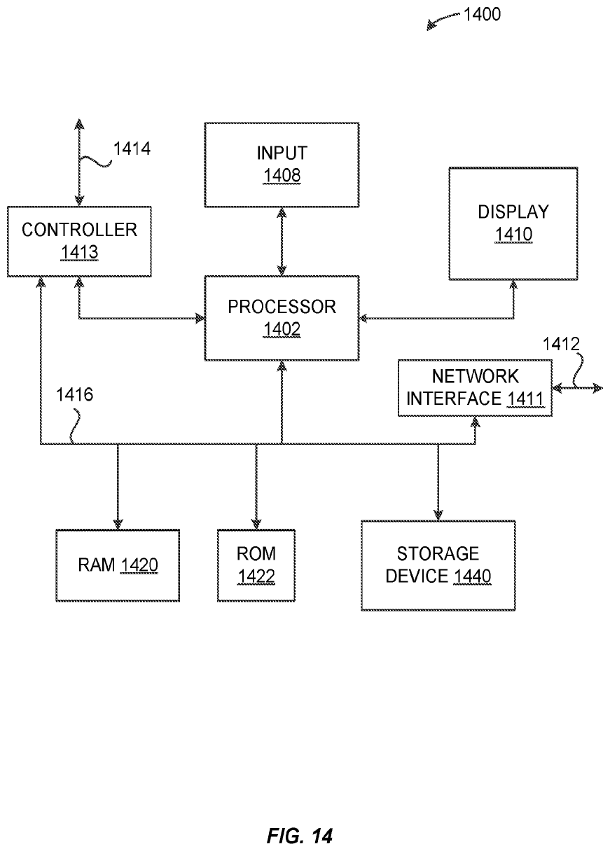

FIG. 14 illustrates a detailed view of a computing device 1400 that can be used to implement the various components described herein, according to some embodiments. In particular, the detailed view illustrates various components that can be included in the user device 104 illustrated in FIG. 1, as well as the several computing devices implementing the cognitive intelligence platform 102. The computing device 1400 may also be used by the service provider 112 and/or the facility 114. As shown in FIG. 14, the computing device 1400 can include a processor 1402 that represents a microprocessor or controller for controlling the overall operation of the computing device 1400. The computing device 1400 can also include a user input device 1408 that allows a user of the computing device 1400 to interact with the computing device 1400. For example, the user input device 1408 can take a variety of forms, such as a button, keypad, dial, touch screen, audio input interface, visual/image capture input interface, input in the form of sensor data, and so on. Still further, the computing device 1400 can include a display 1410 that can be controlled by the processor 1402 to display information to the user. A data bus 1416 can facilitate data transfer between at least a storage device 1440, the processor 1402, and a controller 1413. The controller 1413 can be used to interface with and control different equipment through an equipment control bus 1414. The computing device 1400 can also include a network/bus interface 1411 that couples to a data link 1412. In the case of a wireless connection, the network/bus interface 1411 can include a wireless transceiver.

As noted above, the computing device 1400 also includes the storage device 1440, which can comprise a single disk or a collection of disks (e.g., hard drives), and includes a storage management module that manages one or more partitions within the storage device 1440. In some embodiments, storage device 1440 can include flash memory, semiconductor (solid-state) memory or the like. The computing device 1400 can also include a Random-Access Memory (RAM) 1420 and a Read-Only Memory (ROM) 1422. The ROM 1422 can store programs, utilities or processes to be executed in a non-volatile manner. The RAM 1420 can provide volatile data storage, and stores instructions related to the operation of processes and applications executing on the computing device.

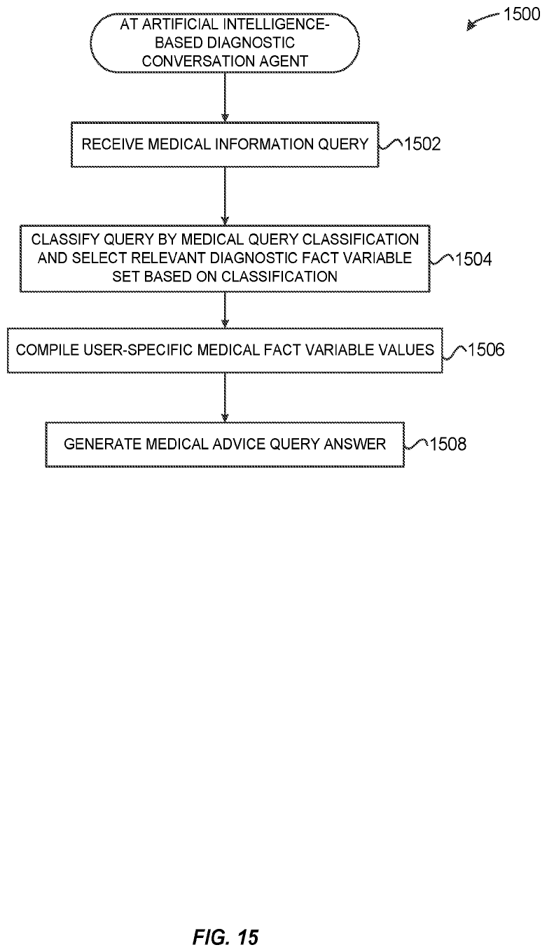

FIG. 15 shows a method (1500), in accordance with various embodiments, for answering a user-generated natural language medical information query based on a diagnostic conversational template.

In the method as shown in FIG. 15, an artificial intelligence-based diagnostic conversation agent receives a user-generated natural language medical information query as entered by a user through a user interface on a computer device (FIG. 15, block 1502). In some embodiments, the artificial intelligence-based diagnostic conversation agent is the conversation agent 110 of FIG. 1. In some embodiments the computer device is the mobile device 104 of FIG. 1. One example of a user-generated natural language medical information query as entered by a user through a user interface is the question “Is a blood sugar of 90 normal?” as shown in line 402 of FIG. 4. In some embodiments, receiving a user-generated natural language medical information query as entered by a user through a user interface on a computer device (FIG. 15, block 1502) is Step 1 as earlier discussed in the context of “Analyzing Conversational Context As Part of Conversational Analysis”.

In response to the user-generated natural language medical information query, the artificial intelligence-based diagnostic conversation agent selects a diagnostic fact variable set relevant to generating a medical advice query answer for the user-generated natural language medical information query by classifying the user-generated natural language medical information query into one of a set of domain-directed medical query classifications associated with respective diagnostic fact variable sets (FIG. 15, block 1504). In some embodiments, the artificial intelligence-based diagnostic conversation agent selecting a diagnostic fact variable set relevant to generating a medical advice query answer for the user-generated natural language medical information query by classifying the user-generated natural language medical information query into one of a set of domain-directed medical query classifications associated with respective diagnostic fact variable sets (FIG. 15, block 1504) is accomplished through one or more of Steps 2-6 as earlier discussed in the context of “Analyzing Conversational Context As Part of Conversational Analysis”.

FIG. 15 further shows compiling user-specific medical fact variable values for one or more respective medical fact variables of the diagnostic fact variable set (FIG. 15, block 1506). Compiling user-specific medical fact variable values for one or more respective medical fact variables of the diagnostic fact variable set (FIG. 15, block 1506) may include one or more of Steps 2-6 as earlier discussed in the context of “Analyzing Conversational Context As Part of Conversational Analysis”.

In response to the user-specific medical fact variable values, the artificial intelligence-based diagnostic conversation agent generates a medical advice query answer in response to the user-generated natural language medical information query (FIG. 15, block 1508). In some embodiments, this is Step 7 as earlier discussed in the context of “Analyzing Conversational Context As Part of Conversational Analysis”.

In some embodiments, compiling user-specific medical fact variable values (FIG. 15, block 1506) includes extracting a first set of user-specific medical fact variable values from a local user medical information profile associated with the user-generated natural language medical information query and requesting a second set of user specific medical fact variable values through natural-language questions sent to the user interface on the mobile device (e.g. the microsurvey data 206 of FIG. 2 that came from the microsurvey 116 of FIG. 1). The local user medical information profile can be the profile as generated in FIG. 7A at block 708.

In some embodiments, compiling user-specific medical fact variable values (FIG. 15, block 1506) includes extracting a third set of user-specific medical fact variable values that are lab result values from the local user medical information profile associated with the user generated natural language medical information query. The local user medical information profile can be the profile as generated in FIG. 7A at block 708.

In some embodiments, compiling user-specific medical fact variable values (FIG. 15, block 1506) includes extracting a fourth set of user-specific medical variable values from a remote medical data service profile associated with the local user medical information profile. The remote medical data service profile can be the service provider data 202 of FIG. 2, which can come from the service provider 112 of FIG. 1. The local user medical information profile can be the profile as generated in FIG. 7A at block 708.

In some embodiments, compiling user-specific medical fact variable values (FIG. 15, block 1506) includes extracting a fifth set of user-specific medical variable values from demographic characterizations provided by a remote data service analysis of the local user medical information profile. The remote demographic characterizations can be the service provider data 202 of FIG. 2, which can come from the service provider 112 of FIG. 1. The local user medical information profile can be the profile as generated in FIG. 7A at block 708.

In some embodiments, generating the medical advice query answer (FIG. 15, block 1508) includes providing a treatment action-item recommendation in response to user-specific medical fact values that may be non-responsive to the medical question presented in the user-generated natural language medical information query. Such an action could define an action plan based on the data compiled (FIG. 15, block 1506), as shown in FIG. 7C, block 758.

In some embodiments, generating the medical advice query answer (FIG. 15, block 1506) includes providing a medical education media resource in response to user-specific medical fact variable values that may be non-responsive to the medical question presented in the user-generated natural language medical information query. Such an action could serve to educate and inform the user, as in block 758 of FIG. 7C.

In some embodiments, selecting a diagnostic fact variable set relevant to generating a medical advice query answer for the user-generated natural language medical information query by classifying the user-generated natural language medical information query into one of a set of domain-directed medical query classifications associated with respective diagnostic fact variable sets (FIG. 15, block 1504) includes classifying the user-generated natural language medical information query into one of a set of domain-directed medical query classifications based on relevance to the local user medical information profile associated with the user-generated natural language medical information query. The local user medical information profile can be the profile as generated in FIG. 7A at block 708.

In some embodiments, the method (1500) for answering a user-generated natural language medical information query based on a diagnostic conversational template is implemented as a computer program product in a computer-readable medium.

In some embodiments, the system and method 1500 shown in FIG. 15 and described above is implemented on the computing device 1400 shown in FIG. 14.

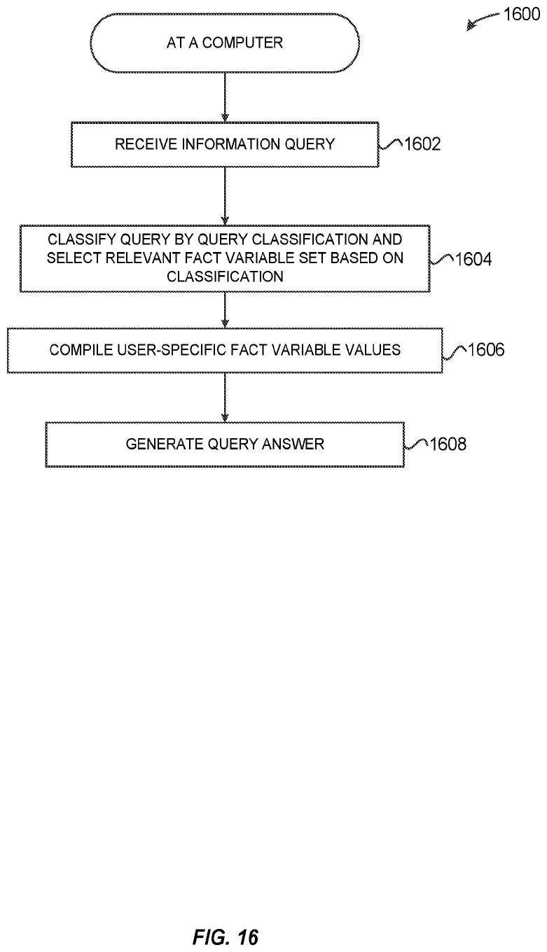

FIG. 16 shows a method (1600), in accordance with various embodiments, for answering a user-generated natural language query based on a conversational template.

In the method as shown in FIG. 16, an artificial intelligence-based conversation agent receives a user-generated natural language query as entered by a user through a user interface (FIG. 16, block 1602). In some embodiments, the artificial intelligence-based conversation agent is the conversation agent 110 of FIG. 1. In some embodiments, the user interface is on a computer device. In some embodiments the computer device is the mobile device 104 of FIG. 1. One example of a user-generated natural language query as entered by a user through a user interface is the question “Is a blood sugar of 90 normal?” as shown in line 402 of FIG. 4. In some embodiments, receiving a user-generated natural language query as entered by a user through a user interface on a computer device (FIG. 16, block 1602) is Step 1 as earlier discussed in the context of “Analyzing Conversational Context As Part of Conversational Analysis”.

In response to the user-generated natural language query, the artificial intelligence-based conversation agent selects a fact variable set relevant to generating a query answer for the user-generated natural language query by classifying the user-generated natural language query into one of a set of domain-directed query classifications associated with respective fact variable sets (FIG. 16, block 1604). In some embodiments, the artificial intelligence-based conversation agent selecting a fact variable set relevant to generating a query answer for the user-generated natural language query by classifying the user-generated natural language query into one of a set of domain-directed query classifications associated with respective fact variable sets (FIG. 16, block 1604) is accomplished through one or more of Steps 2-6 as earlier discussed in the context of “Analyzing Conversational Context As Part of Conversational Analysis”.

FIG. 16 further shows compiling user-specific variable values for one or more respective fact variables of the fact variable set (FIG. 16, block 1606). Compiling user-specific fact variable values for one or more respective fact variables of the fact variable set (FIG. 16, block 1606) may include one or more of Steps 2-6 as earlier discussed in the context of “Analyzing Conversational Context As Part of Conversational Analysis”.

In response to the user-specific fact variable values, the artificial intelligence-based conversation agent generates a query answer in response to the user-generated natural language query (FIG. 16, block 1608). In some embodiments, this is Step 7 as earlier discussed in the context of “Analyzing Conversational Context As Part of Conversational Analysis”.

In some embodiments, compiling user-specific fact variable values (FIG. 16, block 1606) includes extracting a first set of user-specific fact variable values from a local user profile associated with the user-generated natural language query and requesting a second set of user specific variable values through natural-language questions sent to the user interface on the mobile device (e.g. the microsurvey data 206 of FIG. 2 that came from the microsurvey 116 of FIG. 1). The local user profile can be the profile as generated in FIG. 7A at block 708. In some embodiments, the natural language questions sent to the user interface on the mobile device can be a part of a conversation template.

In some embodiments, compiling user-specific fact variable values (FIG. 16, block 1606) includes extracting a third set of user-specific fact variable values that are test result values from the local user profile associated with the user generated natural language query. The local user profile can be the profile as generated in FIG. 7A at block 708. In some embodiments, compiling user-specific fact variable values (FIG. 16, block 1606) includes extracting a fourth set of user-specific variable values from a remote data service profile associated with the local user profile. The remote data service profile can be the service provider data 202 of FIG. 2, which can come from the service provider 112 of FIG. 1. The local user profile can be the profile as generated in FIG. 7A at block 708.

In some embodiments, compiling user-specific fact variable values (FIG. 16, block 1606) includes extracting a fifth set of user-specific variable values from demographic characterizations provided by a remote data service analysis of the local user profile. The remote demographic characterizations can be the service provider data 202 of FIG. 2, which can come from the service provider 112 of FIG. 1. The local user profile can be the profile as generated in FIG. 7A at block 708.

In some embodiments, generating the query answer (FIG. 16, block 1608) includes providing a action-item recommendation in response to user-specific fact values that may be non-responsive to the question presented in the user-generated natural language query. Such an action could define an action plan based on the data compiled (FIG. 16, block 1606), as shown in FIG. 7C, block 758.

In some embodiments, generating the advice query answer (FIG. 16, block 1606) includes providing an education media resource in response to user-specific fact variable values that may be non-responsive to the question presented in the user-generated natural language query. Such an action could serve to educate and inform the user, as in block 758 of FIG. 7C.

In some embodiments, selecting a fact variable set relevant to generating a query answer for the user-generated natural language query by classifying the user-generated natural language query into one of a set of domain-directed query classifications associated with respective fact variable sets (FIG. 16, block 1604) includes classifying the user-generated natural language query into one of a set of domain-directed query classifications based on relevance to the local user profile associated with the user-generated natural language query. The local user profile can be the profile as generated in FIG. 7A at block 708.

In some embodiments, the method (1600) for answering a user-generated natural language query based on a conversational template is implemented as a computer program product in a computer-readable medium.

In some embodiments, the system and method shown in FIG. 16 and described above is implemented in the cognitive intelligence platform 102 shown in FIG. 1.

In the cognitive intelligence platform 102, a cognitive agent 110 is configured for receiving a user-generated natural language query at an artificial intelligence-based conversation agent from a user interface on a user device 104 (FIG. 16, block 1602).

A critical thinking engine 108 is configured for, responsive to content of the user-generated natural language query, selecting a fact variable set relevant to generating a query answer for the user-generated natural language query by classifying the user-generated natural language query into one of a set of domain-directed query classifications associated with respective fact variable sets (FIG. 16, block 1604).

Included is a knowledge cloud 106 that compiles user-specific fact variable values for one or more respective fact variables of the fact variable set (FIG. 16, block 1606).

Responsive to the fact variable values, the cognitive agent 110 is further configured for generating the query answer in response to the user-generated natural language query (FIG. 16, block 1606).

In some embodiments, the system and method 1600 shown in FIG. 16 and described above is implemented on the computing device 1400 shown in FIG. 14.

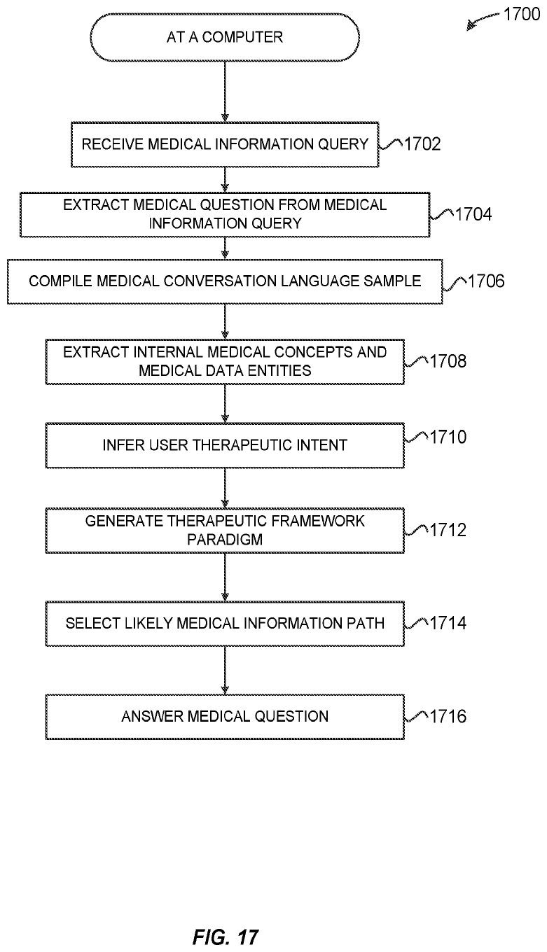

FIG. 17 shows a computer-implemented method 1700 for answering natural language medical information questions posed by a user of a medical conversational interface of a cognitive artificial intelligence system. In some embodiments, the method 1700 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 1400 shown in FIG. 14.

The method 1700 involves receiving a user-generated natural language medical information query from a medical conversational user interface at an artificial intelligence-based medical conversation cognitive agent (block 1702). In some embodiments, receiving a user-generated natural language medical information query from a medical conversational user interface at an artificial intelligence-based medical conversation cognitive agent (block 1702) is performed by a cognitive agent that is a part of the cognitive intelligence platform and is configured for this purpose. In some embodiments, the artificial intelligence-based diagnostic conversation agent is the conversation agent 110 of FIG. 1. One example of a user-generated natural language medical information query is “Is a blood sugar of 90 normal?” as shown in line 402 of FIG. 4. In some embodiments, the user interface is on the mobile device 104 of FIG. 1. In some embodiments, receiving a user-generated natural language medical information query from a medical conversational user interface at an artificial intelligence-based medical conversation cognitive agent (block 1702) is Step 1 as earlier discussed in the context of “Analyzing Conversational Context As Part of Conversational Analysis”.

The method 1700 further includes extracting a medical question from a user of the medical conversational user interface from the user-generated natural language medical information query (block 1704). In some embodiments, extracting a medical question from a user of the medical conversational user interface from the user-generated natural language medical information query (block 1704) is performed by a critical thinking engine configured for this purpose. In some embodiments, the critical thinking engine is the critical thinking engine 108 of FIG. 1. In some embodiments, extracting a medical question from a user of the medical conversational user interface from the user-generated natural language medical information query (block 1704) is accomplished through one or more of Steps 2-6 as earlier discussed in the context of “Analyzing Conversational Context As Part of Conversational Analysis”.

The method 1700 includes compiling a medical conversation language sample (block 1706). In some embodiments, compiling a medical conversation language sample (block 1706) is performed by a critical thinking engine configured for this purpose. In some embodiments, the critical thinking engine is the critical thinking engine 108 of FIG. 1. The medical conversation language sample can include items of health-information-related-text derived from a health-related conversation between the artificial intelligence-based medical conversation cognitive agent and the user. In some embodiments compiling a medical conversation language sample (block 1706) is accomplished through one or more of Steps 2-6 as earlier discussed in the context of “Analyzing Conversational Context As Part of Conversational Analysis”.

The method 1700 involves extracting internal medical concepts and medical data entities from the medical conversation language sample (block 1708). In some embodiments, extracting internal medical concepts and medical data entities from the medical conversation language sample (block 1708) is performed by a critical thinking engine configured for this purpose. In some embodiments, the critical thinking engine is the critical thinking engine 108 of FIG. 1. The internal medical concepts can include descriptions of medical attributes of the medical data entities. In some embodiments, extracting internal medical concepts and medical data entities from the medical conversation language sample (block 1708) is accomplished through one or more of Steps 2-6 as earlier discussed in the context of “Analyzing Conversational Context As Part of Conversational Analysis”.

The method 1700 involves inferring a therapeutic intent of the user from the internal medical concepts and the medical data entities (block 1710). In some embodiments, inferring a therapeutic intent of the user from the internal medical concepts and the medical data entities (block 1710) is performed by a critical thinking engine configured for this purpose. In some embodiments, the critical thinking engine is the critical thinking engine 108 of FIG. 1. In some embodiments, inferring a therapeutic intent of the user from the internal medical concepts and the medical data entities (block 1710) is accomplished as in Step 2 as earlier discussed in the context of “Analyzing Conversational Context As Part of Conversational Analysis”.

The method 1700 includes generating a therapeutic paradigm logical framework 1800 for interpreting of the medical question (block 1712). In some embodiments, generating a therapeutic paradigm logical framework 1800 for interpreting of the medical question (block 1712) is performed by a critical thinking engine configured for this purpose. In some embodiments, the critical thinking engine is the critical thinking engine 108 of FIG. 1. In some embodiments, generating a therapeutic paradigm logical framework 1800 for interpreting of the medical question (block 1712) is accomplished as in Step 5 as earlier discussed in the context of “Analyzing Conversational Context As Part of Conversational Analysis”.

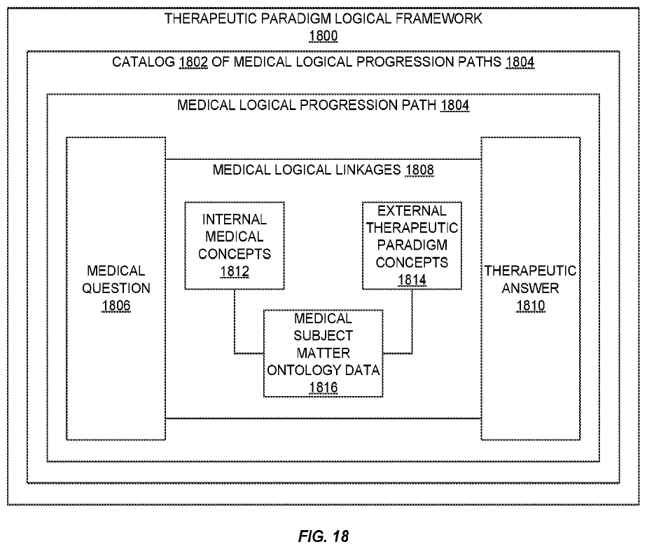

FIG. 18 shows an example therapeutic paradigm logical framework 1800. The therapeutic paradigm logical framework 1800 includes a catalog 1802 of medical logical progression paths 1804 from the medical question 1806 to respective therapeutic answers 1810.

Each of the medical logical progression paths 1804 can include one or more medical logical linkages 1808 from the medical question 1806 to a therapeutic path-specific answer 1810.

The medical logical linkages 1808 can include the internal medical concepts 1812 and external therapeutic paradigm concepts 1814 derived from a store of medical subject matter ontology data 1816. In some embodiments, the store of subject matter ontology data 1816 is contained in a knowledge cloud. In some embodiments, the knowledge cloud is the knowledge cloud 102 of FIGS. 1 and 2. In some embodiments, the subject matter ontology data 1816 is the subject matter ontology data 216 of FIG. 2. In some embodiments, the subject matter ontology data 1816 includes the subject matter ontology 300 of FIG. 3.

The method 1700 shown in FIG. 17 further includes selecting a likely medical information path from among the medical logical progression paths 1804 to a likely path-dependent medical information answer based at least in part upon the therapeutic intent of the user (block 1714). In some embodiments, selecting a likely medical information path from among the medical logical progression paths 1804 to a likely path-dependent medical information answer based at least in part upon the therapeutic intent of the user (block 1714 is performed by a critical thinking engine configured for this purpose. In some embodiments, the critical thinking engine is the critical thinking engine 108 of FIG. 1. The selection can also be based in part upon the sufficiency of medical diagnostic data to complete the medical logical linkages 1808. In some embodiments, selection can also be based in part upon the sufficiency of medical diagnostic data to complete the medical logical linkages 1808 can be performed by a critical thinking engine that is further configured for this purpose. In some embodiments, the critical thinking engine is the critical thinking engine 108 of FIG. 1. The medical diagnostic data can include user-specific medical diagnostic data. The selection can also be based in part upon treatment sub-intents including tactical constituents related to the therapeutic intent of the user by the store of medical subject matter ontology data 1816. In some embodiments, selection based in part upon treatment sub-intents including tactical constituents related to the therapeutic intent of the user by the store of medical subject matter ontology data 1816 can be performed by a critical thinking engine further configured for this purpose. In some embodiments, the critical thinking engine is the critical thinking engine 108 of FIG. 1. The selection can further occur after requesting additional medical diagnostic data from the user. An example of requesting additional medical diagnostic data from the user is shown in FIG. 4 on line 406 “I need some additional information in order to answer this question, was this an in-home glucose test or was it done by a lab or testing service”. In some embodiments, the process of selection after requesting additional medical diagnostic data from the user can be performed by a critical thinking engine further configured for this purpose. In some embodiments, the critical thinking engine is the critical thinking engine 108 of FIG. 1. In some embodiments, selecting a likely medical information path from among the medical logical progression paths 1804 to a likely path-dependent medical information answer based at least in part upon the therapeutic intent of the user (block 1714) is accomplished through one or more of Steps 5-6 as earlier discussed in the context of “Analyzing Conversational Context As Part of Conversational Analysis”.

The method 1700 involves answering the medical question by following the likely medical information path to the likely path-dependent medical information answer (block 1716). In some embodiments, answering the medical question by following the likely medical information path to the likely path-dependent medical information answer (block 1716) is performed by a critical thinking engine configured for this purpose. In some embodiments, the critical thinking engine is the critical thinking engine 108 of FIG. 1. In some embodiments, answering the medical question by following the likely medical information path to the likely path-dependent medical information answer (block 1716) is accomplished as in Step 7 as earlier discussed in the context of “Analyzing Conversational Context As Part of Conversational Analysis”.

The method 1700 can further include relating medical inference groups of the internal medical concepts. In some embodiments, relating medical inference groups of the internal medical concepts is performed by a critical thinking engine further configured for this purpose. In some embodiments, the critical thinking engine is the critical thinking engine 108 of FIG. 1. Relating medical inference groups of the internal medical concepts can be based at least in part on shared medical data entities for which each internal medical concept of a medical inference group of internal medical concepts describes a respective medical data attribute. In some embodiments, relating medical inference groups of the internal medical concepts based at least in part on shared medical data entities for which each internal medical concept of a medical inference group of internal medical concepts describes a respective medical data attribute can be performed by a critical thinking engine further configured for this purpose. In some embodiments, the critical thinking engine is the critical thinking engine 108 of FIG. 1.

In some embodiments, the method 1700 of FIG. 17 is implemented as a computer program product in a computer-readable medium.

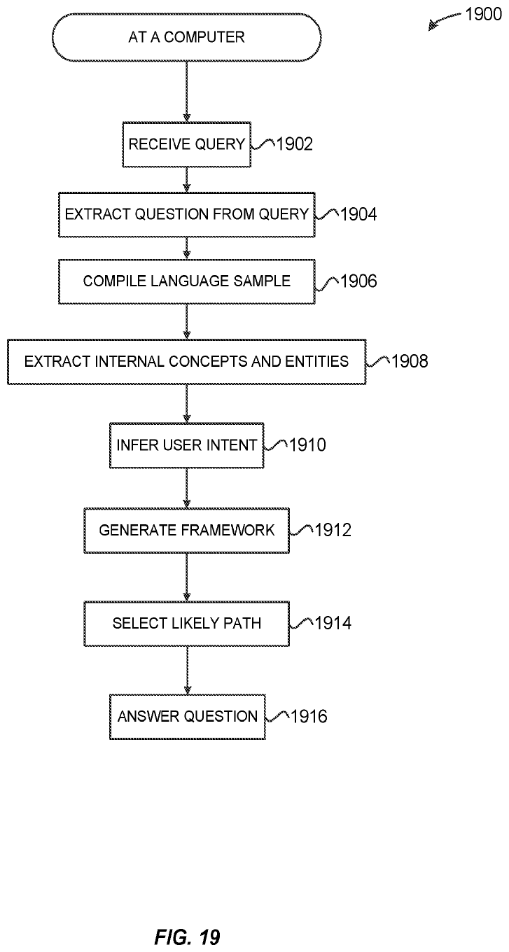

FIG. 19 shows a computer-implemented method 1900 for answering natural language questions posed by a user of a conversational interface of an artificial intelligence system. In some embodiments, the method 1900 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 1400 shown in FIG. 14.

The method 1900 involves receiving a user-generated natural language query at an artificial intelligence-based conversation agent (block 1902). In some embodiments, receiving a user-generated natural language query from a conversational user interface at an artificial intelligence-based conversation cognitive agent (block 1902) is performed by a cognitive agent that is a part of the cognitive intelligence platform and is configured for this purpose. In some embodiments, the artificial intelligence-based conversation agent is the conversation agent 110 of FIG. 1. One example of a user-generated natural language query is “Is a blood sugar of 90 normal?” as shown in line 402 of FIG. 4. In some embodiments, the user interface is on the mobile device 104 of FIG. 1. In some embodiments, receiving a user-generated natural language query from a conversational user interface at an artificial intelligence-based conversation cognitive agent (block 1902) is Step 1 as earlier discussed in the context of “Analyzing Conversational Context As Part of Conversational Analysis”.

The method 1900 further includes extracting a question from a user of the conversational user interface from the user-generated natural language query (block 1904). In some embodiments, extracting a question from a user of the conversational user interface from the user-generated natural language query (block 1904) is performed by a critical thinking engine configured for this purpose. In some embodiments, the critical thinking engine is the critical thinking engine 108 of FIG. 1. In some embodiments, extracting a question from a user of the conversational user interface from the user-generated natural language query (block 1904) is accomplished through one or more of Steps 2-6 as earlier discussed in the context of “Analyzing Conversational Context As Part of Conversational Analysis”.

The method 1900 includes compiling a language sample (block 1906). In some embodiments, compiling a language sample (block 1906) is performed by a critical thinking engine configured for this purpose. In some embodiments, the critical thinking engine is the critical thinking engine 108 of FIG. 1. The language sample can include items of health-information-related-text derived from a health-related conversation between the artificial intelligence-based conversation cognitive agent and the user. In some embodiments compiling a language sample (block 1906) is accomplished through one or more of Steps 2-6 as earlier discussed in the context of “Analyzing Conversational Context As Part of Conversational Analysis”.

The method 1900 involves extracting internal concepts and entities from the language sample (block 1908). In some embodiments, extracting internal concepts and entities from the language sample (block 1908) is performed by a critical thinking engine configured for this purpose. In some embodiments, the critical thinking engine is the critical thinking engine 108 of FIG. 1. The internal concepts can include descriptions of attributes of the entities. In some embodiments, extracting internal concepts and entities from the language sample (block 1908) is accomplished through one or more of Steps 2-6 as earlier discussed in the context of “Analyzing Conversational Context As Part of Conversational Analysis”.

The method 1900 involves inferring an intent of the user from the internal concepts and the entities (block 1910). In some embodiments, inferring an intent of the user from the internal concepts and the entities (block 1910) is performed by a critical thinking engine configured for this purpose. In some embodiments, the critical thinking engine is the critical thinking engine 108 of FIG. 1. In some embodiments, inferring an intent of the user from the internal concepts and the entities (block 1910) is accomplished as in Step 2 as earlier discussed in the context of “Analyzing Conversational Context As Part of Conversational Analysis”.

The method 1900 includes generating a logical framework 2000 for interpreting of the question (block 1912). In some embodiments, generating a logical framework 2000 for interpreting of the question (block 1912) is performed by a critical thinking engine configured for this purpose. In some embodiments, the critical thinking engine is the critical thinking engine 108 of FIG. 1. In some embodiments, generating a logical framework 2000 for interpreting of the question (block 1912) is accomplished as in Step 5 as earlier discussed in the context of “Analyzing Conversational Context As Part of Conversational Analysis”.

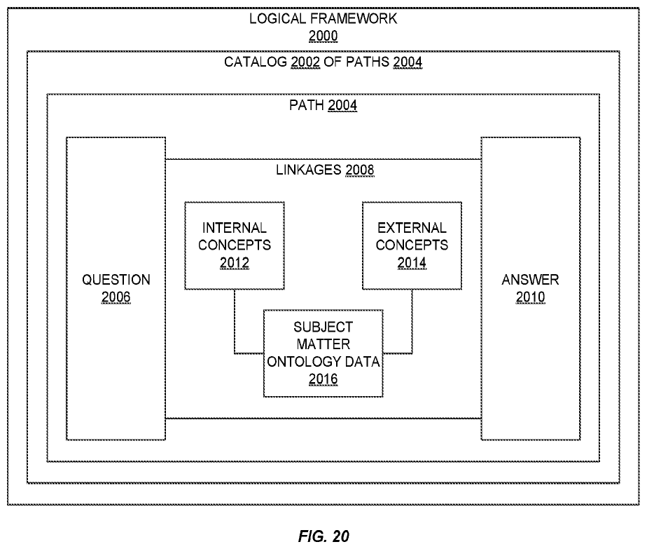

FIG. 20 shows an example logical framework 2000. The logical framework 2000 includes a catalog 2002 of paths 2004 from the question 2006 to respective answers 2010.

Each of the paths 2004 can include one or more linkages 2008 from the question 2006 to a path-specific answer 2010.

The linkages 2008 can include the internal concepts 2012 and external concepts 2014 derived from a store of subject matter ontology data 2016. In some embodiments, the store of subject matter ontology data 2016 is contained in a knowledge cloud. In some embodiments, the knowledge cloud is the knowledge cloud 102 of FIGS. 1 and 2. In some embodiments, the subject matter ontology data 2016 is the subject matter ontology data 216 of FIG. 2. In some embodiments, the subject matter ontology data 2016 includes the subject matter ontology 300 of FIG. 3.

The method 1900 shown in FIG. 19 further includes selecting a likely path from among the paths 2004 to a likely path-dependent answer based at least in part upon the intent of the user (block 1914). In some embodiments, selecting a likely path from among the paths 2004 to a likely path-dependent answer based at least in part upon the intent of the user (block 1914 is performed by a critical thinking engine configured for this purpose. In some embodiments, the critical thinking engine is the critical thinking engine 108 of FIG. 1. The selection can also be based in part upon the sufficiency of data to complete the linkages 2008. In some embodiments, selection can also be based in part upon the sufficiency of data to complete the linkages 2008 can be performed by a critical thinking engine that is further configured for this purpose. In some embodiments, the critical thinking engine is the critical thinking engine 108 of FIG. 1. The data can include user-specific data. The selection can also be based in part upon treatment sub-intents including tactical constituents related to the intent of the user by the store of subject matter ontology data 2016. In some embodiments, selection based in part upon treatment sub-intents including tactical constituents related to the intent of the user by the store of subject matter ontology data 2016 can be performed by a critical thinking engine further configured for this purpose. In some embodiments, the critical thinking engine is the critical thinking engine 108 of FIG. 1. The selection can further occur after requesting additional data from the user. An example of requesting additional data from the user is shown in FIG. 4 on line 406 “I need some additional information in order to answer this question, was this an in-home glucose test or was it done by a lab or testing service”. In some embodiments, the process of selection after requesting additional data from the user can be performed by a critical thinking engine further configured for this purpose. In some embodiments, the critical thinking engine is the critical thinking engine 108 of FIG. 1. In some embodiments, selecting a likely path from among the paths 2004 to a likely path-dependent answer based at least in part upon the intent of the user (block 1914) is accomplished through one or more of Steps 5-6 as earlier discussed in the context of “Analyzing Conversational Context As Part of Conversational Analysis”.

The method 1900 involves answering the question by following the likely path to the likely path-dependent answer (block 1916). In some embodiments, answering the question by following the likely path to the likely path-dependent answer (block 1916) is performed by a critical thinking engine configured for this purpose. In some embodiments, the critical thinking engine is the critical thinking engine 108 of FIG. 1. In some embodiments, answering the question by following the likely path to the likely path-dependent answer (block 1916) is accomplished as in Step 7 as earlier discussed in the context of “Analyzing Conversational Context As Part of Conversational Analysis”.

The method 1900 can further include relating inference groups of the internal concepts. In some embodiments, relating inference groups of the internal concepts is performed by a critical thinking engine further configured for this purpose. In some embodiments, the critical thinking engine is the critical thinking engine 108 of FIG. 1. Relating inference groups of the internal concepts can be based at least in part on shared entities for which each internal concept of an inference group of internal concepts describes a respective data attribute. In some embodiments, relating inference groups of the internal concepts based at least in part on shared entities for which each internal concept of an inference group of internal concepts describes a respective data attribute can be performed by a critical thinking engine further configured for this purpose. In some embodiments, the critical thinking engine is the critical thinking engine 108 of FIG. 1.

In some embodiments, the method 1900 of FIG. 19 is implemented as a computer program product in a computer-readable medium.

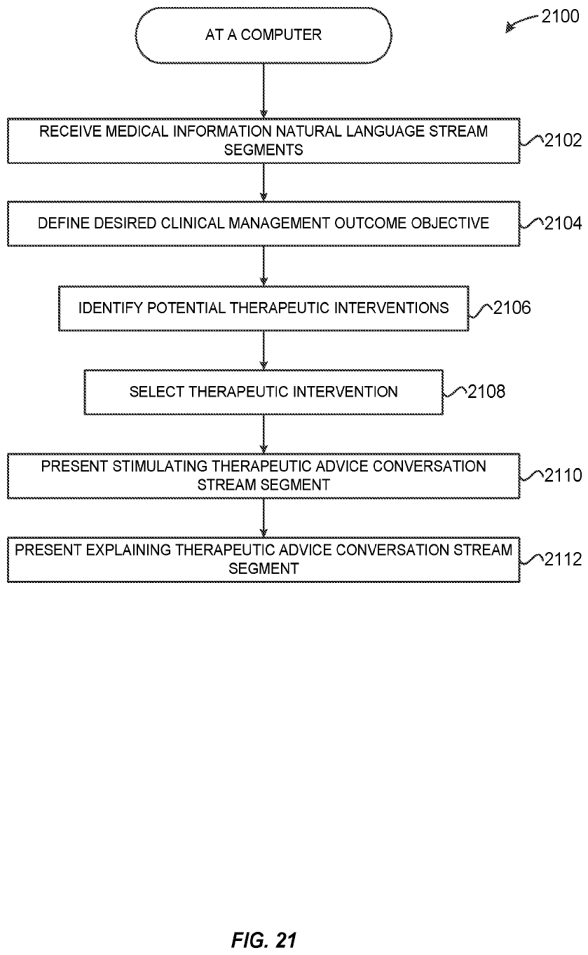

FIG. 21 shows a computer-implemented method 2100 for providing therapeutic medical action recommendations in response to a medical information natural language conversation stream. In some embodiments, the method 2100 is implemented as a computer program product in a non-transitory computer-readable medium. In some embodiments, the method 2100 of FIG. 21 is implemented as a system for providing therapeutic medical action recommendations in response to a medical information natural language conversation stream. The system can include a knowledge cloud, a critical thinking engine, and a cognitive agent. In some embodiments, the knowledge cloud is the knowledge cloud 102 of FIGS. 1 and 2. In some embodiments, the critical thinking engine is the critical thinking engine 108 of FIG. 1. In some embodiments, the cognitive agent is the cognitive agent 110 of FIG. 1.

In some embodiments, the method 2100 involves receiving segments of a medical information natural language conversation stream at an artificial intelligence-based health information conversation agent from a medical information conversation user interface (block 2102). In some embodiments the user interface is on the mobile device 104 of FIG. 1. In some embodiments, receiving segments of a medical information natural language conversation stream at an artificial intelligence-based health information conversation agent from a medical information conversation user interface (block 2102) is performed on a processor of a computer. In some embodiments, receiving segments of a medical information natural language conversation stream at an artificial intelligence-based health information conversation agent from a medical information conversation user interface (block 2102) is performed at a knowledge clout configured for this purpose. In some embodiments, receiving segments of a medical information natural language conversation stream at an artificial intelligence-based health information conversation agent from a medical information conversation user interface (block 2102) is Step 1 as earlier discussed in the context of “Analyzing Conversational Context As Part of Conversational Analysis”.

In some embodiments, the method 2100 further involves defining a desired clinical management outcome objective relevant to health management criteria and related health management data attributes of the user medical information profile in response to medical information content of a user medical information profile associated with the medical information natural language conversation stream (block 2104). In some embodiments, defining a desired clinical management outcome objective relevant to health management criteria and related health management data attributes of the user medical information profile in response to medical information content of a user medical information profile associated with the medical information natural language conversation stream (block 2104) is performed on a processor of a computer. In some embodiments, defining a desired clinical management outcome objective relevant to health management criteria and related health management data attributes of the user medical information profile in response to medical information content of a user medical information profile associated with the medical information natural language conversation stream (block 2104) is performed by a critical thinking engine configured for this purpose.

In some embodiments, defining a desired clinical management outcome objective relevant to health management criteria and related health management data attributes of the user medical information profile in response to medical information content of a user medical information profile associated with the medical information natural language conversation stream (block 2104) is accomplished through one or more of Steps 2-6 as earlier discussed in the context of “Analyzing Conversational Context As Part of Conversational Analysis”.

In some embodiments, the method 2100 further involves identifying a set of potential therapeutic interventions correlated to advancement of the clinical management outcome objective (block 2106). In some embodiments, identifying a set of potential therapeutic interventions correlated to advancement of the clinical management outcome objective (block 2106) is performed on a processor of a computer. In some embodiments, identifying a set of potential therapeutic interventions correlated to advancement of the clinical management outcome objective (block 2106) is performed by a critical thinking engine configured for this purpose. In some embodiments, identifying a set of potential therapeutic interventions correlated to advancement of the clinical management outcome objective (block 2106) is accomplished through one or more of Steps 2-6 as earlier discussed in the context of “Analyzing Conversational Context As Part of Conversational Analysis”.

In some embodiments, the method 2100 further involves selecting from among the set of potential therapeutic interventions correlated to advancement of the clinical management outcome objective a medical intervention likely to advance the clinical management outcome objective (block 2108). In some embodiments, selecting from among the set of potential therapeutic interventions correlated to advancement of the clinical management outcome objective a medical intervention likely to advance the clinical management outcome objective (block 2108) is based on a set of factors including the likelihood of patient compliance with the a recommendation for the a medical intervention and a statistical likelihood that the action will materially advance the clinical management outcome objective. In some embodiments, selecting from among the set of potential therapeutic interventions correlated to advancement of the clinical management outcome objective a medical intervention likely to advance the clinical management outcome objective (block 2108) is based on a set of factors comprising likelihood total expected cost expectation associated with the recommendation for the a medical intervention likely to advance the clinical management outcome objective. In some embodiments, selecting from among the set of potential therapeutic interventions correlated to advancement of the clinical management outcome objective a medical intervention likely to advance the clinical management outcome objective (block 2108) is performed on a processor of a computer. In some embodiments, selecting from among the set of potential therapeutic interventions correlated to advancement of the clinical management outcome objective a medical intervention likely to advance the clinical management outcome objective (block 2108) is performed by a critical thinking engine configured for this purpose. In some embodiments, selecting from among the set of potential therapeutic interventions correlated to advancement of the clinical management outcome objective a medical intervention likely to advance the clinical management outcome objective (block 2108) is accomplished through one or more of Steps 2-6 as earlier discussed in the context of “Analyzing Conversational Context As Part of Conversational Analysis”.

In some embodiments, the method 2100 further involves presenting in the medical information natural language conversation stream a therapeutic advice conversation stream segment designed to stimulate execution of the medical intervention likely to advance the clinical management outcome objective (block 2110). In some embodiments, the stimulation can be a motivation. In some embodiments, presenting in the medical information natural language conversation stream a therapeutic advice conversation stream segment designed to stimulate execution of the medical intervention likely to advance the clinical management outcome objective (block 2110) includes presenting to the user in the medical information natural language conversation stream a therapeutic advice conversation stream segment explaining a cost-benefit analysis comparing likely results of performance of the action likely to advance the clinical management outcome objective and likely results of non-performance of the action likely to advance the clinical management outcome objective. In some embodiments, presenting in the medical information natural language conversation stream a therapeutic advice conversation stream segment designed to stimulate execution of the medical intervention likely to advance the clinical management outcome objective (block 2110) includes presenting to the user in the medical information natural language conversation stream a conversation stream reinforcing the recommendation after expiration of a delay period. In some embodiments, presenting in the medical information natural language conversation stream a therapeutic advice conversation stream segment designed to stimulate execution of the medical intervention likely to advance the clinical management outcome objective (block 2110) includes presenting to the user in the medical information natural language conversation stream a therapeutic advice conversation stream segment explaining reasons for selection of the clinical management outcome objective. In some embodiments, presenting in the medical information natural language conversation stream a therapeutic advice conversation stream segment designed to stimulate execution of the medical intervention likely to advance the clinical management outcome objective (block 2110) includes notifying third party service providers of the clinical management outcome objective and the recommendation. In some embodiments, presenting in the medical information natural language conversation stream a therapeutic advice conversation stream segment designed to stimulate execution of the medical intervention likely to advance the clinical management outcome objective (block 2110) is performed on a processor of a computer. In some embodiments, presenting in the medical information natural language conversation stream a therapeutic advice conversation stream segment designed to stimulate execution of the medical intervention likely to advance the clinical management outcome objective (block 2110) is performed by a cognitive agent configured for this purpose. In some embodiments, presenting in the medical information natural language conversation stream a therapeutic advice conversation stream segment designed to stimulate execution of the medical intervention likely to advance the clinical management outcome objective (block 2110) is Steps 7 as earlier discussed in the context of “Analyzing Conversational Context As Part of Conversational Analysis”.

In some embodiments, the method 2100 further involves presenting to the user in the medical information natural language conversation stream a therapeutic advice conversation stream segment explaining a correlation between the medical intervention likely to advance the clinical management outcome objective and achievement of the clinical management outcome objective (block 2112). In some embodiments, presenting to the user in the medical information natural language conversation stream a therapeutic advice conversation stream segment explaining a correlation between the medical intervention likely to advance the clinical management outcome objective and achievement of the clinical management outcome objective (block 2112) is performed on a processor of a computer. In some embodiments, presenting to the user in the medical information natural language conversation stream a therapeutic advice conversation stream segment explaining a correlation between the medical intervention likely to advance the clinical management outcome objective and achievement of the clinical management outcome objective (block 2112) is performed by a critical thinking engine configured for this purpose. In some embodiments, presenting to the user in the medical information natural language conversation stream a therapeutic advice conversation stream segment explaining a correlation between the medical intervention likely to advance the clinical management outcome objective and achievement of the clinical management outcome objective (block 2112) is Steps 7 as earlier discussed in the context of “Analyzing Conversational Context As Part of Conversational Analysis”.

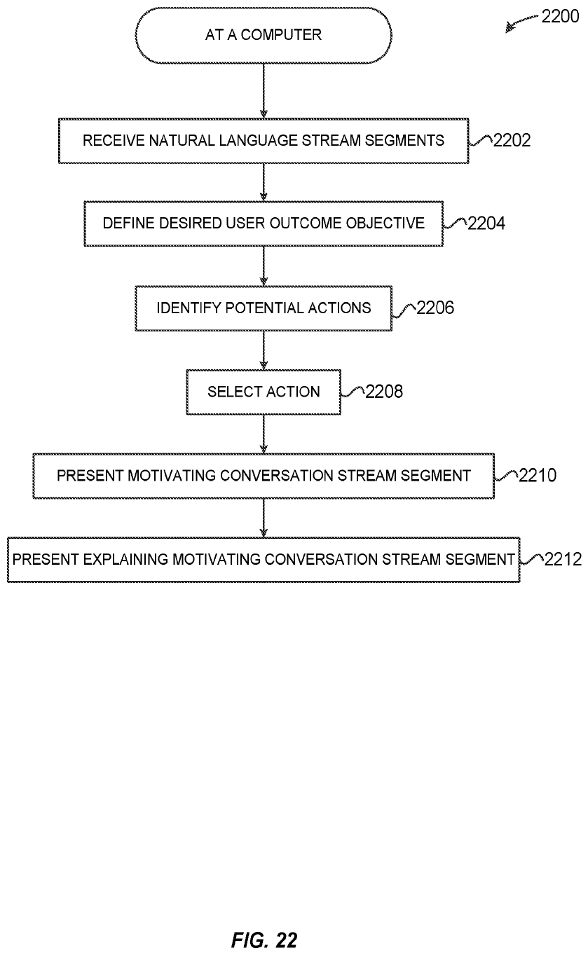

FIG. 22 shows a computer-implemented method 2200 for providing action recommendations in response to a natural language conversation stream. In some embodiments, the method 2200 is implemented as a computer program product in a non-transitory computer-readable medium. In some embodiments, the method 2200 of FIG. 22 is implemented as a system for providing action recommendations in response to a natural language conversation stream. The system can include a knowledge cloud, a critical thinking engine, and a cognitive agent. In some embodiments, the knowledge cloud is the knowledge cloud 102 of FIGS. 1 and 2. In some embodiments, the critical thinking engine is the critical thinking engine 108 of FIG. 1. In some embodiments, the cognitive agent is the cognitive agent 110 of FIG. 1.

In some embodiments, the method 2200 involves receiving segments of a natural language conversation stream at an artificial intelligence-based health information conversation agent from a conversation user interface (block 2202). In some embodiments the user interface is on the mobile device 104 of FIG. 1. In some embodiments, receiving segments of a natural language conversation stream at an artificial intelligence-based health information conversation agent from a conversation user interface (block 2202) is performed on a processor of a computer. In some embodiments, receiving segments of a natural language conversation stream at an artificial intelligence-based health information conversation agent from a conversation user interface (block 2202) is performed at a knowledge clout configured for this purpose. In some embodiments, receiving segments of a natural language conversation stream at an artificial intelligence-based health information conversation agent from a conversation user interface (block 2202) is Step 1 as earlier discussed in the context of “Analyzing Conversational Context As Part of Conversational Analysis”.

In some embodiments, the method 2200 further involves defining a desired user outcome objective relevant to health management criteria and related health management data attributes of the user profile in response to content of a user profile associated with the natural language conversation stream (block 2204). In some embodiments, defining a desired user outcome objective relevant to health management criteria and related health management data attributes of the user profile in response to content of a user profile associated with the natural language conversation stream (block 2204) is performed on a processor of a computer. In some embodiments, defining a desired user outcome objective relevant to health management criteria and related health management data attributes of the user profile in response to content of a user profile associated with the natural language conversation stream (block 2204) is performed by a critical thinking engine configured for this purpose.

In some embodiments, defining a desired user outcome objective relevant to health management criteria and related health management data attributes of the user profile in response to content of a user profile associated with the natural language conversation stream (block 2204) is accomplished through one or more of Steps 2-6 as earlier discussed in the context of “Analyzing Conversational Context As Part of Conversational Analysis”.

In some embodiments, the method 2200 further involves identifying a set of potential actions correlated to advancement of the user outcome objective (block 2206). In some embodiments, identifying a set of potential actions correlated to advancement of the user outcome objective (block 2206) is performed on a processor of a computer. In some embodiments, identifying a set of potential actions correlated to advancement of the user outcome objective (block 2206) is performed by a critical thinking engine configured for this purpose. In some embodiments, identifying a set of potential actions correlated to advancement of the user outcome objective (block 2206) is accomplished through one or more of Steps 2-6 as earlier discussed in the context of “Analyzing Conversational Context As Part of Conversational Analysis”.

In some embodiments, the method 2200 further involves selecting from among the set of potential actions correlated to advancement of the user outcome objective an action likely to advance the user outcome objective (block 2208). In some embodiments, selecting from among the set of potential actions correlated to advancement of the user outcome objective an action likely to advance the user outcome objective (block 2208) is based on a set of factors including the likelihood of patient compliance with the a recommendation for the an action and a statistical likelihood that the action will materially advance the user outcome objective. In some embodiments, selecting from among the set of potential actions correlated to advancement of the user outcome objective an action likely to advance the user outcome objective (block 2208) is based on a set of factors comprising likelihood total expected cost expectation associated with the recommendation for the an action likely to advance the user outcome objective. In some embodiments, selecting from among the set of potential actions correlated to advancement of the user outcome objective an action likely to advance the user outcome objective (block 2208) is performed on a processor of a computer. In some embodiments, selecting from among the set of potential actions correlated to advancement of the user outcome objective an action likely to advance the user outcome objective (block 2208) is performed by a critical thinking engine configured for this purpose. In some embodiments, selecting from among the set of potential actions correlated to advancement of the user outcome objective an action likely to advance the user outcome objective (block 2208) is accomplished through one or more of Steps 2-6 as earlier discussed in the context of “Analyzing Conversational Context As Part of Conversational Analysis”.

In some embodiments, the method 2200 further involves presenting in the natural language conversation stream a conversation stream segment designed to motivate performance of the action likely to advance the user outcome objective (block 2210). In some embodiments, presenting in the natural language conversation stream a conversation stream segment designed to motivate performance of the action likely to advance the user outcome objective (block 2210) includes presenting to the user in the natural language conversation stream a conversation stream segment explaining a cost-benefit analysis comparing likely results of performance of the action likely to advance the user outcome objective and likely results of non-performance of the action likely to advance the user outcome objective. In some embodiments, presenting in the natural language conversation stream a conversation stream segment designed to motivate performance of the action likely to advance the user outcome objective (block 2210) includes presenting to the user in the natural language conversation stream a conversation stream reinforcing the recommendation after expiration of a delay period. In some embodiments, presenting in the natural language conversation stream a conversation stream segment designed to motivate performance of the action likely to advance the user outcome objective (block 2210) includes presenting to the user in the natural language conversation stream a conversation stream segment explaining reasons for selection of the user outcome objective. In some embodiments, presenting in the natural language conversation stream a conversation stream segment designed to motivate performance of the action likely to advance the user outcome objective (block 2210) includes notifying third party service providers of the user outcome objective and the recommendation. In some embodiments, presenting in the natural language conversation stream a conversation stream segment designed to motivate performance of the action likely to advance the user outcome objective (block 2210) is performed on a processor of a computer. In some embodiments, presenting in the natural language conversation stream a conversation stream segment designed to motivate performance of the action likely to advance the user outcome objective (block 2210) is performed by a cognitive agent configured for this purpose. In some embodiments, presenting in the natural language conversation stream a conversation stream segment designed to motivate performance of the action likely to advance the user outcome objective (block 2210) is Steps 7 as earlier discussed in the context of “Analyzing Conversational Context As Part of Conversational Analysis”.

In some embodiments, the method 2200 further involves presenting to the user in the natural language conversation stream a conversation stream segment explaining a correlation between the action likely to advance the user outcome objective and achievement of the user outcome objective (block 2212). In some embodiments, presenting to the user in the natural language conversation stream a conversation stream segment explaining a correlation between the action likely to advance the user outcome objective and achievement of the user outcome objective (block 2212) is performed on a processor of a computer. In some embodiments, presenting to the user in the natural language conversation stream a conversation stream segment explaining a correlation between the action likely to advance the user outcome objective and achievement of the user outcome objective (block 2212) is performed by a critical thinking engine configured for this purpose. In some embodiments, presenting to the user in the natural language conversation stream a conversation stream segment explaining a correlation between the action likely to advance the user outcome objective and achievement of the user outcome objective (block 2212) is Steps 7 as earlier discussed in the context of “Analyzing Conversational Context As Part of Conversational Analysis”.

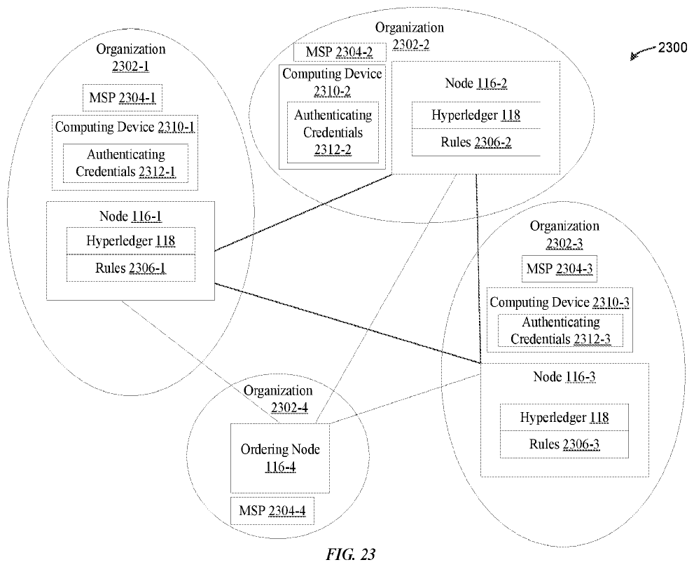

FIG. 23 shows distributed hyperledger fabric network 2300 of nodes 116 each maintaining a copy of a hyperledger 118 to manage knowledge in a healthcare ecosystem, in accordance with various embodiments. The distributed hyperledger fabric network 2300 is divided into different organizations 2302 that may include one or more nodes 116 associated with that particular organization. An organization 2302 may refer to a security domain, unit of identity, and/or authenticating credentials. Each organization 2302 may include one or more nodes 116 that are associated with a particular entity. For example, one organization 2302-1 may be associated with one or more patient entities that are registered as one or more nodes 116-1, another organization 2302-2 may be associated with one or more medical personnel entities that are registered as one or more nodes 116-2, another organization 2302-3 may be associated with one or more medical facility entities that are registered as one or more nodes 116-3, and so on for any suitable entities (e.g., insurance providers, government agencies, professional associations, etc.) in a healthcare ecosystem.

In some embodiments, the organizations 2302 may be associated with a combination of entities. For example, the entities that are associated with a hospital may be grouped into an organization 2302. The organization 2302 may be associated with a medical facility entity for the hospital, one or more medical personnel entities for the physicians, nurses, staff, etc., and/or one or more patient entities for each patient of the hospital.

Other organizations 2302 may be associated with nodes 116 representing services provided by the distributed hyperledger fabric network 2300. For example, organization 2302-4 is associated with an ordering node 116-4 that ensures that the one or more rules implemented by each of the nodes 116-1, 116-2, and/or 116-3 involved in a transaction are satisfied and/or there is consensus among the nodes 116-1, 116-2, and/or 116-3 prior to approving performance of the transaction and addition of the transaction into the hyperledger 118. Using the ordering node 116-4 enhances consistency and security of the hyperledger 118 by controlling what is allowed to be added to the hyperledger 118.

Each node 116 may implement various rules 2306 which may be installed into the hyperledger 118. In some embodiments, the rules 2306 may be included in each respective copy of the hyperledger 118 that is distributed between the various nodes 116. In some embodiments, a hyperledger 118 on one node (e.g., 116-1) may have a first subset of the rules 2306 installed and another node (e.g., 116-2) may have a second subset of the rules 2306 installed in its copy of the hyperledger 118 where at least one rule in the first subset is different than a rule in the second subset. The rules 2306 may be implemented as computer executable instructions (e.g., software modules).

The rules 2306 may be self-executing at certain frequencies. For example, after a period of time expires, a rule 2306 may determine whether certain information (e.g., authorizing credential) of an entity (e.g., medical personnel entity) registered as a node 116 needs to be updated in the hyperledger 118 and provide a notification to a computing device 2310 used by that entity. In another example, a rule 2306 may determine that content that is stored on the hyperledger 118 needs to be verified and/or updated after a monitored period of time expires since the content was last verified, updated, or generated. In other embodiments, the rules 2306 may be self-executing based on certain conditions occurring. For example, when an authorizing credential of an entity expires in the hyperledger 118, the rule 2306 may trigger a notification to be sent to the computing device 2310 of that entity. In other instances, the rules 2306-2 may be triggered when a request to perform a transaction on the hyperledger 118 is received.

In general, the rules 2306 may be used to govern the content allowed to be stored on the hyperledger 118, to control who is permitted to store content on the hyperledger 118, to control who is permitted to update content on the hyperledger 118, to control who is permitted to verify the source of the content, and/or to control who has access to the content, among other things. The rules 2306 may specify when updates to the hyperledger 118 are to be provided. The rules 2306 may be analytics-based in that they monitor states or conditions of information in the hyperledger 118, the computing devices 2310, and/or the nodes 116, and determine when hyperledger 118 updates should be provided. For example, the rules 2306 may specify that updates to the hyperledger 118 are to be provided based on any combination of geofencing (e.g., geolocation of the computing devices 2310 associated with particular nodes 116), authorizing credentials of medical personnel being valid, time frames for verifying content, and so forth.

Each organization 2302 may include a computing device 2310 used by an entity associated with that organization 2302. The computing device 2310 may include one or more memories, processors, and/or network interfaces. The computing device 2310 may be similar to any computing device described with respect to FIG. 14. The user may use the computing device 2310 to send requests to perform transactions using the hyperledger 118 to the cognitive intelligence platform 102 that may include the nodes 116.

Each organization 2302 may include a membership service provider (MSP) 2304 that is responsible for issuing identities and authenticating credentials 2312 to computing devices 2310 associated with entities. As described herein, when a computing device 2308 requests to perform a transaction, such as registering as a node 116 on the distributed hyperledger fabric network 2300, the computing device 2310 may provide certain information pertaining to the entity. For example, for a medical personnel entity the information may include at least an identity of the medical personnel entity, an authorizing credential (e.g., NPI number, medical license number, etc.), a date the authorizing credential was last updated, an address of a place of work of the medical personnel entity, gender, race, and so forth. For a patient entity, the information may include at least the patient's identity, social security number, driver's license number, address, medical records, allergies, medicine allergies, familial medical history, and so forth. The information may be encrypted and securely stored on the hyperledger 118 such that only appropriate entities can view the information of another entity.

The nodes 116 may communicate with each other to determine if a consensus is reached as to whether to allow the transaction to be performed. Further, one or more of the rules 2306 may be applied to determine whether to allow the transaction to be performed. When the consensus protocol and/or the rules 2306 are satisfied, the ordering node 116 may order the transaction to be performed and a record of the transaction is added to the hyperledger 118.

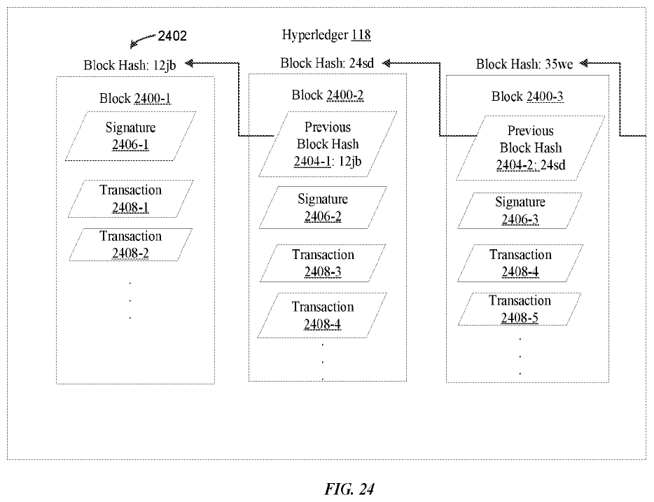

FIG. 24 shows an example hyperledger 118, in accordance with various embodiments. As depicted, the hyperledger 118 includes three blocks 2400-1, 2400-2, and 2400-3. Each of the blocks is cryptographically linked to a previous block. Each block 2400 includes a block hash 2402 for that block that is determined using a suitable hash function. For example, block 2400-1 has block hash 2402 “12jb”, block 2400-2 has block hash 2402 “24sd”, and block 2400-3 has block hash 2402 “35we”. The blocks 2400 are cryptographically linked together by including a block header with the block hash of the previous block. For example, block 2400-2 includes a block header including previous block hash 2404-1 with value “12jb”, which is the value of the previous block 2400-1 in the hyperledger 118, and block 2400-3 includes a block header including previous block hash 2404-2 with value “24sd”, which is the value of the previous block 2400-2.

Each block 2400 includes a signature 2406 and one or more transactions 2408. The signature 2406 may be the identity of the entity that requested the transaction to be performed. If there are multiple transactions 2408 stored on a block 2400 and different entities are associated with those transactions 2408, there may be different signatures 2406 associated with the different entities stored on the blocks 2400. The signatures may be used to prove that the entity is the source for a transaction 2408 (e.g., generated and stored certain content). In some embodiments, a block 2400 storing transactions 2408 may be added to the hyperledger 118 after it is determined that the one or more rules 2306 and/or consensus between the nodes 116 are satisfied. The transactions 2408 in a given request may be grouped and added as a block 2400 to the hyperledger 118, or different transactions from different requests may be grouped and added as a block 2400 if the transactions are related or involve particular nodes 116.

In some embodiments, the transactions 2408 relating to registering an entity may store identifying information pertaining to an entity, such as an identity, address, social security number, driver's license number, and so forth. These transactions 2408 may store authorizing credentials, such as license numbers (NPIs) for physicians, license numbers for pharmacists, license numbers for pharmacies to dispense medicine, and so forth.

The transactions 2408 relating to storing content on the hyperledger 118 may store text, videos, images, etc. generated by an entity. For example, the content may relate to healthcare and include evidence-based guidelines, knowledge representation, clinical studies, clinical processes, clinical techniques, treatment plans, and so forth. The transactions 2408 may store various information for each content that is added to the hyperledger 118. The information may include a title, a document type, an author, an authorizing credential (e.g., medical license number, medical degree, etc.) of the author, an upload timestamp, a last update timestamp, content, an indicator of whether the content has been verified by one or more other licensed professionals, an identity of the one or more other licensed professionals, one or more authorizing credentials of the one or more other licensed professionals, and so forth.

The transactions 2408 relating to providing content on the hyperledger 118 may store an incremented counter value for a number of views of the content, a timestamp of the viewing of the content, as well as any information pertaining to the requesting entity. For example, the hyperledger 118 may determine whether the requesting entity is associated with an authorized credential and increment a counter value that indicates the content was viewed by another entity having an authorized credential and store the timestamp that the content was provided to a computing device associated with the requesting entity. Other counters may be used, such as a counter for how many medical personnel entities having valid authorized credentials verified the content is accurate and good medical practice. Content associated with a higher number of views and/or verifications by entities having authorizing credentials may be more trustworthy and selected to be provided by the rules when a user requests content.

In this regard, a ranking technique may be used by the rules to identify the content that is associated with a highest number of total views by the entities (e.g., patients, medical personnel), a highest number of total views by medical personnel entities having valid authorizing credentials, a highest number of total verifications by medical personnel entities having valid authorizing credentials, and so forth. Providing the content that is viewed by more medical personnel over time than other content may enable the user to obtain more trustworthy content faster. As a result, processing, memory, and/or network resources may be saved by providing more trustworthy content using the hyperledger 118 because the user may not perform multiple searches to find other content if initially provided with the most trustworthy content.

The transactions 2408 relating to storing updated content on the hyperledger 118 may store the updated content that may include additional content in conjunction with original content. The additional content may include text documents, videos, images, etc. generated by an entity. The entity may be the same entity that generated the original content or may be a different entity than the entity that stored the original content. The updated content may relate to healthcare and include evidence-based guidelines, knowledge representation, clinical studies, clinical processes, clinical techniques, treatment plans, and so forth. The rules 2306 may ensure that least a portion of the additional content is new relative to other content stored in the hyperledger 118. The transactions 2408 may store various information for each updated content that is added to the hyperledger 118. The information may include a title, a document type, an author, an authorizing credential (e.g., medical license number, medical degree, etc.) of the author, an upload timestamp, a last update timestamp, updated content (e.g., original content and additional content), an indicator of whether the content has been verified by one or more other licensed professionals, an identity of the one or more other licensed professionals, one or more authorizing credentials of the one or more other licensed professionals, and so forth.

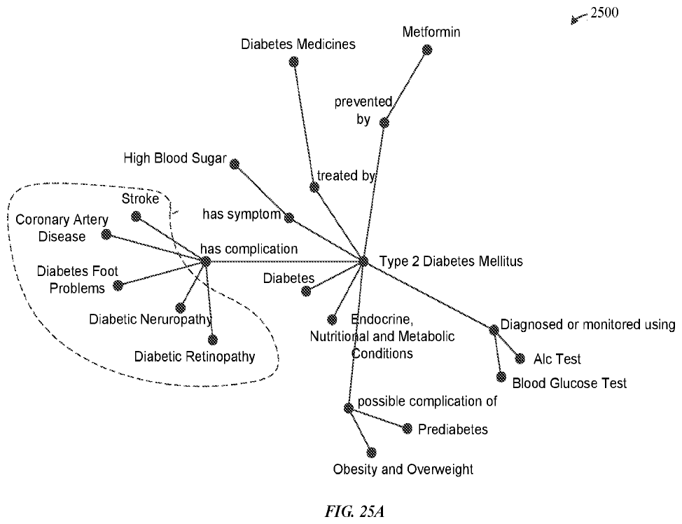

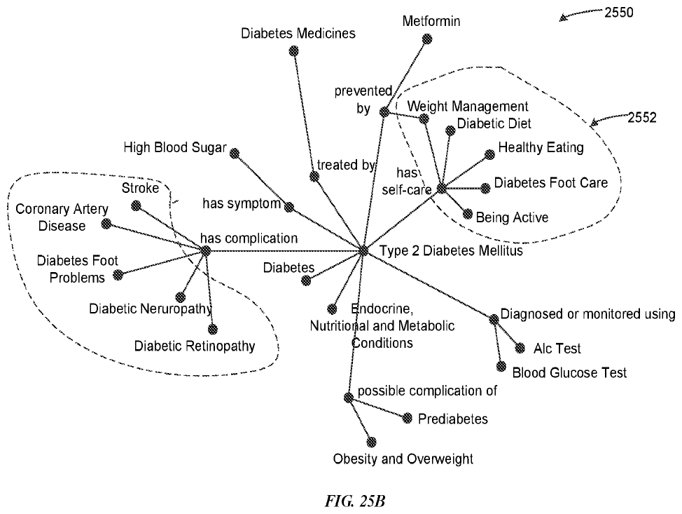

FIG. 25A shows a cognitive map or “knowledge graph” 2500 at a first knowledge stage, in accordance with various embodiments. The knowledge graph 2500 may represent a representation of knowledge pertaining to Type 2 diabetes at a first knowledge stage including original content. The original content may include nodes in the knowledge graph 2500 that represent a health artifact or relationship for a particular patient of a medical personnel entity. The nodes may be generated from direct interrogation or indirect interactions with the user (by way of the user device 104). The original content including the nodes may include symptoms (High Blood Sugar), possible complications (Prediabetes, Obesity and Overweight), actual complications (e.g., Stroke, Coronary Artery Disease, Diabetes Foot Problems, Diabetic Neuropathy, Diabetic Retinopathy), how the medical condition is diagnosed or monitored (e.g., Alc Test, Blood Glucose Test), how the medical condition is treated (e.g., Diabetes Medicines), how the medical condition is prevented (e.g., Metformin), Diabetes, Endocrine, Nutritional, and Metabolic Conditions, and so forth.

A medical personnel entity may use a computing device 2310 to transmit a request to store the knowledge graph 2500 in the hyperledger 118. The hyperledger 118 may execute one or more rules to (i) determine that the user has proper authenticating credentials, (ii) determine that the user has valid authorizing credentials (e.g., medical degree from an accredited school, valid medical license, etc.), (iii) determine that the original content in the knowledge graph 2500 is not disclosed by other content stored in the hyperledger 118, and so forth. Further, the nodes may use a consensus protocol to determine whether to allow the transaction to be performed. If the one or more rules and/or the consensus protocol are satisfied, the knowledge graph 2500 may be stored in the hyperledger 118.

The medical personnel entity may input additional data that is represented in the knowledge graph 2550 in FIG. 25B. The knowledge graph 2550 evolved to a second knowledge stage based on the addition of additional content in the nodes in dotted area 2552 that pertain to a new self-care section for Type 2 Diabetes. The medical personnel entity may determine to add the new self-care section to the original content including the nodes previously described with reference to FIG. 25A. The self-care section may include nodes for Weight Management, Diabetic Diet, Healthy Eating, Diabetes Foot Care, and Being Active. Each node may include specific details pertaining to the respective topic that instruct the patient how to take care of themselves to reduce symptoms and/or overcome the medical condition represented in the knowledge graph 2550.

It should be noted that the additional content including the new self-care section may be unique for treating Type 2 Diabetes for a patient relative to other content that relates to treating Type 2 Diabetes. Accordingly, the medical personnel entity may use a computing device 2310 to transmit a transaction request to store the knowledge graph 2550 on the hyperledger 118. The hyperledger may execute the one or more rules to (i) determine that the user has proper authenticating credentials, (ii) determine that the user has valid authorizing credentials (e.g., medical degree from an accredited school, valid medical license, etc.), (iii) determine that at least a portion of the additional content in the knowledge graph 2550 is not disclosed by other content stored in the hyperledger 118, and so forth. Further, the nodes may use a consensus protocol to determine whether to allow the transaction to be performed. If the one or more rules and/or the consensus protocol are satisfied, the knowledge graph 2550 may be stored in the hyperledger 118. A timestamp may be stored with the updated content each time updated content is stored on the hyperledger 118 to show a time series evolution of the content since the original content was first stored on the hyperledger 118 to a current state of the updated content.

The new self-care section may prove to provide great results for treating Type 2 Diabetes. Other medical personnel entities that are associated with nodes in the distributed hyperledger fabric network 2300 may request access to the knowledge graph 2550 stored in the hyperledger 118 using computing devices 2310. If the knowledge graph 2550 is set to public, the hyperledger 118 may provide the knowledge graph 2550 to the requesting entities. If the knowledge graph 2550 is set to private, the hyperledger 188 may provide the knowledge graph 2550 upon the requesting entity purchasing an access right to the knowledge graph 2550, or in various other scenarios.

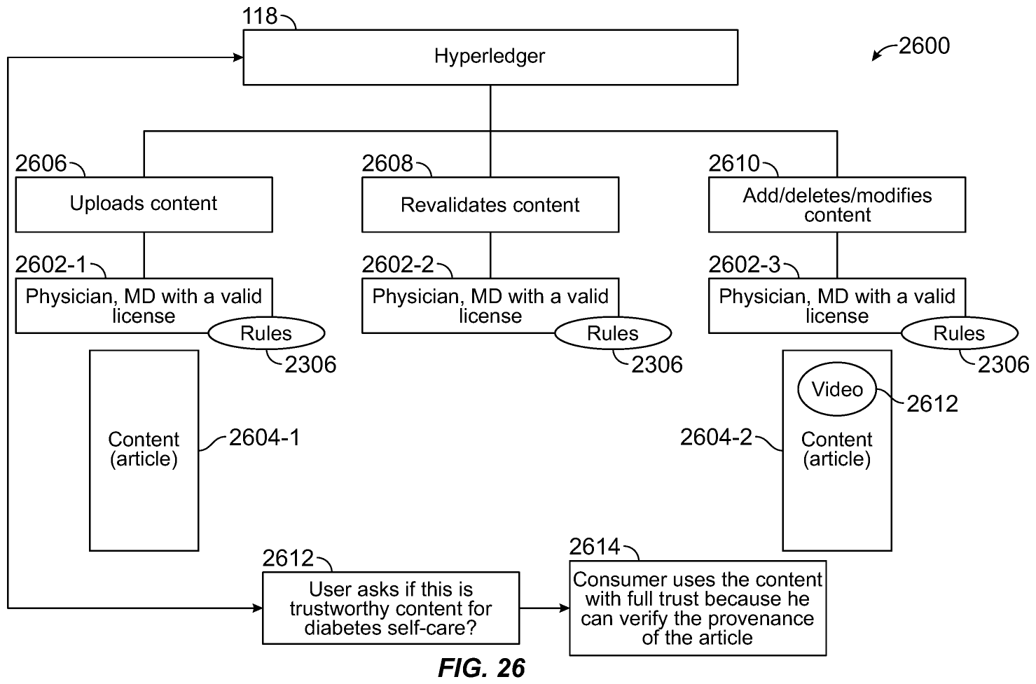

FIG. 26 shows a general process 2600 for performing transaction requests on a hyperledger 118 by various nodes 116 in a healthcare ecosystem, in accordance with various embodiments. One or more rules 2306 may be used to determine when to allow transaction requests to be performed on the hyperledger 118. The rules 2306 may be computer instructions executable by one or more processors of a node 116 (2602-1, 2602-2, 2602-3) representing an entity. The rules 2306 may be installed in the hyperledger 118 and may specify scenarios when updates to the hyperledger 118 based on analytics and/or when to allow transactions to be performed on the hyperledger 118.

In one scenario, the rules 2306 may specify that the authorizing credential of a node 2602-1 representing a medical personnel entity has to be updated every X period of time in the hyperledger 118. For example, a physician's 2502 medical license has to be updated every 3 years, and a pharmacist's 2504 license has to be updated every 5 years. The rules 2306 may analyze the information pertaining to the medical personnel 2602-1 in the transactions 2408 stored in the hyperledger 118 and may determine that the period of time for updating the authorizing credential has expired or is about to expire. As a result, the node executing the rules 2306 may cause a notification to be presented on the computing device 2310 used by the medical personnel entity that instructs the medical personnel entity to update their authorizing credential. The updated authorizing credentials may be stored on the hyperledger 118.

In one embodiment, the rules 2306 may specify that the hyperledger 118 is updated when a transaction, such as a request to upload (2606) content 2604-1, is received at a node 2602-1 associated with a physician, for example, and various checks made by the rules 2306 are satisfied and/or a consensus protocol used by the nodes 116 is satisfied. The content 2604-1 may be an article pertaining to healthcare that includes text and/or images. For example, the content 2604-1 may describe aspects of the knowledge graph 2500 for Type 2 Diabetes. The rules 2306 may determine (i) whether the physician requesting to store the content 2604-1 has valid authenticating credentials (e.g., username, password, unique identifier), (ii) whether the physician requesting to store the content 2604-1 has valid authorizing credentials (e.g., medical degree, medical license, etc.), and/or (iii) whether at least a portion of the content 2604-1 is new relative to other content in the hyperledger 118 pertaining to the same topic. If the rules 2306 are satisfied and/or a consensus protocol used by the nodes 116 is satisfied, the content 2604-1 may be stored on the hyperledger 118. In some embodiments, the consensus protocol may be satisfied when one or more of the rules 2306 are satisfied.

In one embodiment, the rules 2306 may specify that the content 2604-1 stored on the hyperledger 118 be updated every certain period of time by the physician that uploaded the content 2604-1 or by another physician with valid authorizing credentials. The rules 2306 may determine that a certain amount of time has elapsed since the content 2604-1 was generated, uploaded, and/or last verified, and may cause a notification to be transmitted to the computing device 2310 of an appropriate physician to verify the content 2604-1 is still valid.

Accordingly, at 2608, a computing device associated with the appropriate physician may transmit a request to review the content 2604-1 and verify the content 2604-1 is still valid medical practice/advice/treatment plan. The node 2602-2 may be associated with the same physician that uploaded the content 2604-1 or may be associated with another physician. The rules may determine (i) whether the physician requesting to verify the content 2604-1 has valid authenticating credentials (e.g., username, password, unique identifier), and/or (ii) whether the physician requesting to verify the content 2604-1 has valid authorizing credentials (e.g., medical degree, medical license, etc.). If the rules 2306 are satisfied and/or the consensus protocol is satisfied, the hyperledger 118 may provide the content 2604-1 to the computing device associated with the requesting physician and the requesting physician can review the content 2604-1 and provide an indication to the hyperledger, via the computing device, that the content 2604-1 is verified. In such an instance, the hyperledger 118 may update a last verified timestamp. If the physician reviews the content 2604-1 and determines that information in the content 2604-1 is outdated or no longer valid, the physician may provide, via the computing device, an indication that the content 2604-1 is not verified. The hyperledger 118 may prevent the content 2604-1 from being distributed upon further requests.

In one embodiment, the rules 2306 may specify that the hyperledger 118 is updated when a transaction, such as a request to add, delete, or modify (2610) content 2604-1, is received at a node 2602-3 associated with a physician, for example, and various checks made by the rules 2306 are satisfied and/or a consensus protocol used by the nodes 116 is satisfied. The node 2602-3 may be associated with the same physician that initially uploaded the content 2604-1 or may be associated with another physician.

When the request is to add content, the same description provided for 2606 above may apply. When the request is to delete content, the rules 2306 may determine (i) whether the physician requesting to delete the content 2604-1 has valid authenticating credentials (e.g., username, password, unique identifier), and/or (ii) whether the physician requesting to delete the content 2604-1 has valid authorizing credentials (e.g., medical degree, medical license, etc.). If the physician requesting deletion of the content 2604-1 is not the same physician that generated and uploaded the content 2604-1, then the consensus protocol may not be satisfied unless the node 2602-1 associated with the physician that uploaded the content 2604-1 agrees to allow the content 2604-1 to be deleted.

If the request is to modify content 2604-1 stored on the hyperledger 118, then the physician may have added additional content (e.g., a video 2612) to the original content to create updated content 2604-2. The additional content may explain different steps of a treatment plan. For example, the video 2612 in the updated content 2604-2 may present new steps of the self-care section for the knowledge graph 2550 in FIG. 25B. The rules 2306 may determine (i) whether the physician requesting to store the updated content 2604-2 has valid authenticating credentials (e.g., username, password, unique identifier), (ii) whether the physician requesting to store the updated content 2604-2 has valid authorizing credentials (e.g., medical degree, medical license, etc.), and/or (iii) whether at least a portion of the updated content 2604-2 is new relative to other content in the hyperledger 118 pertaining to the same topic. In this example, metadata of the video 2612 may be used, the audio in the video may be translated to text, and/or object character recognition may be used to determine whether the video 2612 includes new content. If the rules 2306 are satisfied and/or a consensus protocol used by the nodes 116 is satisfied, the updated content 2604-2 may be stored on the hyperledger 118. In some embodiments, the consensus protocol may be satisfied when one or more of the rules 2306 are satisfied.

In some embodiments, a user may be associated with a node 116 in the distributed hyperledger fabric network 2300. Accordingly, the user may provide authenticating credentials that are used with a software application executing on a computing device to access the cognitive intelligence platform 102 to submit transaction requests. In some embodiments, the user may obtain the content 2604-2. For example, the physician that generated the updated content 2604-2 may provide the updated content 2604-2 to the user, or the user may submit a transaction request to search the hyperledger 118 for content related to Type 2 Diabetes treatment. The hyperledger 118 may provide the updated content 2604-2 that includes the new self-care section. The user can query (2612) whether the updated content 2604-2 is trustworthy content for diabetes self-care. In some embodiments, the hyperledger 118 may provide the provenance of the content 2604-2 by presenting an identity of the physician that generated the updated content 2604-2, the authorizing credentials (e.g., medical degree, medical license, etc.) of the physician that generated the updated content 2604-2, a date the updated content 2604-2 was last verified, and so forth. Based on verification from the hyperledger 118, the user (consumer) may use (2614) the updated content 2604-2 with full trust because he can verify the provenance of the updated content 2604-2.

In some embodiments, the user may submit a question asking whether the content 2604-2 is trustworthy content in natural language to the cognitive intelligence platform 102. In some embodiments, the cognitive intelligence platform is the cognitive intelligence platform 102 as shown in FIG. 1. In some embodiments, the cognitive intelligence platform is implemented on the computing device 1400 shown in FIG. 14. The cognitive intelligence platform 102 may receive the question at the cognitive agent 110 and may process the question using the critical thinking engine 108, natural language database 122, knowledge cloud 106, and/or conversation orchestrator 124 using any of the methods disclosed herein. The cognitive intelligence platform 102 may determine, using the hyperledger 118, whether the content 2604-2 is trustworthy content by verifying that the content 2604-2 was generated by a medical personnel entity having a valid authorizing credential, and/or the content 2604-2 has been updated/verified within a certain time period. If the content 2604-2 is determined to be trustworthy, the cognitive intelligence platform 102 may cause an indication to be presented on the computing device of the user device 104. The indication may be text confirming the content 2604-2 is trustworthy and/or a visual representation (e.g., a thumbs up, check mark, etc.).

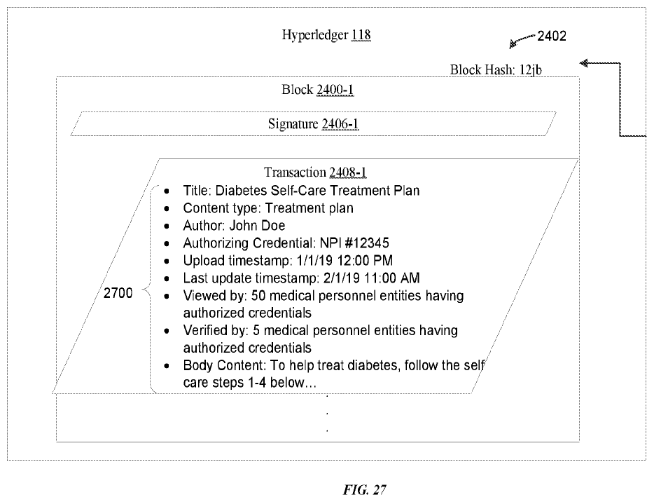

FIG. 27 shows example content 2700 stored on the hyperledger 118, in accordance with various embodiments. A medical personnel entity may have used a computing device 2310 to submit a transaction request to perform an operation on the hyperledger 118, where the operation included storing the content 2700 on the hyperledger 118. One or more rules 2306 may have determined that the requesting medical personnel entity has (i) valid authenticating credentials and/or (ii) valid authorizing credentials, and/or that the content 2700 includes at least a portion of Body Content that is new relative to other content stored in the hyperledger 118. In the depicted example, the one or more rules 2306 are satisfied, and the content 2700 is stored in the transaction 2408-1 in the block 2406-1 in the hyperledger 118. Further, the block 2400-1 includes a signature 2406-1 of the medical personnel entity that provided the content 2700. The signature 2406-1 may be a digital signature that uniquely identifies the medical personnel entity as the source of the content 2700.

In the depicted example, the content 2700 may describe the knowledge graph 2500 that included nodes representing original content related to Type 2 Diabetes. As depicted, the content 2700 may include various information, such as a Title (“Diabetes Self-Care Treatment Plan”), Content Type (“Treatment Plan”), Author (“John Doe”), Authorizing Credential (“NPI #12345”), Upload Timestamp (“Jan. 1, 2019 12:00 PM”), Last Updated Timestamp (“Feb. 1, 2019 11:00 AM”), Viewed By (“50 medical personnel entities having authorized credentials”), Verified By (“5 medical personnel entities having authorized credentials”), and/or Body Content (“To help treat diabetes, follow the self-care steps 1-4 below . . . ”).

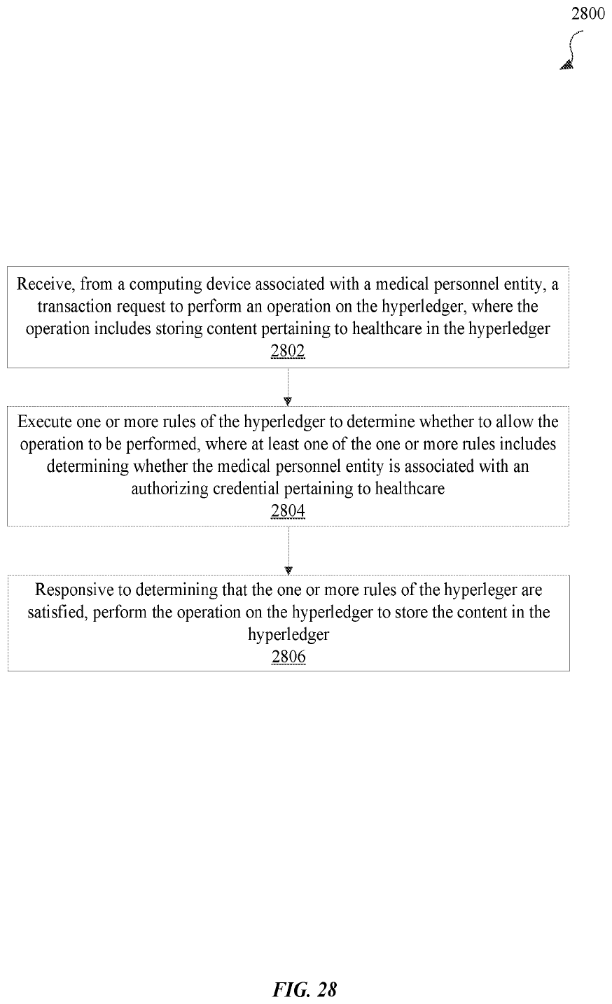

FIG. 28 shows a method 2800 for maintaining content pertaining to healthcare in a hyperledger 118, in accordance with various embodiments. In some embodiments, the method 2800 is implemented as a computer program product in a non-transitory computer-readable medium and executable by one or more processors of one or more computing devices described in the cognitive intelligence platform 102 of FIG. 1. In some embodiments, the method 2800 of FIG. 28 is implemented as a system for maintaining a hyperledger 118 for content at one or more nodes 116. The system can include components described in the cognitive intelligence platform 102.

In some embodiments, the method 2800 may involve receiving (2802), from a computing device 2310 associated with a medical personnel entity, a transaction request to perform an operation on the hyperledger 118, where the operation includes storing content 2700 pertaining to healthcare in the hyperledger 118. The content 2700 may be any suitable content, such as evidence-based guidelines, knowledge representation, description of a knowledge representation, clinical studies, clinical trial results, clinical process descriptions, treatment plans for medical conditions, and so forth. In some embodiments, the content 2700 includes a treatment plan where at least a portion of the treatment plan is written by the medical personnel entity. The hyperledger 118 may maintain other content (e.g., treatment plans) that are validated as being provided by other licensed medical doctors based on other authorizing credentials that are stored in the hyperledger 118.

The method 2800 may involve executing (2804) one or more rules 2306 of the hyperledger 118 to determine whether to allow the operation to be performed, where at least one of the one or more rules includes determining whether the medical personnel entity is associated with an authorizing credential (e.g., medical degree, NPI, medical license number) pertaining to healthcare. Further, the one or more rules may include validating the authorizing credential with a professional association or government agency that issued the authorizing credential. The one or more rules 2306 may search the hyperledger 118 for the authorizing credential for the medical personnel entity. The authorizing credential may be provided by the medical personnel entity in the transaction request and included in the content, and/or the authorizing credential may be stored at a transaction in the hyperledger 118. This particular rule 2306 may be satisfied when the authorizing credential of the medical personnel entity is valid. This particular rule 2306 may not be satisfied when the authorizing credential of the medical personnel entity is not valid. When the authorizing credential is not valid, the rule 2306 may prevent the content 2700 from being stored on the hyperledger 118.

The one or more rules 2306 may include determining, using one or more transactions stored in the hyperledger 118, whether at least a portion of the content 2700 is new relative to other content in the hyperledger 118. When the content 2700 includes text strings, the content 2700 may be parsed. The text strings may be tokenized into words, keywords, phrases, symbols and other elements. Other preprocessing may be performed such as removing certain words or characters. The strings and/or tokens may be compared to other strings and/or tokens in other content stored in the hyperledger 118. In some embodiments, the strings and/or tokens may be compared to other content that pertains to the same content type or has a similar title, for example.

When the content 2700 includes a video, the audio of the video may be translated into text and the text may be processed as described above. Object character recognition may be used to identify objects in the video such that the objects may be compared with other videos stored in the hyperledger 118 to determine if there are matching objects between the videos. When the content 2700 includes images, the images may be compared with other images stored in content in the hyperledger 118 to determine if the images match.

In the content 2700 does not include at least a portion of new content relative to other content in the hyperledger 118, the rules 2306 may prevent the content 2700 from being stored on the hyperledger 118. If at least a portion of the content 2700 is new, then this particular rule may be satisfied. Using the particular rule may ensure that the knowledge and/or content that is added to the hyperledger 118 is not duplicated and may provide medical personnel entities the ability to own unique content in the hyperledger 118 to which they may control access.

In some embodiments, prior to allowing the content to be stored in the hyperledger 118, the one or more rules may include validating authenticating credentials (e.g., username, password, unique identifier) that are provided to the computer device 2310 during registration. The authenticating credentials may be included in the transaction request to perform the operation.

Responsive to determining that the one or more rules 2306 of the hyperledger 118 are satisfied, the method 2800 may involve performing the operation on the hyperledger 118 to store the content 2700 in the hyperledger 118. Responsive to determining that the one or more rules 2306 are not satisfied, the method 2800 may include preventing the operation from being performed on the hyperledger.

In some embodiments, the method 2800 may include receiving, from the computing device 2310, a second transaction request to perform a second operation on the hyperledger 118, where the second transaction request includes search criteria, and the second operation includes providing, based on the search criteria, a treatment plan pertaining to healthcare that is stored in the hyperledger 118. The method 2800 may also include executing the one or more rules of the hyperledger 118 to determine whether to allow the operation to be performed, where at least a second rule of the one or more rules includes determining whether the medical personnel entity has a right to access the content. Responsive to determining that the one or more rules 2306 of the hyperledger 118 are satisfied, the method 2800 may also include performing the second operation on the hyperledger to provide, based on the search criteria, the treatment plan pertaining to healthcare to the computing device 2310.

In some embodiments, the method 2800 may include receiving, from the computing device 2310, a second transaction request to perform a second operation on the hyperledger 118, where the second operation includes storing updated content pertaining to healthcare in the hyperledger 118, and the updated document adds additional content to original content included in the content 2700 stored in the hyperledger 118. The method 2800 may also include executing the one or more rules 2306 of the hyperledger 118 to determine whether to allow the operation to be performed, where at least a second rule of the one or more rules 2306 includes determining whether the additional content in the updated content is new relative to other content pertaining to healthcare stored in the hyperledger 118. Responsive to determining that the one or more rules 2306 of the hyperledger 118 are satisfied, the method 2800 may also include performing the second operation on the hyperledger 118 to store, in the hyperledger 118, the updated content including the additional content and the original content.

In some embodiments, a timestamp may be stored for the content 2700 in the hyperledger 118. Any time the content 2700 is updated and/or verified, a new timestamp may be stored with the content 2700, along with identifying information of the medical personnel entity that updated and/or verified the content 2700. This may enable a time series to be maintained for the content 2700 to allow a user to view the history of updates and/or verifications since the content 2700 was stored on the hyperledger 118.

In some embodiments, the method 2800 may include receiving, from a computing device associated with a second medical personnel entity, a second transaction request to perform a second operation on the hyperledger 118, where the second operation includes verifying the content 2700 in the hyperledger 118. The method 2800 may include determining whether the second medical personnel entity is associated with a valid authorizing credential (e.g., medical degree, medical license number, etc.) that is provided in the second transaction request or that is stored in the hyperledger 118. Responsive to determining that the second medical personnel entity is associated with the valid authorizing credential, the method 2800 may include performing the second operation on the hyperledger 118 by allowing the second medical personnel entity to verify the content 2700. A timestamp may be stored with the content 2700 when the content 2700 is verified.

In some embodiments, the method 2800 may include receiving, from a second computing device associated with a user, a transaction request to perform a second operation on the hyperledger 118, where the second operation includes determining whether the content 2700 is trustworthy. The method 2800 may include determining, using the hyperledger 118, a source of the content by identifying the medical personnel entity that authored or created the content 2700. The method 2800 may also include determining that the source is associated with one or more valid authorizing credentials (e.g., medical degrees, medical license numbers, etc.). The method 2800 may also include determining whether the content 2700 has been verified within a certain time period by a medical personnel entity with a valid authorizing credential. The method 2800 may also include providing a notification to the second computing device that the content is trustworthy based on the source of the content being associated with the authorizing credential and the content 2700 being verified within the certain time period.

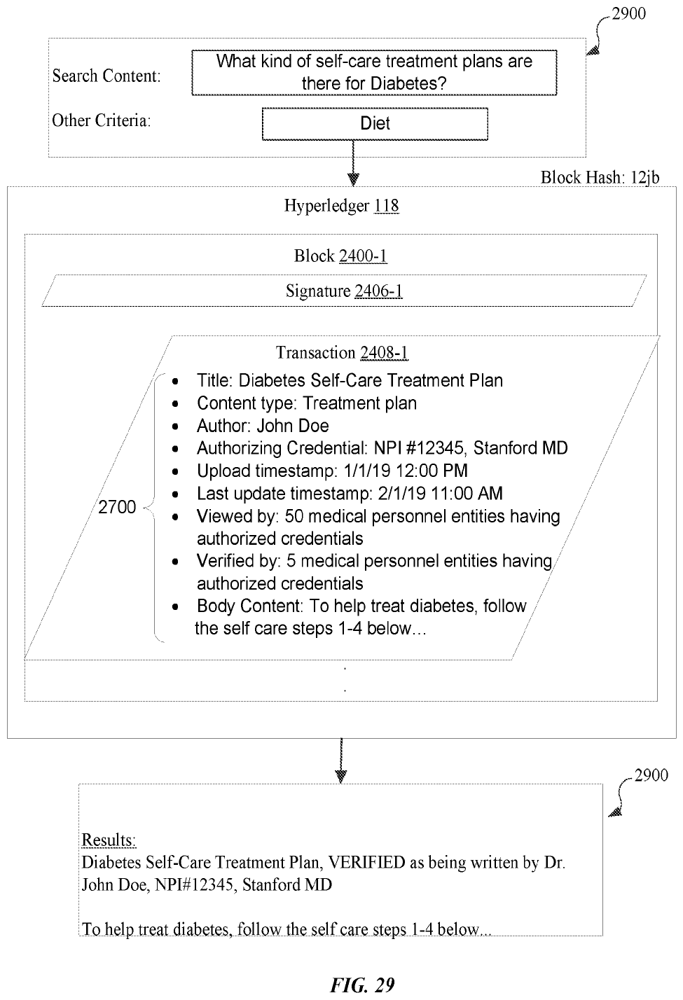

FIG. 29 shows an example search for content 2700, in accordance with various embodiments. As depicted, the content 2700 is stored in the transaction 2408-1 in the block 2400-1, which includes the signature 2406-1 of the medical personnel entity (e.g., John Doe) that authored and requested the content 2700 be stored in the hyperledger 118. A medical personnel entity may use a computing device to execute the software application that is logged into the cognitive intelligence platform 102 using the authenticating credentials. The software application may present a search user interface 2900 that includes various input elements, such as text bars that enable natural language text to be entered (e.g., questions, titles, authors, etc.) and/or other search criteria. The other search criteria may enable the user to select a particular diet, exercise, and/or medication that the medical personnel entity may choose for a particular patient having a particular medical condition. The search user interface 2900 may enable a user to search for content of interest that is stored on the hyperledger 118, to verify the source of searched for content, to verify that the source is associated with a valid authorizing credential, and/or to verify the content has been verified within a certain time period by a medical personnel entity having a valid authorizing credential, among other things.

In the depicted example, the user entered the question “What kind of self-care treatment plans are there for Diabetes?” and entered “Diet” as another search criteria. The computing device of the user may transmit a transaction request to the hyperledger 118 via the cognitive agent 110. The critical thinking engine 108, the natural language database 122, and/or the knowledge cloud 106 may be used to identify and understand the question asked in natural language. The hyperledger 118 may be searched, based on the question, to identify the content 2700 stored in transaction 2408-1. One or more rules 2306 may be executed to determine whether the requesting user has an access right to the content 2700. If the requesting user has the access right, then the content 2700 may be provided to the computing device of the requesting user and displayed on the search user interface 2900. If the requesting user does not have the access right, then a notification may be transmitted to the computing device of the requesting user prompting the user to purchase the access right.

When the content 2700 is transmitted to the computing device of the requesting user, the search user interface 2900 may present the content in a results section. For example, the results section may present the title “Diabetes Self-Care Treatment Plan”, as well as an indication that the content 2700 is verified as being written by Dr. John Doe, NPI #12345, Stanford MID. The results section may also present the body content “To help treat diabetes, follow the self care steps 1-4 below . . . ”

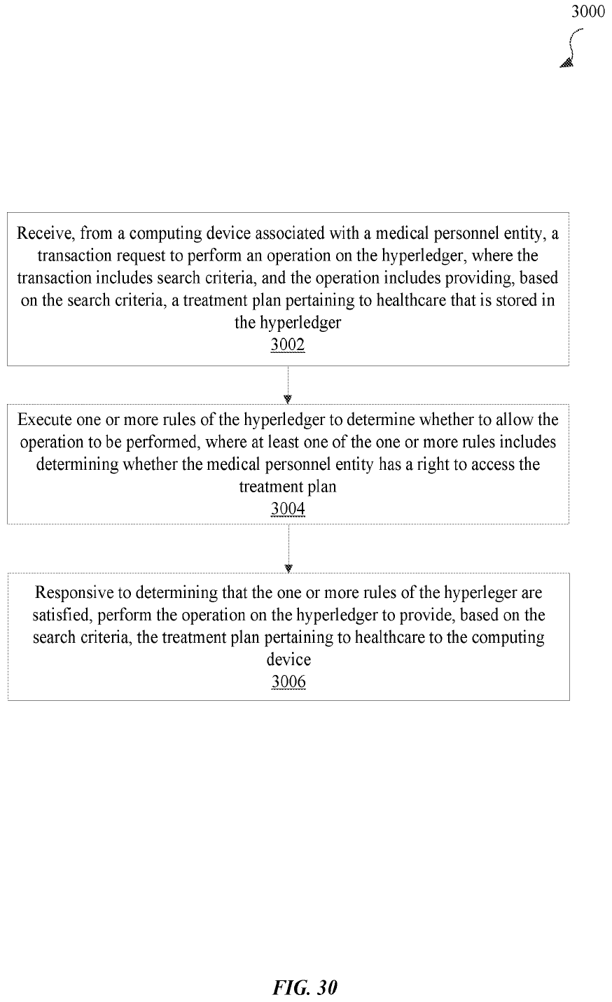

FIG. 30 shows a method 3000 for maintaining content pertaining to healthcare in a hyperledger 118, in accordance with various embodiments. In some embodiments, the method 3000 is implemented as a computer program product in a non-transitory computer-readable medium and executable by one or more processors of one or more computing devices described in the cognitive intelligence platform 102 of FIG. 1. In some embodiments, the method 3000 of FIG. 30 is implemented as a system for maintaining a hyperledger 118 storing content pertaining to healthcare at one or more nodes 116. The system can include components described in the cognitive intelligence platform 102.

In some embodiments, the method 3000 may involve receiving (3002), from a computing device 2310 associated with a medical personnel entity, a transaction request to perform an operation on the hyperledger 118, where the transaction includes search criteria, and the operation includes providing, based on the search criteria, content pertaining to healthcare that is stored in the hyperledger 118. In other embodiments, the search request may be for any suitable content (e.g., evidence-based guidelines, clinical studies, clinical trials, clinical techniques, knowledge representations (graphs), treatment plans, etc.) pertaining to healthcare.

The method 3000 may involve executing (3004) one or more rules of the hyperledger 118 to determine whether to allow the operation to be performed, where at least one of the one or more rules includes determining whether the medical personnel entity has a right to access the content. The requesting medical personnel entity may have a right to access the content 2700 when they purchase the right, such that the creator of the unique knowledge in the content 2700 may be rewarded for sharing the knowledge. In other embodiments, the requesting medical personnel entity may have a right to access the content 2700 when the requesting medical personnel entity is associated with an organization (e.g., a hospital) that has viewing privileges of the content 2700, the requesting medical personnel entity is associated with an authorizing credential pertaining to healthcare, the requesting medical personnel entity is associated with a same organization (e.g., hospital) as the creator of the content 2700, or some combination thereof.

Responsive to determining that the one or more rules of the hyperledger 118 are satisfied, the method 3000 may involve performing the operation on the hyperledger 118 to provide, based on the search criteria, the content 2700 pertaining to healthcare to the computing device 2310. Responsive to determining that the one or more rules of the hyperledger 118 are not satisfied, the method 3000 may not perform the operation.

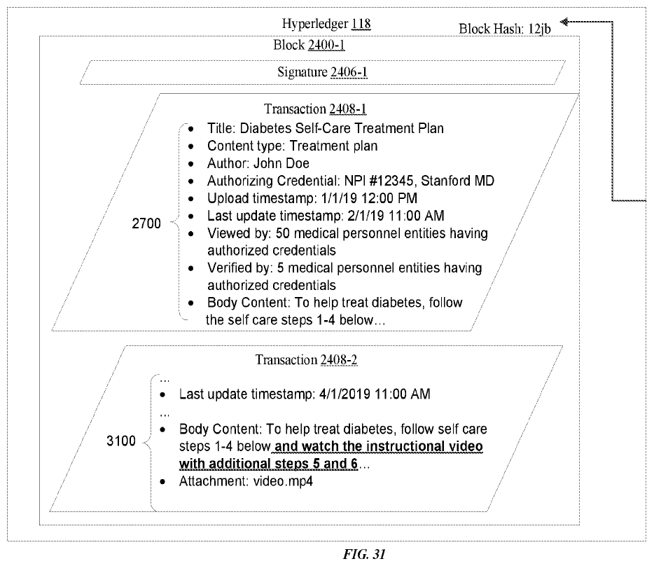

FIG. 31 shows an example updated content 3100 stored in the hyperledger 118, in accordance with various embodiments. A medical personnel entity may have used a computing device 2310 to submit a transaction request to perform an operation on the hyperledger 118, where the operation included storing the content 2700 on the hyperledger 118. One or more rules 2306 may have determined that the requesting medical personnel entity has (i) valid authenticating credentials and/or (ii) valid authorizing credentials, and/or that the content 2700 includes at least a portion of Body Content that is new relative to other content stored in the hyperledger 118. In the depicted example, the one or more rules 2306 are satisfied, and the content 2700 is stored in the transaction 2408-1 in the block 2406-1 in the hyperledger 118. Further, the block 2400-1 includes a signature 2406-1 of the medical personnel entity that provided the content 2700. The signature 2406-1 may be a digital signature that uniquely identifies the medical personnel entity as the source of the content 2700.

In the depicted example, the medical personnel entity may have used the computing device 2310 to submit a second transaction request to perform a second operation on the hyperledger 118, where the second operation included storing updated content 3100 on the hyperledger 118. Updated content 3100 may include original content (“To help treat diabetes, follow self care steps 1-4 below”), as well as additional content description and video. The additional description says “and watch the instructional video with additional steps 5 and 6” (which has been bolded and underlined).

One or more rules 2306 may have determined that the requesting medical personnel entity has (i) valid authenticating credentials and/or (ii) valid authorizing credentials, and/or that the content 3100 includes at least a portion of Body Content that is new relative to other content stored in the hyperledger 118. The additional description and video may be new relative to other content in the hyperledger 118. In the depicted example, the one or more rules 2306 are satisfied, and the content 3100 is stored in the transaction 2408-2 in the block 2406-1 in the hyperledger 118. Further, the block 2400-1 includes the signature 2406-1 of the medical personnel entity that provided the content 2700 and 3100. The signature 2406-1 may be a digital signature that uniquely identifies the medical personnel entity as the source of the content 2700 and 3100. In some embodiments, other medical personnel entities may provide the other content, which may be added to the block 2400-1 or other blocks 2400 if the one or more rules 2306 are satisfied, and a signature 2406 for those other medical personnel entities may be stored with their respective content.

In the depicted example, the updated content 3100 may be related to the additional content (represented by dotted line 2552) that included nodes representing additional content related to Type 2 Diabetes in the knowledge graph 2550 of FIG. 25B. Further, content 3100 may include a video 2612 that presents instructions for the new self care steps. The content 3100 may include various information, some of which is the same as the content 2700. Since the content 3100 is provided from the same medical personnel entity, John Doe, the author and authorizing credentials may be the same. The title and content type may remain the same, since the additional content adds on to the original content for the new self care treatment of Type 2 Diabetes. As depicted, the last update timestamp may be updated to a current timestamp for the update (“Apr. 1, 2019 11:00 AM”). Further, the Body Content section includes updated text, as noted above, and an Attachment includes the video file (“video.mp4”) that may be embedded in a document including the description of the treatment plan in the updated content 3100.

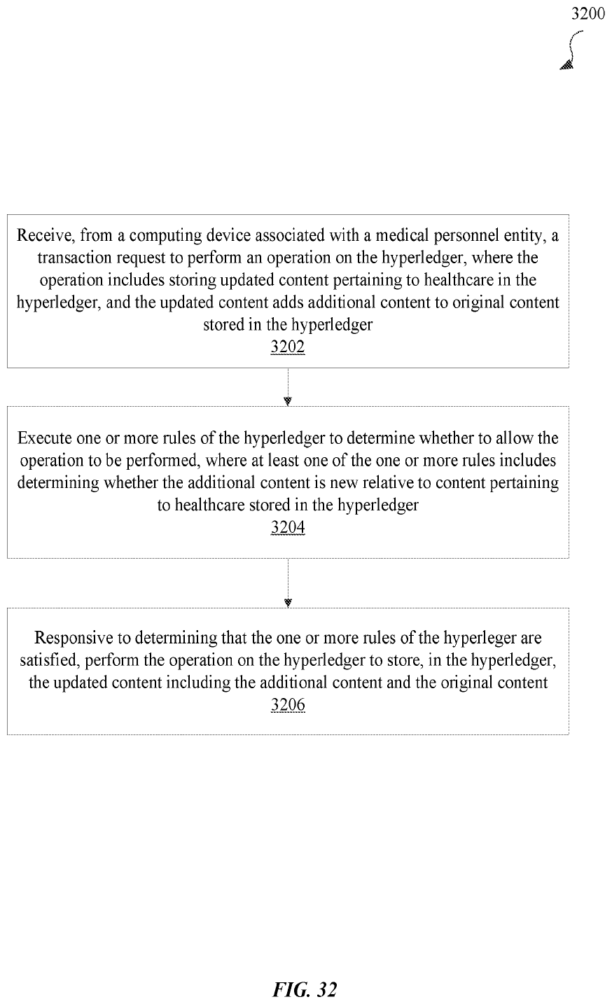

FIG. 32 shows a method 3200 for maintaining content pertaining to healthcare in a hyperledger 118, in accordance with various embodiments. In some embodiments, the method 3200 is implemented as a computer program product in a non-transitory computer-readable medium and executable by one or more processors of one or more computing devices described in the cognitive intelligence platform 102 of FIG. 1. In some embodiments, the method 3200 of FIG. 32 is implemented as a system for maintaining a hyperledger 118 storing content pertaining to healthcare at one or more nodes 116. The system can include components described in the cognitive intelligence platform 102.

In some embodiments, the method 3200 may involve receiving (3202), from a computing device 2310 associated with a medical personnel entity, a transaction request to perform an operation on the hyperledger 118, where the operation includes storing updated content 3100 pertaining to healthcare in the hyperledger 118, and the updated content 3100 adds additional content to original content stored in the hyperledger 118. The additional content may be any suitable text, video, or images describing or illustrating knowledge pertaining to healthcare (e.g., evidence-based guidelines, clinical processes, clinical trials, knowledge representations, etc.).

The method 3200 may involve executing (3204) one or more rules 2306 of the hyperledger 118 to determine whether to allow the operation to be performed, where at least one of the one or more rules includes determining whether the additional content is new relative to content pertaining to healthcare stored in the hyperledger 118. The one or more rules may identify the additional content in the updated content 3100 and preprocess the additional content as described above for videos, text strings, and/or images to determine whether the additional content is new relative to the other content in the hyperledger 118.

Responsive to determining that the one or more rules 2306 of the hyperledger 118 are satisfied, the method 3200 may involve performing the operation on the hyperledger 118 to store, in the hyperledger 118, the updated content 3100 including the additional content and the original content. A timestamp of when the updated content 3100 is stored in the hyperledger 118 may be stored with the updated content 3100, such that a time series of the evolution of the content may be maintained. The identity and the authorizing credentials of the medical personnel entity that provided the updated content 3100 may also be stored with the content 3100. Responsive to determining that the one or more rules 2306 of the hyperledger 118 are not satisfied, the operation to store the updated content 3100 may not be performed.

The various aspects, embodiments, implementations or features of the described embodiments can be used separately or in any combination. Various aspects of the described embodiments can be implemented by software, hardware or a combination of hardware and software. The described embodiments can also be embodied as computer readable code on a computer readable medium. The computer readable medium is any data storage device that can store data which can thereafter be read by a computer system. Examples of the computer readable medium include read-only memory, random-access memory, CD-ROMs, DVDs, magnetic tape, hard disk drives, solid-state drives, and optical data storage devices. The computer readable medium can also be distributed over network-coupled computer systems so that the computer readable code is stored and executed in a distributed fashion.

Consistent with the above disclosure, the examples of systems and method enumerated in the following clauses are specifically contemplated and are intended as a non-limiting set of examples.

Clause 1. A cognitive intelligence platform, comprising:
a first system configured to execute a knowledge cloud, the first system comprising:
   a first processor; and
   a first memory coupled to the first processor, the first memory storing instructions that cause the knowledge cloud to:
      receive inputs from medical facilities; and
      receive inputs from service providers;
a second system configured to implement a critical thinking engine, the critical thinking engine communicably coupled to the knowledge cloud, the second system comprising:
   a second processor; and
   a second memory coupled to the second processor, the second memory storing instructions that cause the critical thinking engine to receive inputs from the knowledge cloud; and
a third system configured to implement a cognitive agent, the cognitive agent communicably coupled to the critical thinking engine and the knowledge cloud, the third system comprising:
   a third processor; and
   a third memory coupled to the third processor, the third memory storing instructions that cause the cognitive agent to:
      receive an originating question from a user related to a subject matter;
      execute, using the critical thinking engine, a first round of analysis to generate an answer; and
      provide the answer to the user including a recommendation associated with the subject matter.

Clause 2. The cognitive intelligence platform of any preceding clause, wherein the second memory stores instructions that further cause the critical thinking engine to:
receive a first information;
receive a second information that contradicts the first information; and
process the first information and second information.

Clause 3. The cognitive intelligence platform of any preceding clause, wherein the second memory stores instructions that further cause the critical thinking engine to:
parse the originating question;
retrieve data from the knowledge cloud; and
perform a causal analysis of the data in view of the originating question, wherein the causal analysis, in part, informs the answer.

Clause 4. The cognitive intelligence platform of any preceding clause, wherein the second memory stores instructions that further cause the critical thinking engine to:
receive the originating question from the cognitive agent;
assess a first chain of logic associated with the originating question;
assess a second chain of logic associated with the originating question; and
provide the answer to the cognitive agent, wherein the answer is associated with the first chain of logic.

Clause 5. The cognitive intelligence platform of any preceding clause, wherein the third memory stores instructions that further cause the cognitive agent to communicate a logical argument that leads to a conclusion, wherein the conclusion, in part, informs the recommendation associated with the subject matter.

Clause 6. The cognitive intelligence platform of any preceding clause, wherein the third memory stores instructions that further cause the cognitive agent to:
render for display, to the user, a chain of logic that leads to the conclusion;
receive, from the user, an adjustment to the chain of logic; and
affect change in the critical thinking engine.

Clause 7. The cognitive intelligence platform of any preceding clause, wherein the third memory stores instructions that further cause the cognitive agent to:
render for display a micro survey;
receive data associated with the micro survey, wherein the data, in part, informs the recommendation associated with the subject matter.

Clause 8. The cognitive intelligence platform of any preceding clause, wherein when the cognitive agent provides the answer to the user, the third memory causes the cognitive agent to integrate data from at least three selected from the group consisting of: a micro survey, a physician's office, common sense knowledge, domain knowledge, an evidence-based medicine guideline, a clinical ontology, and curated medical advice.

Clause 9. A system comprising:
a knowledge cloud;
a critical thinking engine, the critical thinking engine communicably coupled to the knowledge cloud; and
a cognitive agent, the cognitive agent communicably coupled to the critical thinking engine and the knowledge cloud, wherein the cognitive agent is configured to interact with a user using natural language.

Clause 10. The system of any preceding clause, wherein the cognitive agent interacts with the user using at least one selected from the group consisting of: touch-based input, audio input, and typed input.

Clause 11. The system of claim any preceding clause, wherein the critical thinking engine is configured to:
receive a first information;
receive a second information that contradicts the first information; and
process the first information and the second information.

Clause 12. The system of any preceding clause, wherein the cognitive agent is configured to:
receive an originating question from the user related to a subject matter;
execute, using the critical thinking engine, a logical reasoning to generate an answer; and
provide the answer to the user including a recommendation associated with the subject matter.

Clause 13. The system of any preceding clause, wherein the critical thinking engine is configured to:
parse the originating question;
retrieve data from the knowledge cloud; and
perform a causal analysis of the data in view of the originating question, wherein the causal analysis, in part informs the answer.

Clause 14. The system of any preceding clause, wherein the critical thinking engine is configured to:
receive the originating question from the cognitive agent;
assess a first chain of logic associated with the originating question;
assess a second chain of logic associated with the originating question; and
provide the answer to the cognitive agent, wherein the answer is associated with the first chain of logic.

Clause 15. The system of any preceding clause, wherein the cognitive agent is further configured to render for display a chain of logic that leads to a conclusion, wherein the conclusion, in part, informs the answer.

Clause 16. A computer readable media storing instructions that are executable by a processor to cause a computer to execute operations comprising:
executing a cognitive intelligence platform that further comprises:
a knowledge cloud;
a critical thinking engine communicably coupled to the knowledge cloud; and
a cognitive agent communicably coupled to the critical thinking engine and the knowledge cloud, wherein the cognitive agent is configured to:
   receive an originating question from a user related to a subject matter;
   execute, using the critical thinking engine, a logical reasoning to generate an answer; and
   provide the answer to the user including a recommendation associated with the subject matter.

Clause 17. The computer-readable media of any preceding clause, wherein the cognitive agent executing within the cognitive intelligence platform is further configured to:
render for display a micro survey;
receive data associated with the micro survey, wherein the data, in part, informs the recommendation associated with the subject matter.

Clause 18. The computer-readable media of any preceding clause, wherein the critical thinking engine executing within the cognitive intelligence platform is further configured to:
receive the originating question from the cognitive agent;
assess a first chain of logic associated with the originating question to create a first answer;
assess a second chain of logic associated with the originating question to create a second answer, wherein the first answer contradicts the second answer; and
provide the first answer to the cognitive agent, wherein the first answer is the answer provided to the user.

Clause 19. The computer-readable media of any preceding clause, wherein the cognitive agent executing within the cognitive intelligence platform is further configured to render for display the first chain of logic to the user.

Clause 20. The computer-readable media of any preceding clause, wherein the cognitive agent executing within the cognitive intelligence platform is further configured to integrate data from at least three selected from the group consisting of: a micro survey, a physician's office, common sense knowledge, domain knowledge, an evidence-based medicine guideline, a clinical ontology, and curated medical advice.

Clause 21. A computer-implemented method for answering a user-generated natural language medical information query based on a diagnostic conversational template, the method comprising:
receiving a user-generated natural language medical information query at an artificial intelligence-based diagnostic conversation agent from a user interface on a mobile device;
responsive to content of the user-generated natural language medical information query, selecting a diagnostic fact variable set relevant to generating a medical advice query answer for the user-generated natural language medical information query by classifying the user-generated natural language medical information query into one of a set of domain-directed medical query classifications associated with respective diagnostic fact variable sets;
compiling user-specific medical fact variable values for one or more respective medical fact variables of the diagnostic fact variable set, wherein the compiling user-specific medical fact variable values for one or more respective medical fact variables of the diagnostic fact variable set further comprises:
   extracting a first set of user-specific medical fact variable values from a local user medical information profile associated with the user-generated natural language medical information query, and
   requesting a second set of user-specific medical fact variable values through natural-language questions sent to the user interface on the mobile device; and
responsive to the user-specific medical fact variable values, generating a medical advice query answer in response to the user-generated natural language medical information query.

Clause 22. The computer-implemented method for answering a user-generated natural language medical information query based on a diagnostic conversational template of any preceding clause, wherein the compiling user-specific medical fact variable values for one or more respective medical fact variables of the diagnostic fact variable set further comprises:
extracting a third set of user-specific medical fact variable values comprising lab result values from the local user medical information profile associated with the user-generated natural language medical information query.

Clause 23. The computer-implemented method for answering a user-generated natural language medical information query based on a diagnostic conversational template of any preceding clause, wherein the compiling user-specific medical fact variable values for one or more respective medical fact variables of the diagnostic fact variable set further comprises:
extracting a fourth set of user-specific medical fact variable values from a remote medical data service profile associated with the local user medical information profile.

Clause 24. The computer-implemented method for answering a user-generated natural language medical information query based on a diagnostic conversational template of any preceding clause, wherein the compiling user-specific medical fact variable values for one or more respective medical fact variables of the diagnostic fact variable set further comprises:
extracting a fifth set of user-specific medical fact variable values derived from demographic characterizations provided by a remote data service analysis of the local user medical information profile.

Clause 25. The computer-implemented method for answering a user-generated natural language medical information query based on a diagnostic conversational template of any preceding clause, wherein the generating the medical advice query answer in response to the user-generated natural language medical information query further comprises providing, in addition to text responsive to a medical question presented in the user-generated natural language medical information query, a treatment action-item recommendation responsive to user-specific medical fact variable values and non-responsive to the medical question presented in the user-generated natural language medical information query.

Clause 26. The computer-implemented method for answering a user-generated natural language medical information query based on a diagnostic conversational template of any preceding clause, wherein the generating the medical advice query answer in response to the user-generated natural language medical information query further comprises providing, in addition to text responsive to a medical question presented in the user-generated natural language medical information query, a medical education media resource responsive to the user-specific medical fact variable values and non-responsive to the medical question presented in the user-generated natural language medical information query.

Clause 27. The computer-implemented method for answering a user-generated natural language medical information query based on a diagnostic conversational template of any preceding clause, wherein selecting a diagnostic fact variable set relevant to generating a medical advice query answer for the user-generated natural language medical information query by classifying the user-generated natural language medical information query into one of a set of domain-directed medical query classifications associated with respective diagnostic fact variable set further comprises classifying the user-generated natural language medical information query into one of a set of domain-directed medical query classifications based on relevance to the local user medical information profile associated with the user-generated natural language medical information query.

Clause 28. A computer program product in a computer-readable medium for answering a user-generated natural language query, the computer program product in a computer-readable medium comprising program instructions which, when executed, cause a processor of a computer to perform:
receiving a user-generated natural language query at an artificial intelligence-based conversation agent from a user interface;
responsive to content of the user-generated natural language query, selecting a fact variable set relevant to generating a query answer for the user-generated natural language query by classifying the user-generated natural language query into one of a set of domain-directed query classifications associated with respective fact variable sets;
compiling user-specific fact variable values for one or more respective fact variables of the fact variable set; and
responsive to the fact variable values, generating the query answer in response to the user-generated natural language query.

Clause 29. The computer program product in a computer-readable medium for answering a user-generated natural language query of any preceding clause, wherein the program instructions which, when executed, cause the processor of the computer to perform compiling user-specific fact variable values for one or more respective fact variables of the fact variable set further comprise program instructions which, when executed, cause the computer program product to perform:
extracting a first set of user-specific fact variable values from a local user profile associated with the user-generated natural language query; and
requesting a second set of user-specific fact variable values through a conversational template comprising natural-language questions sent to the user interface on a mobile device.

Clause 30. The computer program product in a computer-readable medium for answering a user-generated natural language query of any preceding clause, wherein the program instructions which, when executed, cause the processor of the computer to perform compiling user-specific fact variable values for one or more respective fact variables of the fact variable set further comprise program instructions which, when executed, cause the computer program product to perform:
extracting a third set of user-specific fact variable values from a remote data service profile associated with the local user profile.

Clause 31. The computer program product in a computer-readable medium for answering a user-generated natural language query of any preceding clause, wherein the program instructions which, when executed, cause the processor of the computer to perform compiling user-specific fact variable values for one or more respective fact variables of the fact variable set further comprise program instructions which, when executed, cause the computer program product to perform:
extracting a fourth set of user-specific fact variable values derived from demographic characterizations provided by a remote data service analysis of the local user profile.

Clause 32. The computer program product in a computer-readable medium for answering a user-generated natural language query of any preceding clause, wherein program instructions which, when executed, cause the processor of the computer to perform the generating the query answer in response to the user-generated natural language query further comprise program instructions which, when executed, cause the processor of the computer to perform providing, in addition to text responsive to a question presented in the user-generated natural language query, an action-item recommendation responsive to the fact variable values and non-responsive to the question presented in the user-generated natural language query.

Clause 33. The computer program product in a computer-readable medium for answering a user-generated natural language query of any preceding clause, wherein the program instructions which, when executed, cause the processor of the computer to perform generating the query answer in response to the user-generated natural language query further comprise program instructions which, when executed, cause the processor of the computer to perform providing, in addition to text responsive to a question presented in the user-generated natural language query, an education media resource responsive to the fact variable values and non-responsive to the question presented in the user-generated natural language query.

Clause 34. The computer program product in a computer-readable medium for answering a user-generated natural language query of any preceding clause, wherein the program instructions which, when executed, cause the processor of the computer to perform selecting a fact variable set relevant to generating a query answer for the user-generated natural language query by classifying the user-generated natural language query into one of a set of domain-directed query classifications associated with respective fact variable sets further comprise program instructions which, when executed, cause the processor of the computer to perform classifying the user-generated natural language query into one of a set of domain-directed query classifications based on relevance to a local user profile associated with the user-generated natural language query.

Clause 35. A cognitive intelligence platform for answering a user-generated natural language query, the cognitive intelligence platform comprising:
a cognitive agent configured for receiving a user-generated natural language query at an artificial intelligence-based conversation agent from a user interface;
a critical thinking engine configured for, responsive to content of the user-generated natural language query, selecting a fact variable set relevant to generating a query answer for the user-generated natural language query by classifying the user-generated natural language query into one of a set of domain-directed query classifications associated with respective fact variable sets; and
a knowledge cloud compiling user-specific fact variable values for one or more respective fact variables of the fact variable set; and
wherein, responsive to the fact variable values, the cognitive agent is further configured for generating the query answer in response to the user-generated natural language query.

Clause 36. The cognitive intelligence platform of any preceding clause, wherein the knowledge cloud is further configured for:
extracting a first set of user-specific fact variable values from a local user profile associated with the user-generated natural language query; and
requesting a second set of user-specific fact variable values through a conversational template comprising natural-language questions sent to the user interface on a mobile device.

Clause 37. The cognitive intelligence platform of any preceding clause, wherein the knowledge cloud is further configured for:
extracting a third set of user-specific fact variable values from a remote data service profile associated with the local user profile.

Clause 38. The cognitive intelligence platform of any preceding clause, wherein the knowledge cloud is further configured for:
extracting a fourth set of user-specific fact variable values derived from demographic characterizations provided by a remote data service analysis of the local user profile.

Clause 39. The cognitive intelligence platform of any preceding clause, wherein cognitive agent is further configured for providing, in addition to text responsive to a question presented in the user-generated natural language query, an action-item recommendation responsive to the fact variable values and non-responsive to the question presented in the user-generated natural language query.

Clause 40. The cognitive intelligence platform of any preceding clause, wherein the critical thinking engine is further configured for providing, in addition to text responsive to a question presented in the user-generated natural language query, an education media resource responsive to the fact variable values and non-responsive to the question presented in the user-generated natural language query.

Clause 41. A computer-implemented method for answering a user-generated natural language query, the method comprising:
receiving a user-generated natural language query at an artificial intelligence-based conversation agent from a user interface;
responsive to content of the user-generated natural language query, selecting a fact variable set relevant to generating a query answer for the user-generated natural language query by classifying the user-generated natural language query into one of a set of domain-directed query classifications associated with respective fact variable sets;
compiling user-specific fact variable values for one or more respective fact variables of the fact variable set; and
responsive to the fact variable values, generating the query answer in response to the user-generated natural language query.

Clause 42. The method of any preceding clause, wherein the compiling user-specific fact variable values for one or more respective fact variables of the fact variable set further comprises:
extracting a first set of user-specific fact variable values from a local user profile associated with the user-generated natural language query; and
requesting a second set of user-specific fact variable values through a conversational template comprising natural-language questions sent to the user interface on a mobile device.

Clause 43. The method of any preceding clause, wherein the compiling user-specific fact variable values for one or more respective fact variables of the fact variable set further comprises:
extracting a third set of user-specific fact variable values from a remote data service profile associated with the local user profile.

Clause 44. The method of any preceding clause, wherein the compiling user-specific fact variable values for one or more respective fact variables of the fact variable set further comprises:
extracting a fourth set of user-specific fact variable values derived from demographic characterizations provided by a remote data service analysis of the local user profile.

Clause 45. The method of any preceding clause, wherein the generating the query answer in response to the user-generated natural language query further comprises providing, in addition to text responsive to a question presented in the user-generated natural language query, an action-item recommendation responsive to the fact variable values and non-responsive to the question presented in the user-generated natural language query.

Clause 46. The method of any preceding clause, wherein the generating the query answer in response to the user-generated natural language query further comprises providing, in addition to text responsive to a question presented in the user-generated natural language query, an education media resource responsive to the fact variable values and non-responsive to the question presented in the user-generated natural language query.

Clause 47. The method of any preceding clause, wherein selecting a fact variable set relevant to generating a query answer for the user-generated natural language query by classifying the user-generated natural language query into one of a set of domain-directed query classifications associated with respective fact variable sets further comprises classifying the user-generated natural language query into one of a set of domain-directed query classifications based on relevance to a local user profile associated with the user-generated natural language query.

Clause 48. A computer-implemented method for answering natural language medical information questions posed by a user of a medical conversational interface of a cognitive artificial intelligence system, the method comprising:
receiving from a medical conversational user interface a user-generated natural language medical information query at an artificial intelligence-based medical conversation cognitive agent;
extracting from the user-generated natural language medical information query a medical question from a user of the medical conversational user interface;
compiling a medical conversation language sample, wherein the medical conversation language sample comprises items of health-information-related-text derived from a health-related conversation between the artificial intelligence-based medical conversation cognitive agent and the user;
extracting from the medical conversation language sample internal medical concepts and medical data entities present within the medical conversation language sample, wherein the internal medical concepts comprise descriptions of medical attributes of the medical data entities;
inferring a therapeutic intent of the user from the internal medical concepts and the medical data entities;
generating a therapeutic paradigm logical framework for interpreting of the medical question, wherein
   the therapeutic paradigm logical framework comprises a catalog of medical logical progression paths from the medical question to respective therapeutic answers,
   each of the medical logical progression paths comprises one or more medical logical linkages from the medical question to a therapeutic path-specific answer, and
   the medical logical linkages comprise the internal medical concepts and external therapeutic paradigm concepts derived from a store of medical subject matter ontology data;
selecting a likely medical information path from among the medical logical progression paths to a likely path-dependent medical information answer based upon the therapeutic intent of the user; and
answering the medical question by following the likely medical information path to the likely path-dependent medical information answer.

Clause 49. The computer-implemented method for answering natural language medical information questions posed by a user of a medical conversational interface of a cognitive artificial intelligence system of any of any of the preceding clauses, further comprising relating medical inference groups of the internal medical concepts.

Clause 50. The computer-implemented method for answering natural language medical information questions posed by a user of a medical conversational interface of a cognitive artificial intelligence system of any of any of the preceding clauses, wherein the relating medical inference groups of the internal medical concepts further comprises relating groups of the internal medical concepts based at least in part on shared medical data entities for which each internal medical concept of a medical inference group of internal medical concepts describes a respective medical data attribute.

Clause 51. The computer-implemented method for answering natural language medical information questions posed by a user of a medical conversational interface of a cognitive artificial intelligence system of any of the preceding clauses, wherein selecting a likely medical information path from among the medical logical progression paths to a likely path-dependent medical information answer based upon the intent further comprises selecting a likely medical information path from among the medical logical progression paths to a likely path-dependent medical information answer based in part upon the therapeutic intent of the user and in part upon sufficiency of medical diagnostic data to complete the medical logical linkages.

Clause 52. The computer-implemented method for answering natural language medical information questions posed by a user of a medical conversational interface of a cognitive artificial intelligence system of any of the preceding clauses, wherein selecting a likely medical information path from among the medical logical progression paths to a likely path-dependent medical information answer based upon the intent further comprises selecting a likely medical information path from among the medical logical progression paths to a likely path-dependent medical information answer after requesting additional medical diagnostic data from the user.

Clause 53. The computer-implemented method for answering natural language medical information questions posed by a user of a medical conversational interface of a cognitive artificial intelligence system of any of the preceding clauses, wherein selecting a likely medical information path from among the medical logical progression paths to a likely path-dependent medical information answer based upon the intent further comprises selecting a likely medical information path from among the medical logical progression paths to a likely path-dependent medical information answer based in part upon treatment sub-intents comprising tactical constituents related to the therapeutic intent of the user by the store of medical subject matter ontology data.

Clause 54. The computer-implemented method for answering natural language medical information questions posed by a user of a medical conversational interface of a cognitive artificial intelligence system of any of the preceding clauses, wherein selecting a likely medical information path from among the medical logical progression paths to a likely path-dependent medical information answer based upon the intent further comprises selecting a likely medical information path from among the medical logical progression paths to a likely path-dependent medical information answer based in part upon the therapeutic intent of the user and in part upon sufficiency of medical diagnostic data to complete the medical logical linkages, wherein the medical diagnostic data to complete the medical logical linkages includes user-specific medical diagnostic data.

Clause 55. A cognitive intelligence platform for answering natural language questions posed by a user of a conversational interface of an artificial intelligence system, the cognitive intelligence platform comprising:
a cognitive agent configured for receiving from a user interface a user-generated natural language query, wherein the cognitive agent is an artificial intelligence-based conversation agent;
a knowledge cloud containing a store of subject matter ontology data;
a critical thinking engine configured for:
   extracting from the user-generated natural language query a question from a user of the user interface,
   compiling a language sample, wherein the language sample comprises items of text derived from a conversation between the artificial intelligence-based conversation agent and the user,
   extracting from the language sample internal concepts and entities present within the language sample, wherein the internal concepts comprise descriptions of attributes of the entities,
   inferring an intent of the user from the internal concepts and the entities,
   generating a logical framework for interpreting of the question, wherein
      the logical framework comprises a catalog of paths from the question to respective answers,
      each of the paths comprises one or more linkages from the question to a path-specific answer, and
      the linkages comprise the internal concepts and external concepts derived from the store of subject matter ontology data,
   selecting a likely path from among the paths to a likely path-dependent answer based upon the intent, and
   answering the question by following the likely path to the likely path-dependent answer.

Clause 56. The cognitive intelligence platform for answering natural language questions posed by a user of a conversational interface of an artificial intelligence system of any of the preceding clauses, wherein the critical thinking engine is further configured for relating groups of the internal concepts.

Clause 57. The cognitive intelligence platform for answering natural language questions posed by a user of a conversational interface of an artificial intelligence system of any of the preceding clauses, wherein the critical thinking engine is further configured for relating groups of the internal concepts by relating groups of the internal concepts based at least in part on shared entities for which each internal concept of a group of internal concepts describes a respective attribute.

Clause 58. The cognitive intelligence platform for answering natural language questions posed by a user of a conversational interface of an artificial intelligence system of any of the preceding clauses, wherein the critical thinking engine is further configured for selecting a likely path from among the paths to a likely path-dependent answer based upon the intent further comprises selecting a likely path from among the paths to a likely path-dependent answer based in part upon the intent and in part upon sufficiency of data to complete the linkages.

Clause 59. The cognitive intelligence platform for answering natural language questions posed by a user of a conversational interface of an artificial intelligence system of any of the preceding clauses, wherein the critical thinking engine is further configured for selecting a likely path from among the paths to a likely path-dependent answer based upon the intent further comprises selecting a likely path from among the paths to a likely path-dependent answer after requesting additional data from the user.

Clause 60. The cognitive intelligence platform for answering natural language questions posed by a user of a conversational interface of an artificial intelligence system of 8, wherein the critical thinking engine is further configured for selecting a likely path from among the paths to a likely path-dependent answer based upon the intent further comprises selecting a likely path from among the paths to a likely path-dependent answer based in part upon sub-intents comprising tactical constituents related to the intent by the store of subject matter ontology data.

Clause 61. The cognitive intelligence platform for answering natural language questions posed by a user of a conversational interface of an artificial intelligence system of any of the preceding clauses, wherein the critical thinking engine is further configured for selecting a likely path from among the paths to a likely path-dependent answer based upon the intent further comprises selecting a likely path from among the paths to a likely path-dependent answer based in part upon the intent and in part upon sufficiency of data to complete the linkages, wherein the data to complete the linkages includes user-specific data.

Clause 62. A computer program product in a computer-readable medium for answering natural language questions posed by a user of a conversational interface of an artificial intelligence system, the computer program product in a computer-readable medium comprising instructions, which, when executed, cause a processor of a computer to perform:
receiving from a user interface a user-generated natural language query at an artificial intelligence-based conversation agent;
extracting from the user-generated natural language query a question from a user of the user interface;
compiling a language sample, wherein the language sample comprises items of text derived from a conversation between the artificial intelligence-based conversation agent and the user;
extracting from the language sample internal concepts and entities present within the language sample, wherein the internal concepts comprise descriptions of attributes of the entities;
inferring an intent of the user from the internal concepts and the entities;
generating a logical framework for interpreting of the question, wherein
   the logical framework comprises a catalog of paths from the question to respective answers,
   each of the paths comprises one or more linkages from the question to a path-specific answer, and
   the linkages comprise the internal concepts and external concepts derived from a store of subject matter ontology data;
selecting a likely path from among the paths to a likely path-dependent answer based upon the intent; and
answering the question by following the likely path to the likely path-dependent answer.

Clause 63. The computer program product in a computer-readable medium for answering natural language questions posed by a user of a conversational interface of an artificial intelligence system of any of the preceding clauses, further comprising instructions, which, when executed, cause the processor of the computer to perform relating groups of the internal concepts.

Clause 64. The computer program product in a computer-readable medium for answering natural language questions posed by a user of a conversational interface of an artificial intelligence system of any of the preceding clauses, wherein the instructions, which, when executed, cause the processor of the computer to perform relating groups of the internal concepts further comprise instructions, which, when executed, cause the processor of the computer to perform relating groups of the internal concepts based at least in part on shared entities for which each internal concept of a group of internal concepts describes a respective attribute.

Clause 65. The computer program product in a computer-readable medium for answering natural language questions posed by a user of a conversational interface of an artificial intelligence system of any of the preceding clauses, wherein the instructions, which, when executed, cause the processor of the computer to perform selecting a likely path from among the paths to a likely path-dependent answer based upon the intent further comprise instructions, which, when executed, cause the processor of the computer to perform selecting a likely path from among the paths to a likely path-dependent answer based in part upon the intent and in part upon sufficiency of data to complete the linkages.

Clause 66. The computer program product in a computer-readable medium for answering natural language questions posed by a user of a conversational interface of an artificial intelligence system of any of the preceding clauses, wherein instructions, which, when executed, cause the processor of the computer to perform selecting a likely path from among the paths to a likely path-dependent answer based upon the intent further comprise instructions, which, when executed, cause the processor of the computer to perform selecting a likely path from among the paths to a likely path-dependent answer after requesting additional data from the user.

Clause 67. The computer program product in a computer-readable medium for answering natural language questions posed by a user of a conversational interface of an artificial intelligence system of any of the preceding clauses, wherein the instructions, which, when executed, cause the processor of the computer to perform selecting a likely path from among the paths to a likely path-dependent answer based upon the intent further comprise instructions, which, when executed, cause the processor of the computer to perform selecting a likely path from among the paths to a likely path-dependent answer based in part upon sub-intents comprising tactical constituents related to the intent by the store of subject matter ontology data.

Clause 68. A method for answering natural language questions posed by a user of a conversational interface of an artificial intelligence system, the method comprising:
receiving from a user interface a user-generated natural language query at an artificial intelligence-based conversation agent;
extracting from the user-generated natural language query a question from a user of the user interface;
compiling a language sample, wherein the language sample comprises items of text derived from a conversation between the artificial intelligence-based conversation agent and the user;
extracting from the language sample internal concepts and entities present within the language sample, wherein the internal concepts comprise descriptions of attributes of the entities;
inferring an intent of the user from the internal concepts and the entities;
generating a logical framework for interpreting of the question, wherein
   the logical framework comprises a catalog of paths from the question to respective answers,
   each of the paths comprises one or more linkages from the question to a path-specific answer, and
   the linkages comprise the internal concepts and external concepts derived from a store of subject matter ontology data;
selecting a likely path from among the paths to a likely path-dependent answer based upon the intent; and
answering the question by following the likely path to the likely path-dependent answer.

Clause 69. The method for answering natural language questions posed by a user of a conversational interface of an artificial intelligence system of any of the preceding clauses, further comprising relating groups of the internal concepts.

Clause 70. The method for answering natural language questions posed by a user of a conversational interface of an artificial intelligence system of any of the preceding clauses, wherein the relating groups of the internal concepts further comprises relating groups of the internal concepts based at least in part on shared entities for which each internal concept of a group of internal concepts describes a respective attribute.

Clause 71. The method for answering natural language questions posed by a user of a conversational interface of an artificial intelligence system of any of the preceding clauses, wherein selecting a likely path from among the paths to a likely path-dependent answer based upon the intent further comprises selecting a likely path from among the paths to a likely path-dependent answer based in part upon the intent and in part upon sufficiency of data to complete the linkages.

Clause 72. The method for answering natural language questions posed by a user of a conversational interface of an artificial intelligence system of any of the preceding clauses, wherein selecting a likely path from among the paths to a likely path-dependent answer based upon the intent further comprises selecting a likely path from among the paths to a likely path-dependent answer after requesting additional data from the user.

Clause 73. The method for answering natural language questions posed by a user of a conversational interface of an artificial intelligence system of any of the preceding clauses, wherein selecting a likely path from among the paths to a likely path-dependent answer based upon the intent further comprises selecting a likely path from among the paths to a likely path-dependent answer based in part upon sub-intents comprising tactical constituents related to the intent by the store of subject matter ontology data.

Clause 74. The method for answering natural language questions posed by a user of a conversational interface of an artificial intelligence system of any of the preceding clauses, wherein selecting a likely path from among the paths to a likely path-dependent answer based upon the intent further comprises selecting a likely path from among the paths to a likely path-dependent answer based in part upon the intent and in part upon sufficiency of data to complete the linkages, wherein the data to complete the linkages includes user-specific data.

Clause 75. A computer-implemented method for providing therapeutic medical action recommendations in response to a medical information natural language conversation stream, the computer-implemented method comprising:
receiving segments of a medical information natural language conversation stream at an artificial intelligence-based health information conversation agent from a medical information conversation user interface;
responsive to medical information content of a user medical information profile associated with the medical information natural language conversation stream, defining a desired clinical management outcome objective relevant to health management criteria and related health management data attributes of the user medical information profile;
identifying a set of potential therapeutic interventions correlated to advancement of the clinical management outcome objective;
selecting from among the set of potential therapeutic interventions correlated to advancement of the clinical management outcome objective a medical intervention likely to advance the clinical management outcome objective;
presenting in the medical information natural language conversation stream a therapeutic advice conversation stream segment designed to stimulate execution of the medical intervention likely to advance the clinical management outcome objective; and presenting to the user in the medical information natural language conversation stream a therapeutic advice conversation stream segment explaining a correlation between the medical intervention likely to advance the clinical management outcome objective and achievement of the clinical management outcome objective.

Clause 76. The computer-implemented method for providing therapeutic medical action recommendations in response to a medical information natural language conversation stream of any preceding clause, wherein the selecting from among the set of potential therapeutic interventions correlated to advancement of the clinical management outcome objective a medical intervention likely to advance the clinical management outcome objective further comprises:
selecting from among the set of potential therapeutic interventions correlated to advancement of the clinical management outcome objective the medical intervention likely to advance the clinical management outcome objective based on a set of factors comprising likelihood of patient compliance with the a recommendation for the a medical intervention likely to advance the clinical management outcome objective and a statistical likelihood that the action will materially advance the clinical management outcome objective. Clause 77. The computer-implemented method for providing therapeutic medical action recommendations in response to a medical information natural language conversation stream any preceding clause, wherein the presenting to the user in the medical information natural language conversation stream a therapeutic advice conversation stream segment designed to stimulate execution of the action likely to advance the clinical management outcome objective further comprises presenting to the user in the medical information natural language conversation stream a therapeutic advice conversation stream segment explaining a cost-benefit analysis comparing likely results of performance of the action likely to advance the clinical management outcome objective and likely results of non-performance of the action likely to advance the clinical management outcome objective.

Clause 78. The computer-implemented method for providing therapeutic medical action recommendations in response to a medical information natural language conversation stream of any preceding clause, wherein the selecting from among the set of potential therapeutic interventions correlated to advancement of the clinical management outcome objective a medical intervention likely to advance the clinical management outcome objective further comprises:
selecting from among the set of potential therapeutic interventions correlated to advancement of the clinical management outcome objective the medical intervention likely to advance the clinical management outcome objective based on a set of factors comprising likelihood total expected cost expectation associated with the recommendation for the a medical intervention likely to advance the clinical management outcome objective.

Clause 79. The computer-implemented method for providing therapeutic medical action recommendations in response to a medical information natural language conversation stream of any preceding clause, wherein the presenting to the user in the medical information natural language conversation stream a therapeutic advice conversation stream segment designed to stimulate execution of the action likely to advance the clinical management outcome objective further comprises presenting to the user in the medical information natural language conversation stream a conversation stream reinforcing the recommendation after expiration of a delay period.

Clause 80. The computer-implemented method for providing therapeutic medical action recommendations in response to a medical information natural language conversation stream of any preceding clause, wherein the presenting to the user in the medical information natural language conversation stream a therapeutic advice conversation stream segment designed to stimulate execution of the action likely to advance the clinical management outcome objective further comprises presenting to the user in the medical information natural language conversation stream a therapeutic advice conversation stream segment explaining reasons for selection of the clinical management outcome objective.

Clause 81. The computer-implemented method for providing therapeutic medical action recommendations in response to a medical information natural language conversation stream of any preceding clause, wherein the presenting to the user in the medical information natural language conversation stream a therapeutic advice conversation stream segment designed to stimulate execution of the action likely to advance the clinical management outcome objective further comprises notifying third party service providers of the clinical management outcome objective and the recommendation.

Clause 82. A computer program product in a non-transitory computer-readable medium for providing therapeutic medical action recommendations in response to a medical information natural language conversation stream, the computer program product in a non-transitory computer-readable medium comprising instructions which, when executed cause a processor of a computer to perform:
receiving segments of a medical information natural language conversation stream at an artificial intelligence-based health information conversation agent from a medical information conversation user interface;
responsive to medical information content of a user medical information profile associated with the medical information natural language conversation stream, defining a clinical management outcome objective relevant to health management criteria and related health management data attributes of the profile;
selecting a medical intervention likely to advance the clinical management outcome objective; and
presenting to the user in the medical information natural language conversation stream a therapeutic advice conversation stream segment designed to stimulate execution of the action likely to advance the clinical management outcome objective.

83. The computer program product in a non-transitory computer-readable medium of any preceding clause, wherein the instructions which, when executed cause the processor of the computer to perform selecting a medical intervention likely to advance the clinical management outcome objective further comprise instructions which, when executed cause the processor of the computer to perform:
identifying a set of potential therapeutic interventions correlated to advancement of the clinical management outcome objective; and
selecting the action likely to advance the user outcome objective based on a set of factors comprising likelihood of performance of the action likely to advance the user outcome objective and likelihood that the action will materially advance the user outcome objective.

Clause 84. The computer program product in a non-transitory computer-readable medium of any preceding clause, wherein the instructions which, when executed cause the processor of the computer to perform presenting to the user in the medical information natural language conversation stream a therapeutic advice conversation stream segment designed to stimulate execution of the action likely to advance the clinical management outcome objective further comprise instructions which, when executed cause the processor of the computer to perform presenting to the user in the medical information natural language conversation stream a therapeutic advice conversation stream segment explaining a correlation between the action likely to advance the clinical management outcome objective and achievement of the clinical management outcome objective.

Clause 85. The computer program product in a non-transitory computer-readable medium of any preceding clause, wherein the instructions which, when executed cause the processor of the computer to perform presenting to the user in the medical information natural language conversation stream a therapeutic advice conversation stream segment designed to stimulate execution of the action likely to advance the clinical management outcome objective further comprise instructions which, when executed cause the processor of the computer to perform presenting to the user in the medical information natural language conversation stream a therapeutic advice conversation stream segment explaining a plan of subsequent actions likely to advance the clinical management outcome objective.

Clause 86. The computer program product in a non-transitory computer-readable medium of any preceding clause, wherein the instructions which, when executed cause the processor of the computer to perform presenting to the user in the medical information natural language conversation stream a therapeutic advice conversation stream segment designed to stimulate execution of the action likely to advance the clinical management outcome objective further comprise instructions which, when executed cause the processor of the computer to perform presenting to the user in the medical information natural language conversation stream a conversation stream reinforcing the recommendation after expiration of a delay period.

Clause 87. The computer program product in a non-transitory computer-readable medium of any preceding clause, wherein the instructions which, when executed cause the processor of the computer to perform presenting to the user in the medical information natural language conversation stream a therapeutic advice conversation stream segment designed to stimulate execution of the action likely to advance the clinical management outcome objective further comprise instructions which, when executed cause the processor of the computer to perform presenting to the user in the medical information natural language conversation stream a therapeutic advice conversation stream segment explaining reasons for selection of the clinical management outcome objective.

Clause 88. The computer program product in a non-transitory computer-readable medium of any preceding clause, wherein the instructions which, when executed cause the processor of the computer to perform presenting to the user in the medical information natural language conversation stream a therapeutic advice conversation stream segment designed to stimulate execution of the action likely to advance the clinical management outcome objective further comprise instructions which, when executed cause the processor of the computer to perform notifying third party service providers of the clinical management outcome objective and the recommendation.

Clause 89. A system for providing therapeutic medical action recommendations in response to a medical information natural language conversation stream, the system comprising:
a knowledge cloud configured for receiving segments of a medical information natural language conversation stream at an artificial intelligence-based health information from a medical information conversation user interface of a cognitive agent;
a critical thinking engine configured for:
   responsive to medical information content of a user medical information profile associated with the medical information natural language conversation stream in the knowledge cloud, defining a clinical management outcome objective relevant to health management criteria and related health management data attributes of the profile, and
   selecting a medical intervention likely to advance the clinical management outcome objective; and
the cognitive agent, wherein the cognitive agent is configure for presenting to the user in the medical information natural language conversation stream a therapeutic advice conversation stream segment designed to stimulate execution of the action likely to advance the clinical management outcome objective.

Clause 90. The system of any preceding clause, wherein the selecting a medical intervention likely to advance the clinical management outcome objective further comprises:
identifying a set of potential therapeutic interventions correlated to advancement of the clinical management outcome objective; and
selecting the action likely to advance the user outcome objective based on a set of factors comprising likelihood of performance of the action likely to advance the user outcome objective and likelihood that the action will materially advance the user outcome objective.

Clause 91. The system of claim any preceding clause, wherein the presenting to the user in the medical information natural language conversation stream a therapeutic advice conversation stream segment designed to stimulate execution of the action likely to advance the clinical management outcome objective further comprises presenting to the user in the medical information natural language conversation stream a therapeutic advice conversation stream segment explaining a correlation between the action likely to advance the clinical management outcome objective and achievement of the clinical management outcome objective.

Clause 92. The system of any preceding clause, wherein the presenting to the user in the medical information natural language conversation stream a therapeutic advice conversation stream segment designed to stimulate execution of the action likely to advance the clinical management outcome objective further comprises presenting to the user in the medical information natural language conversation stream a therapeutic advice conversation stream segment explaining a plan of subsequent actions likely to advance the clinical management outcome objective.

Clause 93. The system of any preceding clause, wherein the presenting to the user in the medical information natural language conversation stream a therapeutic advice conversation stream segment designed to stimulate execution of the action likely to advance the clinical management outcome objective further comprises presenting to the user in the medical information natural language conversation stream a conversation stream reinforcing the recommendation after expiration of a delay period.

Clause 94. The system of any preceding clause, wherein the presenting to the user in the medical information natural language conversation stream a conversation stream segment designed to stimulate execution of the action likely to advance the clinical management outcome objective further comprises presenting to the user in the medical information natural language conversation stream a conversation stream segment explaining reasons for selection of the clinical management outcome objective.

Clause 95. A computer-implemented method for providing action recommendations in response to a user-generated natural language conversation stream, the method comprising:
receiving segments of a user-generated natural language conversation stream at an artificial intelligence-based conversation agent from a user interface;
responsive to content of a user profile associated with the user-generated natural language conversation stream, defining a user action outcome objective relevant to attributes of the profile;
selecting an action likely to advance the user action outcome objective; and
presenting to the user in the user-generated natural language conversation stream a conversation stream segment designed to motivate performance of the action likely to advance the user action outcome objective.

Clause 96. The method of any preceding clause, wherein the selecting an action likely to advance the user action outcome objective further comprises:
identifying a set of actions correlated to advancement of the user action outcome objective; and
selecting the action likely to advance the user outcome objective based on a set of factors comprising likelihood of performance of the action likely to advance the user outcome objective and likelihood that the action will materially advance the user outcome objective.

Clause 97. The method of any preceding clause, wherein the presenting to the user in the user-generated natural language conversation stream a conversation stream segment designed to motivate performance of the action likely to advance the user action outcome objective further comprises presenting to the user in the user-generated natural language conversation stream a conversation stream segment explaining a correlation between the action likely to advance the user action outcome objective and achievement of the user action outcome objective.

Clause 98. The method of any preceding clause, wherein the presenting to the user in the user-generated natural language conversation stream a conversation stream segment designed to motivate performance of the action likely to advance the user action outcome objective further comprises presenting to the user in the user-generated natural language conversation stream a conversation stream segment explaining a plan of subsequent actions likely to advance the user action outcome objective.

Clause 99. The method of any preceding clause, wherein the presenting to the user in the user-generated natural language conversation stream a conversation stream segment designed to motivate performance of the action likely to advance the user action outcome objective further comprises presenting to the user in the user-generated natural language conversation stream a conversation stream reinforcing the recommendation after expiration of a delay period.

Clause 100. The method of any preceding clause, wherein the presenting to the user in the user-generated natural language conversation stream a conversation stream segment designed to motivate performance of the action likely to advance the user action outcome objective further comprises presenting to the user in the user-generated natural language conversation stream a conversation stream segment explaining reasons for selection of the user action outcome objective.

Clause 101. The method of any preceding clause, wherein the presenting to the user in the user-generated natural language conversation stream a conversation stream segment designed to motivate performance of the action likely to advance the user action outcome objective further comprises notifying third party service providers of the user action outcome objective and the recommendation.

Clause 102. A method for maintaining content pertaining to healthcare in a hyperledger, the method comprising:
receiving, from a computing device associated with a medical personnel entity, a transaction request to perform an operation on the hyperledger, wherein the operation comprises storing content pertaining to healthcare in the hyperledger;
executing one or more rules of the hyperledger to determine whether to allow the operation to be performed, wherein at least one of the one or more rules comprises determining whether the medical personnel entity is associated with an authorizing credential pertaining to healthcare; and
responsive to determining that the one or more rules of the hyperledger are satisfied, performing the operation on the hyperledger to store the content in the hyperledger.

Clause 103. The method of claim 1, wherein the authorizing credential is stored at a transaction in the hyperledger or the authorizing credential is included in the transaction request.

Clause 104. The method of claim 1, wherein the one or more rules comprise determining, using one or more transactions stored in the hyperledger, whether at least a portion of the content is new relative to other content in the hyperledger.

Clause 105. The method of claim 1, wherein the authorizing credential comprises a national provider identifier, and the one or more rules comprise determining, using one or more transactions stored in the hyperledger, whether the authorizing credential is valid.

Clause 106. The method of claim 1, further comprising validating authenticating credentials, authorizing credentials, or both prior to allowing the content to be stored in the hyperledger, wherein the authenticating credentials are provided to the computing device during registration and the authorizing credentials pertain to a medical license.

Clause 107. The method of claim 1, further comprising, responsive to determining that the one or more rules of the hyperledger are not satisfied, preventing the operation from being performed on the hyperledger.

Clause 108. The method of claim 1, wherein the content comprises a treatment plan, wherein at least a portion of the treatment plan is written by the medical personnel entity.

Clause 109. The method of claim 1, further comprising:
receiving, from the computing device, a second transaction request to perform a second operation on the hyperledger, wherein the second transaction request comprises search criteria, and the second operation comprises providing, based on the search criteria, a treatment plan pertaining to healthcare that is stored in the hyperledger;
executing the one or more rules of the hyperledger to determine whether to allow the operation to be performed, wherein at least a second rule of the one or more rules comprises determining whether the medical personnel entity has a right to access the content; and
responsive to determining that the one or more rules of the hyperledger are satisfied, performing the second operation on the hyperledger to provide, based on the search criteria, the treatment plan pertaining to healthcare to the computing device.

Clause 110. The method of claim 1, further comprising:
receiving, from the computing device associated, a second transaction request to perform a second operation on the hyperledger, wherein the second operation comprises storing updated content pertaining to healthcare in the hyperledger, and the updated document adds additional content to original content included in the content stored in the hyperledger;
executing the one or more rules of the hyperledger to determine whether to allow the operation to be performed, wherein at least a second rule of the one or more rules comprises determining whether the additional content in the updated content is new relative to other content pertaining to healthcare stored in the hyperledger; and
responsive to determining that the one or more rules of the hyperledger are satisfied, performing the second operation on the hyperledger to store, in the hyperledger, the updated content including the additional content and the original content.

Clause 111. The method of claim 1, further comprising maintaining a time series for the content that indicates when the content was stored, modified, validated, or some combination thereof.

Clause 112. The method of claim 1, further comprising:
receiving, from a computing device associated with a second medical personnel entity, a second transaction request to perform a second operation on the hyperledger, wherein the second operation comprises verifying the content in the hyperledger;
determining whether the second medical personnel entity is associated with a valid authorizing credential; and
responsive to determining that the second medical personnel entity is associated with the valid authoring credential, performing the second operation on the hyperledger by allowing the second medical personnel entity to verify the content.

Clause 113. The method of claim 1, further comprising:
receiving, from a second computing device, a transaction request to perform a second operation on the hyperledger, wherein the second operation comprises determining whether the content is trustworthy;
determining, using the hyperledger, a source of the content;
determining that the source is associated with the authorizing credential;
determining whether the content has been verified within a certain time period; and
providing a notification to the second computing device that the content is trustworthy based on the source of the content being associated with the authorizing credential and the content being verified within the certain time period.

Clause 114. The method of claim 1, wherein the content comprises a treatment plan for a medical issue, the medical personnel entity is a licensed medical doctor, and the hyperledger maintains different treatment plans for different licensed medical doctors that are validated as being provided by licensed medical doctors based on authorizing credentials that are stored in the hyperledger.

Clause 115. A method for maintaining content pertaining to healthcare in a hyperledger, the method comprising:
receiving, from a computing device associated with a medical personnel entity, a transaction request to perform an operation on the hyperledger, wherein the transaction request comprises search criteria, and the operation comprises providing, based on the search criteria, content pertaining to healthcare that is stored in the hyperledger;
executing one or more rules of the hyperledger to determine whether to allow the operation to be performed, wherein at least one of the one or more rules comprises determining whether the medical personnel entity has a right to access the content; and
responsive to determining that the one or more rules of the hyperledger are satisfied, performing the operation on the hyperledger to provide, based on the search criteria, the content pertaining to healthcare to the computing device.

Clause 116. The method of claim 14, further comprising:
receiving, from the computing device, a second transaction request to perform a second operation on the hyperledger, wherein the second operation comprises storing second content pertaining to healthcare in the hyperledger;
executing the one or more rules of the hyperledger to determine whether to allow the second operation to be performed, wherein at least a second rule of the one or more rules comprises determining whether the medical personnel entity is associated with an authorizing credential pertaining to healthcare; and
responsive to determining that the one or more rules of the hyperledger are satisfied, performing the second operation on the hyperledger to store the second content in the hyperledger.

Clause 117. The method of claim 14, wherein the right comprises the medical personnel entity being included in an organization that has viewing privileges of the content, the medical personnel entity being associated with an authorizing credential pertaining to healthcare, or some combination thereof.

Clause 118. The method of claim 14, further comprising:
receiving, from the computing device, a second transaction request to perform a second operation on the hyperledger, wherein the second operation comprises storing updated content pertaining to healthcare in the hyperledger, and the updated content adds additional content to original content included in the content stored in the hyperledger;
executing the one or more rules of the hyperledger to determine whether to allow the operation to be performed, wherein at least a second rule of the one or more rules comprises determining whether the additional content in the updated content is new relative to other content pertaining to healthcare stored in the hyperledger; and
responsive to determining that the one or more rules of the hyperledger are satisfied, performing the second operation on the hyperledger to store, in the hyperledger, the updated content including the additional content and the original content.

Clause 119. A method for maintaining content pertaining to healthcare in a hyperledger, the method comprising:
receiving, from a computing device associated with a medical personnel entity, a transaction request to perform an operation on the hyperledger, wherein the operation comprises storing updated content pertaining to healthcare in the hyperledger, and the updated content adds additional content to original content stored in the hyperledger;
executing one or more rules of the hyperledger to determine whether to allow the operation to be performed, wherein at least one of the one or more rules comprises determining whether the additional content in the updated content is new relative to other content pertaining to healthcare stored in the hyperledger; and
responsive to determining that the one or more rules of the hyperledger are satisfied, performing the operation on the hyperledger to store, in the hyperledger, the updated content including the additional content and the original content.

Clause 120. The method of claim 18, further comprising:
receiving, from the computing device, a second transaction request to perform a second operation on the hyperledger, wherein the second operation comprises storing second content pertaining to healthcare in the hyperledger;
executing the one or more rules of the hyperledger to determine whether to allow the operation to be performed, wherein at least a second rule of the one or more rules comprises determining whether the medical personnel entity is associated with an authorizing credential pertaining to healthcare; and
responsive to determining that the one or more rules of the hyperledger are satisfied, performing the second operation on the hyperledger to store the second content in the hyperledger.

Clause 121. The method of claim 18, further comprising:
receiving, from the computing device, a second transaction request to perform a second operation on the hyperledger, wherein the second transaction request comprises search criteria, and the second operation comprises providing, based on the search criteria, the updated content pertaining to healthcare that is stored in the hyperledger;
executing the one or more rules of the hyperledger to determine whether to allow the second operation to be performed, wherein at least a second rule of the one or more rules comprises determining whether the medical personnel entity has a right to access the updated content; and
responsive to determining that the one or more rules of the hyperledger are satisfied, performing the second operation on the hyperledger to provide, based on the search criteria, the updated content pertaining to healthcare to the computing device.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

What is claimed is:

1. A method for maintaining content pertaining to healthcare in a hyperledger, the method comprising:
receiving, from a computing device associated with a medical personnel entity, a transaction request to perform an operation on the hyperledger, wherein the operation comprises storing content pertaining to healthcare in the hyperledger;
executing one or more rules of the hyperledger to determine whether to allow the operation to be performed, wherein at least one of the one or more rules comprises determining whether the medical personnel entity is associated with an authorizing credential pertaining to healthcare; and
responsive to determining that the one or more rules of the hyperledger are satisfied, performing the operation on the hyperledger to store the content in the hyperledger.

2. The method of claim 1, wherein the authorizing credential is stored at a transaction in the hyperledger or the authorizing credential is included in the transaction request.

3. The method of claim 1, wherein the one or more rules comprise determining, using one or more transactions stored in the hyperledger, whether at least a portion of the content is new relative to other content in the hyperledger.

4. The method of claim 1, wherein the authorizing credential comprises a national provider identifier, and the one or more rules comprise determining, using one or more transactions stored in the hyperledger, whether the authorizing credential is valid.

5. The method of claim 1, further comprising validating authenticating credentials, authorizing credentials, or both prior to allowing the content to be stored in the hyperledger, wherein the authenticating credentials are provided to the computing device during registration and the authorizing credentials pertain to a medical license.

6. The method of claim 1, further comprising, responsive to determining that the one or more rules of the hyperledger are not satisfied, preventing the operation from being performed on the hyperledger.

7. The method of claim 1, wherein the content comprises a treatment plan, wherein at least a portion of the treatment plan is written by the medical personnel entity.

8. The method of claim 1, further comprising:
receiving, from the computing device, a second transaction request to perform a second operation on the hyperledger, wherein the second transaction request comprises search criteria, and the second operation comprises providing, based on the search criteria, a treatment plan pertaining to healthcare that is stored in the hyperledger;
executing the one or more rules of the hyperledger to determine whether to allow the operation to be performed, wherein at least a second rule of the one or more rules comprises determining whether the medical personnel entity has a right to access the content; and
responsive to determining that the one or more rules of the hyperledger are satisfied, performing the second operation on the hyperledger to provide, based on the search criteria, the treatment plan pertaining to healthcare to the computing device.

9. The method of claim 1, further comprising:
receiving, from the computing device associated, a second transaction request to perform a second operation on the hyperledger, wherein the second operation comprises storing updated content pertaining to healthcare in the hyperledger, and the updated document adds additional content to original content included in the content stored in the hyperledger;
executing the one or more rules of the hyperledger to determine whether to allow the operation to be performed, wherein at least a second rule of the one or more rules comprises determining whether the additional content in the updated content is new relative to other content pertaining to healthcare stored in the hyperledger; and
responsive to determining that the one or more rules of the hyperledger are satisfied, performing the second operation on the hyperledger to store, in the hyperledger, the updated content including the additional content and the original content.

10. The method of claim 1, further comprising maintaining a time series for the content that indicates when the content was stored, modified, validated, or some combination thereof.

11. The method of claim 1, further comprising:
receiving, from a computing device associated with a second medical personnel entity, a second transaction request to perform a second operation on the hyperledger, wherein the second operation comprises verifying the content in the hyperledger;
determining whether the second medical personnel entity is associated with a valid authorizing credential; and
responsive to determining that the second medical personnel entity is associated with the valid authoring credential, performing the second operation on the hyperledger by allowing the second medical personnel entity to verify the content.

12. The method of claim 1, further comprising:
receiving, from a second computing device, a transaction request to perform a second operation on the hyperledger, wherein the second operation comprises determining whether the content is trustworthy;
determining, using the hyperledger, a source of the content;
determining that the source is associated with the authorizing credential;
determining whether the content has been verified within a certain time period; and
providing a notification to the second computing device that the content is trustworthy based on the source of the content being associated with the authorizing credential and the content being verified within the certain time period.

13. The method of claim 1, wherein the content comprises a treatment plan for a medical issue, the medical personnel entity is a licensed medical doctor, and the hyperledger maintains different treatment plans for different licensed medical doctors that are validated as being provided by licensed medical doctors based on authorizing credentials that are stored in the hyperledger.

14. A method for maintaining content pertaining to healthcare in a hyperledger, the method comprising:
receiving, from a computing device associated with a medical personnel entity, a transaction request to perform an operation on the hyperledger, wherein the transaction request comprises search criteria, and the operation comprises providing, based on the search criteria, content pertaining to healthcare that is stored in the hyperledger;
executing one or more rules of the hyperledger to determine whether to allow the operation to be performed, wherein at least one of the one or more rules comprises determining whether the medical personnel entity has a right to access the content; and
responsive to determining that the one or more rules of the hyperledger are satisfied, performing the operation on the hyperledger to provide, based on the search criteria, the content pertaining to healthcare to the computing device.

15. The method of claim 14, further comprising:
receiving, from the computing device, a second transaction request to perform a second operation on the hyperledger, wherein the second operation comprises storing second content pertaining to healthcare in the hyperledger;
executing the one or more rules of the hyperledger to determine whether to allow the second operation to be performed, wherein at least a second rule of the one or more rules comprises determining whether the medical personnel entity is associated with an authorizing credential pertaining to healthcare; and
responsive to determining that the one or more rules of the hyperledger are satisfied, performing the second operation on the hyperledger to store the second content in the hyperledger.

16. The method of claim 14, wherein the right comprises the medical personnel entity being included in an organization that has viewing privileges of the content, the medical personnel entity being associated with an authorizing credential pertaining to healthcare, or some combination thereof.

17. The method of claim 14, further comprising:
receiving, from the computing device, a second transaction request to perform a second operation on the hyperledger, wherein the second operation comprises storing updated content pertaining to healthcare in the hyperledger, and the updated content adds additional content to original content included in the content stored in the hyperledger;
executing the one or more rules of the hyperledger to determine whether to allow the operation to be performed, wherein at least a second rule of the one or more rules comprises determining whether the additional content in the updated content is new relative to other content pertaining to healthcare stored in the hyperledger; and
responsive to determining that the one or more rules of the hyperledger are satisfied, performing the second operation on the hyperledger to store, in the hyperledger, the updated content including the additional content and the original content.

18. A method for maintaining content pertaining to healthcare in a hyperledger, the method comprising:
receiving, from a computing device associated with a medical personnel entity, a transaction request to perform an operation on the hyperledger, wherein the operation comprises storing updated content pertaining to healthcare in the hyperledger, and the updated content adds additional content to original content stored in the hyperledger;
executing one or more rules of the hyperledger to determine whether to allow the operation to be performed, wherein at least one of the one or more rules comprises determining whether the additional content in the updated content is new relative to other content pertaining to healthcare stored in the hyperledger; and
responsive to determining that the one or more rules of the hyperledger are satisfied, performing the operation on the hyperledger to store, in the hyperledger, the updated content including the additional content and the original content.

19. The method of claim 18, further comprising:
receiving, from the computing device, a second transaction request to perform a second operation on the hyperledger, wherein the second operation comprises storing second content pertaining to healthcare in the hyperledger;
executing the one or more rules of the hyperledger to determine whether to allow the operation to be performed, wherein at least a second rule of the one or more rules comprises determining whether the medical personnel entity is associated with an authorizing credential pertaining to healthcare; and
responsive to determining that the one or more rules of the hyperledger are satisfied, performing the second operation on the hyperledger to store the second content in the hyperledger.

20. The method of claim 18, further comprising:
receiving, from the computing device, a second transaction request to perform a second operation on the hyperledger, wherein the second transaction request comprises search criteria, and the second operation comprises providing, based on the search criteria, the updated content pertaining to healthcare that is stored in the hyperledger;
executing the one or more rules of the hyperledger to determine whether to allow the second operation to be performed, wherein at least a second rule of the one or more rules comprises determining whether the medical personnel entity has a right to access the updated content; and
responsive to determining that the one or more rules of the hyperledger are satisfied, performing the second operation on the hyperledger to provide, based on the search criteria, the updated content pertaining to healthcare to the computing device.
